(19) United States
(12) Patent Application Publication
(10) Pub. No.: US 2011/0144345 A1
(43) Pub. Date: Jun. 16, 2011

(54) SULFOXYIMINO-SUBSTITUTED BENZOYL DERIVATIVE AND HERBICIDE

(75) Inventors: Tetsuo Tamai, Naka-gun (JP); Kazuaki Tsukuda, Sakura-shi (JP); Shigeo Yamada, Odawara-shi (JP); Yasuhiro Miyashita, Odawara-shi (JP); Shingo Hirai, Naka-gun (JP)

(73) Assignee: NIPPON SODA CO., LTD., Tokyo (JP)

(21) Appl. No.: 13/057,624

(22) PCT Filed: Aug. 3, 2009
(86) PCT No.: PCT/JP2009/003696

§ 371 Date: Feb. 4, 2011

(30) Foreign Application Priority Data
Aug. 5, 2008 (JP) 2008-202445

(51) Int. Cl. C07D 409/14; C07D 335/02; C07D 409/12; C07D 327/06; C07D 411/12; C07C 313/06; C07D 231/20; C07C 255/41

(52) U.S. Cl. 546/211; 549/28; 548/365.7; 549/14; 564/102; 548/369.4; 558/405

(57) ABSTRACT

The benzoyl derivative of the present invention is represented by formula (I)

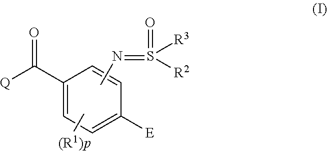

(in the formula, E represents an alkoxy group, an alkoxycarbonyl group or the like, R¹ represents a halogen atom, an organic group or the like, p represents an integer of 0 to 3, R² and R³ each independently represents an alkyl group or the like, Q represents a group selected from the groups represented by the following formulas Q1 to Q8:

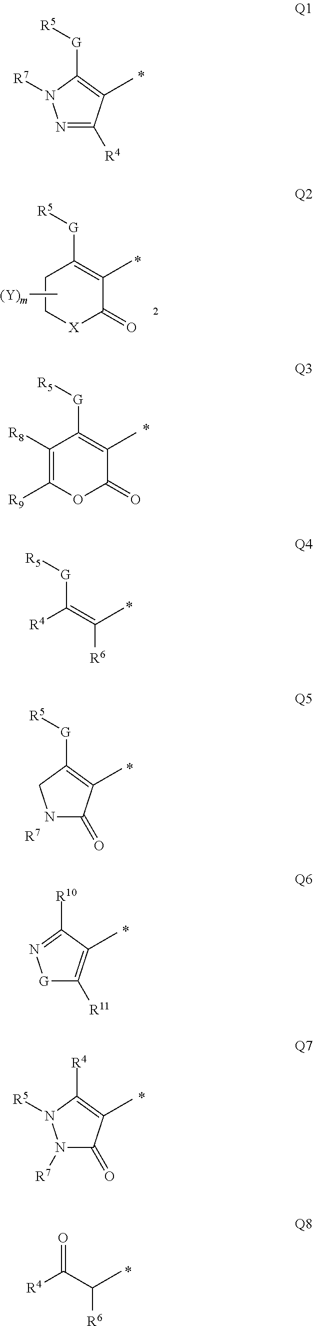

(in the formula, * represents binding site, G represents oxygen atom or the like), R⁴ to R⁵, R⁸ to R¹³ represents hydrogen atom, an alkyl group or the like), R⁶ represents cyano group or the like, X represents —C(R¹²)(R¹³)— or —N(R¹²)—, Y represents oxo group, an alkyl group or the like, m represents an integer of 0 to 4)) or salt thereof.

### TECHNICAL FIELD

The present invention relates to a benzoyl derivative or salt thereof having sulfoxyimino group, and a herbicide including one or two or more types of the compounds as active ingredient.

Priority is claimed on Japanese Patent Application No. 2008-202445, filed Aug. 5, 2008, the content of which is incorporated herein by reference.

### BACKGROUND ART

Many herbicides are being used for weed control, which has required intensive labor in the past when growing field and garden crops. However, the development of drugs which are reliably effective at a lesser dose and which are also possible to use safely is desired due to the occurrence of chemical damage to crops, environmental persistence of the drugs, and environmental pollution caused by the drugs.

Several patent documents disclose that benzoic acid derivatives which are similar to the compound of the present invention have herbicidal activity.

For example, Patent document 1 discloses that a benzoic acid derivative represented by the following formula is effective as an active ingredient of herbicide. In addition, Patent document 1 also discloses that the compounds described in this patent document are useful for an active ingredient of herbicide.

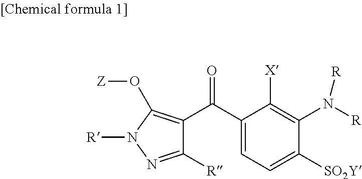

(In the formula, Y′ represents methyl group or the like, Z represents hydrogen atom or the like, X′ represents a halogen atom or the like, R, R′ and R″ each independently represents an alkyl group or the like.)

However, the compound of the present invention is not described in this patent document.

In addition, Patent document 2 discloses that a benzoic acid derivative represented by the following formula is effective as an active ingredient of herbicide.

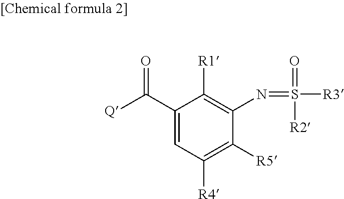

(In the formula, R1′ to R5′ each independently represents hydrogen atom or the like, Q′ represents the groups represented by the following formulas (Q′-1) to (Q′-3).

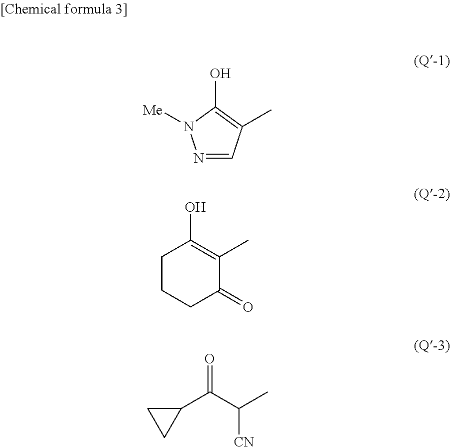

Although the compound represented by the general formula described in this patent document includes the compound of the present invention, there is only the compound having methylsulfonyl group (SO₂Me) at 4-position of benzoyl group is practically synthesized in this patent document, and this patent document does not describe a specific example of the compound of the present invention.

### PRIOR ART LITERATURE

### Patent Literature

Patent document 1: publication of WO98/42678
Patent document 2: publication of WO04/52849

### DISCLOSURE OF INVENTION

### Problems to be Solved by the Invention

The objective of the present invention is to provide a compound for herbicide which is reliably effective when used in a low dose and is highly safe.

### Means for Solving the Problems

As a result of intensive research, the present inventors discovered that the compound represented by formula (I) is particularly useful for an active ingredient of herbicide, and completed the present invention.

Namely, the present invention relates to the following.

(1) A benzoyl derivative represented by formula (I)

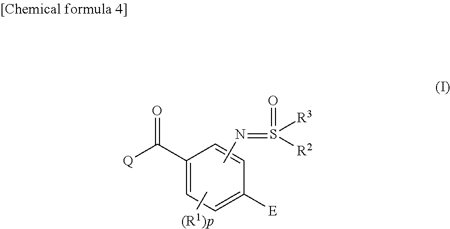

(In the formula, E represents hydrogen atom, an alkyl group, an alkenyl group, an alkynyl group, an aryl group, an alkoxy group, a cycloalkoxy group, an alkoxycarbonyl group, an alkyl thio group, cyano group, an acyl group, a heterocyclic group, NR^{a}₂ group (in the formula, each R^{a} independently represents hydrogen atom or a hydrocarbon group), R^{a}₂NC(O) group (in the formula, R^{a} is as defined above), NR^{c}C(O)R^{a} (in the formula, R^{a} is as defined above, R^{c} represents hydrogen atom or an alkyl group), NR^{c}CO₂R^{a} (in the formula, R^{a} and R^{c} are as defined above), or CR^{c}═NOR^{d} (in the formula, R^{c} is as defined above, R^{d} represents hydrogen atom or an alkyl group), when E represents NR^{a}₂ group or R^{a}₂NC(O) group, two R^{a} may bond together to form a 3- to 6-membered ring,

R¹ represents a halogen atom, hydroxyl group, mercapto group, NR^{a}₂ group (in the formula, R^{a} is as defined above), nitro group or an organic group,

p represents an integer of 0 to 3, when p is 2 or more, the numerous R¹ may be the same or different from each other,

R² and R³ each independently represents an alkyl group, a cycloalkyl group, an alkenyl group, an alkynyl group, an aryl group or a heterocyclic group,

and R² and R³ may bond together to form a 3- to 8-membered hetero ring which may have 1 to 4 nitrogen atoms, oxygen atoms or sulfur atoms other than the sulfur atom in sulfoxyimino group,

Q represents a group selected from the groups represented by the following formulas Q1 to Q8:

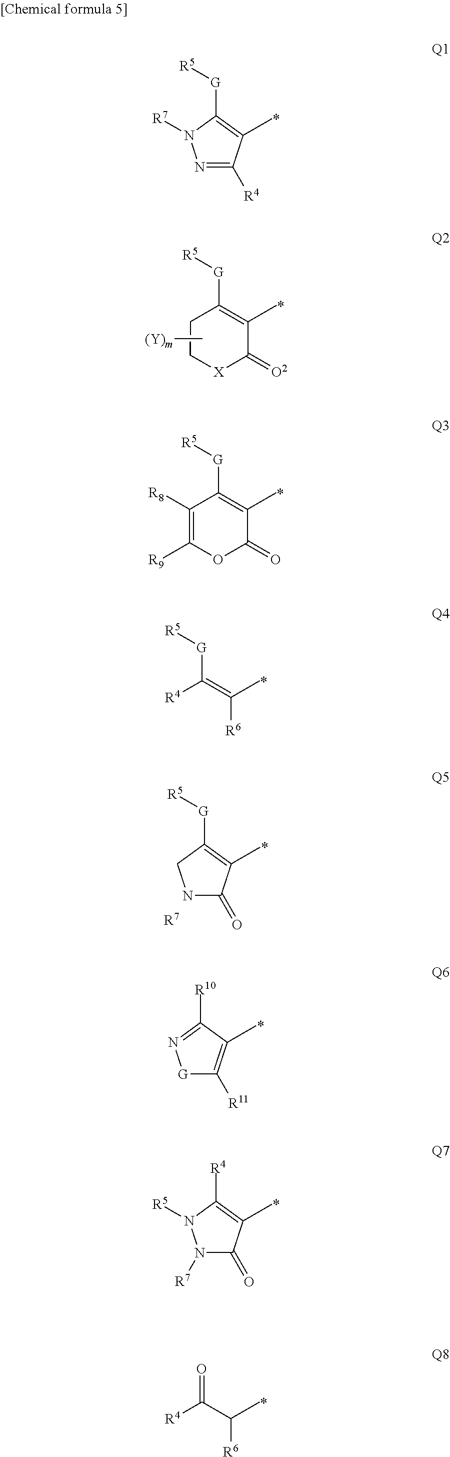

(In the formula, * represents binding site,

G represents oxygen atom, —S—, —S(O)—, —S(O)₂— or —NR^{b}— (in the formula, R^{b} represents hydrogen atom or an organic group),

R⁴ represents hydrogen atom, an alkyl group, a cycloalkyl group or NR^{a}₂ group (in the formula, R^{a} is as defined above),

R⁵ represents hydrogen atom, an alkyl group, an alkenyl group, an alknyl group, an aryl group, a heteroaryl group, an alkoxycarbonyl group, an alkyl thiocarbonyl group, an acyl group, R^{a}₂NC(O) group (in the formula, R^{a} is as defined above), an alkylsulfonyl group, an arylsulfonyl group or NR^{a}₂SO₂ group (in the formula, R^{a} is as defined above),

R⁶ represents cyano group, an acyl group, an alkoxycarbonyl group, —C(R⁷¹)═NR⁷ group (in the formula, R⁷¹ represents hydrogen atom, an alkyl group, an aryl group or a heteroaryl group, R⁷ represents hydrogen atom, an alkyl group or an alkoxy group) or a tetrazolyl group,

R⁸ and R⁹ each independently represents hydrogen group or an alkyl group,

R¹⁰ and R¹¹ each independently represents hydrogen atom, an alkyl group or a cycloalkyl group,

X represents —C(R¹²)(R¹³)— or —N(R¹²)— (in the formula, R¹² and R¹³ each independently represents hydrogen atom or an alkyl group),

Y represents oxo group, an alkyl group, an alkoxy group, an acyl group or an alkoxycarbonyl group,

m represents an integer of 0 to 4,

when m is 2 or more, the numerous Y may be the same or different from each other, Y may bond with each other to form a ring regardless of the substitutents listed above, Y and R¹² of X may bond together to form a ring regardless of the substitutents listed above)) or salt thereof.

(2) The benzoyl derivative or salt thereof according to (1), wherein the benzoyl derivative is represented by formula (1-b):

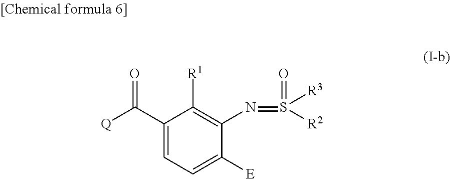

(In the formula, E, R¹ to R³, and Q are as defined above).

(3) The benzoyl derivative or salt thereof according to (1) or (2), wherein in the formulas, R¹ represents a halogen atom, an alkyl group or —N═S(═O)R²R³ (in the formula, R² and R³ are as defined above).

(4) The benzoyl derivative or salt thereof according to any one of (1) to (3), wherein in the formulas, E represents an alkoxy group or an alkoxycarbonyl group.

(5) A herbicide comprising at least one type of benzoyl derivative or salt thereof according to any one of (1) to (4) as an active ingredient.

### Effects of the Invention

The composition of the present invention, which includes as an active ingredient one or two or more types of benzoyl derivatives having sulfoxyimino group or salts thereof is useful for herbicide which is reliably effective when used in a low dose and is highly safe.

### BEST MODE FOR CARRYING OUT THE INVENTION

Hereinafter, the present invention will be described in detail.

### 1) Benzoyl Derivative or Salt Thereof Having Sulfoxyimino Group

The first aspect of the present invention is a benzoyl derivative having sulfoxyimino group represented by formula (I):

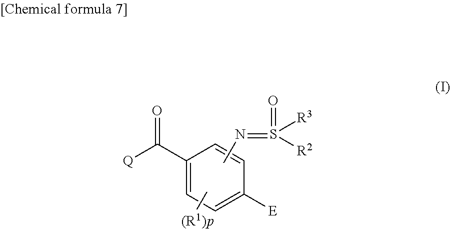

(In the formula, E represents hydrogen atom, an alkyl group, an alkenyl group, an alkynyl group, an aryl group, an alkoxy group, a cycloalkoxy group, an alkoxycarbonyl group, an alkyl thio group, cyano group, an acyl group, a heterocyclic group, NR^{a}₂ group (in the formula, each R^{a} independently represents hydrogen atom or a hydrocarbon group), R^{a}₂NC(O) group (in the formula, R^{a} is as defined above), NR^{c}C(O)R^{a} (in the formula, R^{a} is as defined above, R^{c} represents hydrogen atom or an alkyl group), NR^{c}CO₂R^{a} (in the formula, R^{a} and R^{c} are as defined above), or CR^{c}═NOR^{d} (in the formula, R^{c} is as defined above, R^{d} represents hydrogen atom or an alkyl group), when E represents NR^{a}₂ group or R^{a}₂NC(O) group, two R^{a} may bond together to form a 3- to 6-membered ring,

R¹ represents a halogen atom, hydroxyl group, mercapto group, NR^{a}₂ group (in the formula, R^{a} is as defined above), nitro group or an organic group,

p represents an integer of 0 to 3, when p is 2 or more, the numerous R¹ may be the same or different from each other,

R² and R³ each independently represents an alkyl group, a cycloalkyl group, an alkenyl group, an alkynyl group, an aryl group or a heterocyclic group,

R² and R³ may bond together to form a 3- to 8-membered hetero ring which may have 1 to 4 nitrogen atoms, oxygen atoms or sulfur atoms other than the sulfur atom in sulfoxyimino group,

Q represents a group selected from the groups represented by the following formulas Q1 to Q8:

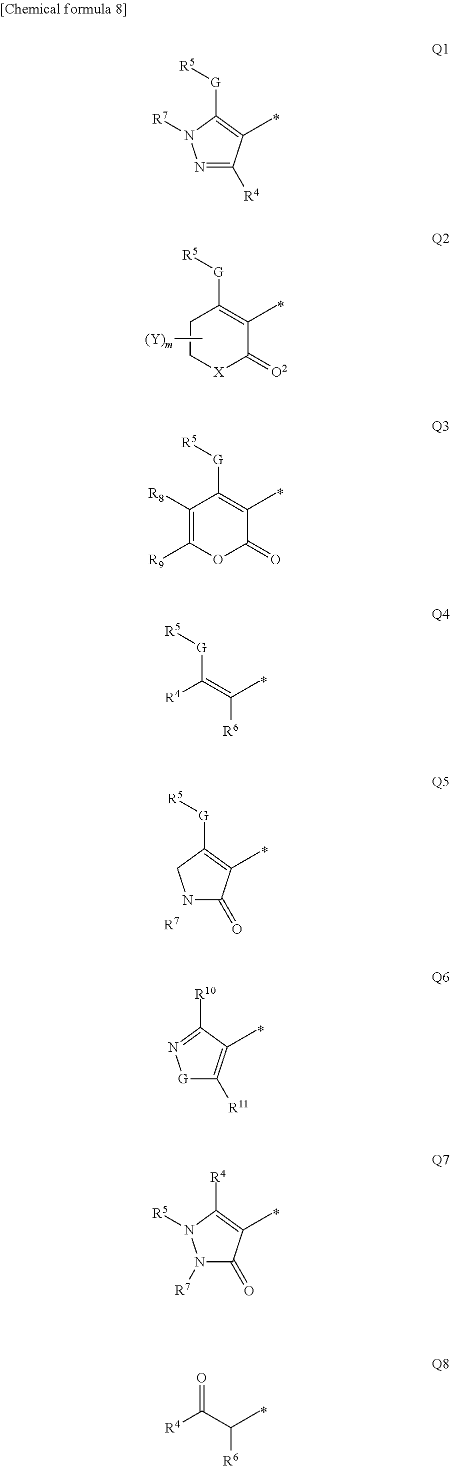

(In the formula, * represents binding site,

G represents oxygen atom, —S—, —S(O)—, —S(O)₂— or —NR^{b}— (in the formula, R^{b} represents hydrogen atom or an organic group),

R⁴ represents hydrogen atom, an alkyl group, a cycloalkyl group or NR^{a}₂ group (in the formula, R^{a} is as defined above),

R⁵ represents hydrogen atom, an alkyl group, an alkenyl group, an alknyl group, an aryl group, a heteroaryl group, an alkoxycarbonyl group, an alkyl thiocarbonyl group, an acyl group, R^{a}₂NC(O) group (in the formula, R^{a} is as defined above), an alkylsulfonyl group, an arylsulfonyl group or NR^{a}₂SO₂ group (in the formula, R^{a} is as defined above),

R⁶ represents cyano group, an acyl group, an alkoxycarbonyl group, —C(R⁷¹)═NR⁷ group (in the formula, Rⁿ represents hydrogen atom, an alkyl group, an aryl group or a heteroaryl group, R⁷ represents hydrogen atom, an alkyl group or an alkoxy group) or a tetrazolyl group,

R⁸ and R⁹ each independently represents hydrogen group or an alkyl group,

R¹⁰ and R¹¹ each independently represents hydrogen atom, an alkyl group or a cycloalkyl group,

X represents —C(R¹²)(R¹³)— or —N(R¹²)— (in the formula, R¹² and R¹³ each independently represents hydrogen atom or an alkyl group),

Y represents oxo group, an alkyl group, an alkoxy group, an acyl group or an alkoxycarbonyl group,

m represents an integer of 0 to 4,

when m is 2 or more, the numerous Y may be the same or different from each other, Y may bond with each other to form a ring regardless of the substitutents listed above, Y and R¹² of X may bond together to form a ring regardless of the substitutents listed above)) (hereinafter referred to as “the compound of the present invention”) or salt thereof. The compound of the present invention or salt thereof includes hydrate, various solvates, crystalline polymorphism and the like.

The groups like will be described in detail below. Each group may include one or more types, or one or more substituents within a chemically acceptable range. In addition; the number of carbon atoms described below does not include the number of carbon atoms in the substituents when the group is substituented.

### (E)

In the present invention, E represents hydrogen atom, an alkyl group, an alkenyl group, an aryl group, an alkoxy group, a cycloalkoxy group, an alkoxycarbonyl group, an alkyl thio group, cyano group, an acyl group, a heterocyclic group, NR^{a}₂ group (in the formula, each R^{a} independently represents hydrogen atom or a hydrocarbon group), R^{a}₂NC(O) group (in the formula, R^{a} is as defined above), NR^{c}C(O)R^{a} (in the formula, R^{a} is as defined above, R^{c} represents hydrogen atom or an alkyl group), NR^{c}CO₂R^{a} (in the formula, R^{a} and R^{c} are as defined above), or CR^{c}═NOR^{d} (in the formula, R^{c} is as defined above, R^{d} represents hydrogen atom or an alkyl group), when E represents NR^{a}₂ group or R^{a}₂NC(O) group, two R^{a} may bond together to form a 3- to 6-membered ring,

“Alkyl group” indicates a linear or branched alkyl group. Examples of “alkyl group” include methyl group, ethyl group, n-propyl group, i-propyl group, n-butyl group, i-butyl group, s-butyl group, t-butyl group, n-pentyl group, n-hexyl group and the like. Among these examples, a C1-6 alkyl group is preferable.

Within a chemically acceptable range, “alkyl group” may have one or more types of, or one or more substituents selected from the substituents described in TABLE 1. The substituents are preferably hydroxy group, thiole group, cyano group, isocyano group, nitro group, isocyanato group, isothiocyanato group, cyanato group, thiocyanato group, carboxyl group, amino group, a cycloalkyl group, a cycloalkenyl group, an aryl group, an unsaturated 5-membered heterocyclic group, an unsaturated 6-membered heterocyclic group, a saturated heterocyclic group, a monoalkyl amino group, a monoaryl amino group, a dialkyl amino group, a diaryl amino group, an alkyl sulfonyl amino group, an aryl sulfonyl amino group, a heteroaryl sulfonyl amino group, an alkyl carbonyl amino group, an alkoxycarbonyl amino group, a bis(alkyl sulfonyl)amino group, an alkoxy group, a haloalkoxy group, an alkenyloxy group, an alkynyloxy group, an aryloxy group, an alkoxycarbonyloxy group, an aryl alkyloxy group, a heteroring oxy group, an alkyl thiocarbonyl group, an alkoxycarbonyl group, a substituted or unsubstituted aminocarbonyl group, a substituted or unsubstituted hydrazino group, a substituted or unsubstituted hydrazinocarbonyl group, an alkyl thio group, an alkenyl thio group, an alkynyl thio group, an aryl thio group, a heteroaryl thio group, an aryl alkyl thio group, an alkyl sulfonyl group, an alkenyl sulfonyl group, an alkynyl sulfonyl group, an aryl sulfonyl group, a heteroaryl sulfonyl group, an aryl alkyl sulfonyl group, an acyl group, and an acyloxy group, and particularly preferably an alkoxy group.

Examples of “alkenyl group” include ethenyl group, 1-propenyl group, 2-propenyl group, 1-butenyl group, 2-butenyl group, 3-butenyl group, 1-methyl-2-propenyl group, 2-methyl-2-propenyl group, 1-pentenyl group, 2-pentenyl group, 3-pentenyl group, 4-pentenyl group, 1-methyl-2-butenyl group, 2-methyl-2-butenyl group, 1-hexenyl group, 2-hexenyl group, 3-hexenyl group, 4-hexenyl group and 5-hexenyl group and the like. Among these examples, an alkenyl group having 2 to 6 carbon atoms is preferable. Within a chemically acceptable range, “alkenyl group” may have one or more types of substituents, or one or more substituents selected from the substituents described in TABLE 1.

Examples of “alkynyl group” include ethynyl group, 1-propynyl group, 2-propynyl group, 1-butynyl group, 2-butynyl group, 3-butyryl group, 1-methyl-2-propynyl group, 2-methyl-3-butynyl group, 1-pentynyl group, 2-pentynyl group, 3-pentynyl group, 4-pentynyl group, 1-methyl-2-butynyl group, 2-methyl-3-pentynyl group, 1-hexynyl group, 1,1-dimethyl-2-butynyl group and the like. Among these examples an alkynyl group having 2 to 6 carbon atoms is preferable. Within a chemically acceptable range, “alkynyl group” may have one or more types of substituents selected from the substituents described in TABLE 1.

“Aryl group” indicates a monocyclic or polycyclic aryl group. The polycyclic aryl group includes a fully-unsaturated group as well as a partially-unsaturated group. Examples of “aryl group” include phenyl group, 1-naphthyl group, 2-naphthyl group, azulenyl group, indenyl group, indanyl group, tetralinyl group and the like. Among these examples an aryl group having 6 to 14 carbon atoms is preferable. Within a chemically acceptable range, “aryl group” may have one or more types of substituents, or one or more substituents selected from the substituents described in TABLE 1.

Examples of “alkoxy group” include methoxy group, ethoxy group, n-propoxy group, i-propoxy group, n-butoxy group, i-butoxy group, s-butoxy group, t-butoxy group, n-pentyloxy group, n-hexyloxy group and the like. Among these examples an alkoxy group having 1 to 6 carbon atoms is preferable.

Within a chemically acceptable range, “alkoxy group” may have one or more types of substituents, or one or more substituents selected from the substituents described in TABLE 1. The substituents are preferably hydroxy group, thiole group, a halogen atom, cyano group, isocyano group, nitro group, isocyanato group, isothiocyanato group, cyanato group, thiocyanato group, carboxyl group, amino group, a cycloalkyl group, an alkenyl group, a cycloalkenyl group, an alkynyl group, a haloalkyl group, an aryl group, an unsaturated 5-membered heterocyclic group, an unsaturated 6-membered heterocyclic group, a saturated heterocyclic group, a monoalkyl amino group, a monoaryl amino group, a dialkyl amino group, a diaryl amino group, an alkyl sulfonyl amino group, an aryl sulfonyl amino group, a heteroaryl sulfonyl amino group, an alkyl carbonyl amino group, an alkoxycarbonyl amino group, a bis(alkyl sulfonyl)amino group, an alkoxy group, a haloalkoxy group, an alkenyloxy group, an alkynyloxy group, an aryloxy group, an alkoxycarbonyloxy group, an aryl alkyloxy group, a heteroring oxy group, an alkyl thiocarbonyl group, an alkoxycarbonyl group, a substituted or unsubstituted aminocarbonyl group, a substituted or unsubstituted hydrazino group, a substituted or unsubstituted hydrazinocarbonyl group, an alkyl thio group, an alkenyl thio group, an alkynyl thio group, an aryl thio group, a heteroaryl thio group, an aryl alkyl thio group, an alkyl sulfonyl group, an alkenyl sulfonyl group, an alkynyl sulfonyl group, an aryl sulfonyl group, a heteroaryl sulfonyl group, an aryl alkyl sulfonyl group, an acyl group, and an acyloxy group.

“Cycloalkoxy group” indicates a group in which oxygen atom bond to an alkyl group having a monocyclic moiety or a polycyclic moiety. Examples of “cycloalkoxy group” include cyclopropyloxy group, cyclopentyloxy group, cyclohexyloxy group, cyclopropyl methyloxy group, cyclopentyl methyloxy group and the like. “Cycloalkoxy group” is preferably a C3-C8 cycloalkoxy group. Within a chemically acceptable range, “cycloalkoxy group” may have one or more types of substituents, or one or more substituents selected from the substituents described in TABLE 1.

Examples of “alkoxycarbonyl group” include methoxycarbonyl group, ethoxycarbonyl group, n-propoxycarbonyl group, i-propoxycarbonyl group, n-butoxycarbonyl group, i-butoxycarbonyl group, s-butoxycarbonyl group, t-butoxycarbonyl group, n-pentyloxycarbonyl group, n-hexyloxycarbonyl group and the like. Among these examples, an alkoxycarbonyl group having 2 to 6 carbon atoms are preferable. Within a chemically acceptable range, “alkoxycarbonyl group” may have one or more types of substituents, or one or more substituents selected from the substituents described in TABLE 1.

Examples of “alkyl thio group” include methyl thio group, ethyl thio group, n-propyl thio group, i-propyl thio group, n-butyl thio group, i-butyl thio group, s-butyl thio group, t-butyl thio group and the like. Among these examples, an alkyl thio group having 1 to 6 carbon atoms is preferable.

Within a chemically acceptable range, “alkyl thio group” may have one or more types of substituents, or one or more substituents selected from the substituents described in TABLE 1. The substituents are preferably hydroxy group, thiole group, a halogen atom, cyano group, isocyano group, nitro group, isocyanato group, isothiocyanato group, cyanato group, thiocyanato group, carboxyl group, amino group, a cycloalkyl group, a cycloalkenyl group, an aryl group, an unsaturated 5-membered heterocyclic group, an unsaturated 6-membered heterocyclic group, a saturated heterocyclic group, a monoalkyl amino group, a monoaryl amino group, a dialkyl amino group, a diaryl amino group, an alkyl sulfonyl amino group, an aryl sulfonyl amino group, a heteroaryl sulfonyl amino group, an alkyl carbonyl amino group, an alkoxycarbonyl amino group, a bis(alkyl sulfonyl)amino group, an alkoxy group, a haloalkoxy group, an alkenyloxy group, a haloalkenyl group, an alkynyloxy group, an aryloxy group, an alkoxycarbonyloxy group, an aryl alkyloxy group, a heteroring oxy group, an alkyl thiocarbonyl group, an alkoxycarbonyl group, a substituted or unsubstituted aminocarbonyl group, a substituted or unsubstituted hydrazino group, a substituted or unsubstituted hydrazinocarbonyl group, an alkyl thio group, an alkenyl thio group, an alkynyl thio group, an aryl thio group, a heteroaryl thio group, an aryl alkyl thio group, an alkyl sulfonyl group, an alkenyl sulfonyl group, an alkynyl sulfonyl group, an aryl sulfonyl group, a heteroaryl sulfonyl group, an aryl alkyl sulfonyl group, an acyl group, an acyloxy group.

“Acyl group” indicates a group in which carbonyl group bond to hydrogen atom, an alkyl group, an alkenyl group, an alkynyl group, an aryl group, a hetero aryl group or the like. Examples of “acyl group” include formyl group;

an alkyl carbonyl group (preferably having 2 to 6 carbon atoms) such as acetyl group, propionyl group, butyroyl group, pentanoyl group, hexanoyl group, heptanoyl group, octanoyl group, nonanoyl group, decanoyl group, 3-methyl nonanoyl group, 8-methyl nonanoyl group, 3-ethyl octanoyl group, 3,7-dimethyl octanoyl group, undecanoyl group, dodecanoyl group, tridecanoyl group, tetradecanoyl group, pentadecanoyl group, hexadecanoyl group, 1-methyl pentadecanoyl group, 14-methyl pentadecanoyl group, 13,13-dimethyl tetradecanoyl group, heptadecanoyl group, 15-methyl hexadecanoyl group, octadecanoyl group, 1-methyl heptadecanoyl group, nonadecanoyl group, eicosanoyl group, heneicosanoyl group or the like;
an alkenyl carbonyl group (preferably having 3 to 6 carbon atoms) such as acryloyl group, allyl carbonyl group or the like;
an alkynyl carbonyl group (preferably having 3 to 6 carbon atoms) such as ethynyl carbonyl group, 2-propynyl carbonyl group or the like;
a cycloalkyl carbonyl group (preferably having 4 to 7 carbon atoms) such as cyclopropyl carbonyl group, cyclopentyl carbonyl group or the like;
an aryl carbonyl group (preferably having 7 to 15 carbon atoms) such as benzoyl group, napthyl carbonyl group, biphenyl carbonyl group, anthranicarbonyl group or the like; a hetero aryl carbonyl group (preferably having 7 to 15 carbon atoms) such as 2-pyridyl carbonyl group, 2-thienyl carbonyl group or the like. Within a chemically acceptable range, “acyl group” may have one or more types of substitutents, or one or more substituents selected from the substituents described in TABLE 1.

“Heterocyclic group” indicates a 5- to 7-membered heteroaromatic ring, a 5- to 7-membered saturated heterocyclic ring or a 5- to 7-membered unsaturated heterocyclic ring, which have 1 to 4 hetero atoms selected from nitrogen atom, oxygen atom and sulfur atom other than carbon atom, or indicates a condensed heterocyclic ring in which benzene ring and these heterocyclic rings are condensed. Examples of “heterocyclic group” include 2-furyl group, 3-furyl group, 2-thienyl group, 3-thienyl group, 2-oxazolyl group, 2-oxazolinyl group, 3-isoxazolyl group, 4-isoxazolyl group, 5-isoxazolyl group, 3-isoxazolinyl group, 2-thiazolyl group, 2-thiazolinyl group, 3-isothiazolyl group, 3-isothiazolinyl group, 2-pyranyl group, 4-tetrahydropyranyl group, 1-azetidinyl group, 2-azetidinyl group, 3-azetidinyl group, 2-pyrrolyl group, 2-pyrrolidinyl group, 2-imidazolyl group, 3-pyrazolyl group, 2-imidazolinyl group, 2-pyridyl group, 3-pyridyl group, 4-pyridyl group, 2-piperidyl group, piperidino group, 2-morpholinyl group, morpholino group, 2-piperazinyl group, 2-pyrimidinyl group, 3-pyridazinyl group, 2-pyrazinyl group and the like. Within a chemically acceptable range, “heterocyclic group” may have one or more types of substituents, or one or more substituents selected from the substituents described in TABLE 1.

R^{a} of “NR^{a}₂ group” represents hydrogen atom or a hydrocarbon group.

In the examples of R^{a}, “hydrocarbon group” represents an alkyl group, a cycloalkyl group, an alkenyl group, a cycloalkenyl group, an alkynyl group, an aryl group, an aryl alkyl group or the like.

In addition, two R^{a} may bond with each other to form a 3- to 6-membered ring.

Here, examples of “alkyl group”, “alkenyl group”, “alkynyl group”, and “aryl group” are as defined above.

“Cycloalkyl group” indicates an alkyl group having a monocyclic or a polycyclic moiety. Examples of “cycloalkyl group” include cyclopropyl group, cyclobutyl group, cyclopentyl group, cyclohexyl group, cyclopropyl methyl group, cyclohexyl methyl group and the like. Among these examples, a cycloalkyl group having 3 to 8 carbon atoms is preferable.

“Cycloalkenyl group” indicates an alkenyl group having a cyclic moiety. Examples of “cycloalkenyl group” include cyclopropenyl group, 2-cyclobutenyl group, 3-cyclopentenyl group, 4-cyclohexenyl group, 3-cyclopentenyl methyl group, 4-cyclohexenyl methyl group and the like. Among these examples, a cycloalkenyl group having 3 to 8 carbon atoms is preferable.

Examples of “arylalkyl group” include benzyl group, phenethyl group, 3-phenyl-n-propyl group, 1-phenyl-n-hexyl group, naphthalene-1-yl methyl group, naphthalene-2-yl ethyl group, 1-naphthalene-2-yl-n-propyl group, indene-1-yl methyl group and the like. Among these examples, a (C6 to 10) aryl (C1 to 6) alkyl group is preferable.

Examples of “Nr^{a}₂ group” include amino group, dimethyl amino group, methyl ethyl amino group, vinyl amino group, allyl amino group, phenyl amino group, benzyl amino group, pyrrolidine-2-yl group and the like.

Within a chemically acceptable range, “NR^{a}₂ group” may have one or more types of substituents, or one or more substituents selected from the substituents described in TABLE 1.

R^{a}₂NC(O) group is a group in which the above described NR^{a}₂ group and carbonyl group are bonded. Two R^{a} may bond with each other to form a 3- to 6-membered ring.

Examples of “R^{a}₂NC(O) group” include aminocarbonyl group, dimethyl aminocarbonyl group, methyl ethyl aminocarbonyl group, vinyl aminocarbonyl group, allyl aminocarbonyl group, phenyl aminocarbonyl group, benzyl aminocarbonyl group and the like.

Within a chemically acceptable range, “R^{a}₂NC(O) group” may have one or more types of substituents, or one or more substituents selected from the substituents described in TABLE 1.

R^{a} of “NR^{c}C(O)R^{a} group” is as defined above and R^{c} of “NR^{c}C(O)R^{a} group” represents hydrogen group or an alkyl group. Examples of an alkyl group of R^{c} are the same as the examples of an alkyl group of the above described E.

Examples of “NR^{c}C(O)R^{a} group” include acetyl amino group, propionyl amino group, benzoyl amino group, N-methyl acetyl amino group, N-i-propyl cyclohexyl carbonyl amino group and the like.

Within a chemically acceptable range, “NR^{c}C(O)R^{a} group” may have one or more types of substituents, or one or more substituents selected from the substituents described in TABLE 1.

R^{a} and R^{c} of “NR^{c}CO₂R^{a} group” are as defined above.

Examples of “NR^{c}CO₂R^{a} group” include methoxycarbonyl amino group, phenoxycarbonyl amino group, N-methyl-methoxycarbonyl amino group and the like.

Within a chemically acceptable range, “NR^{c}CO₂R^{a} group” may have one or more types of substituents, or one or more substituents selected from the substituents described in TABLE 1.

R^{c} of “CR^{c}═NOR^{d} group” is as defined above, and R^{d} of “CR^{c}═NOR^{d} group” represents hydrogen atom or an alkyl group. Examples of an alkyl group of R^{d} are the same as the examples of an alkyl group of the above described E.

Examples of “CR^{c}═NOR^{d} group” include CH═NOH, CH═NOMe, CMe=NOEt and the like.

Within a chemically acceptable range, CR^{c}═NOR^{d} group may have one or more types of substituents, or one or more substituents selected from the substituents described in TABLE 1.

E is preferably an alkoxy group or an alkoxycarbonyl group.

TABLE 1

| Type of substituent | Example |
|---|---|
| Hydroxyl group | |
| Thiol group | |
| Halogen atom | Fluorine atom, chlorine atom, bromine atom, iodine |
| | atom |
| Cyano group | |
| Isocyano group | |
| Nitro group | |
| Isocyanato group | |
| Isothiocyanato group | |
| Cyanato group | |
| Thiocyanato group | |
| Carboxyl group | |
| Amino group | |
| Alkyl group | Methyl group, ethyl group, n-propyl group, i-propyl |
| | group, n-butyl group, s-butyl group, i-butyl group, |
| | t-butyl group, n-pentyl group, n-hexyl group, n-decyl |
| | group, n-dodecyl group or the like, |
| | preferably C1-6 alkyl group |
| Cycloalkyl group | Cyclopropyl group, cyclobutyl group, cyclopentyl |
| | group, cyclohexyl group, cyclobutyl group or the like |
| | preferably C3-8 cycloalkyl group |
| Alkenyl group | Vinyl group, 1-propenyl group, 2-propenyl group, |
| | 1-butenyl group, 2-butenyl group, 3-butenyl group, |
| | 1-methyl-2-propenyl group, 2-methyl-2-propenyl |
| | group, 1-pentenyl group, 2-pentenyl group, 3-pentenyl |
| | group, 4-pentenyl group, 1-methyl-2-butenyl group, |
| | 2-methyl-2-butenyl group, 1-hexenyl group, |
| | 2-hexenyl group, 3-hexenyl group, 4-hexenyl group, |
| | 5-hexenyl group, 1-decenyl group or the like, |
| | preferably C2-6 alkenyl group |
| Cycloalkenyl group | Cyclopropenyl group, 2-cyclopentenyl group, |
| | 3-cyclohexenyl group, 4-cyclooctenyl group or the like, |
| | preferably C3-8 cycloalkenyl group |
| Alkynyl group | Ethynyl group, 1-propynyl group, 2-propynyl group, |
| | 1-butynyl group, 2-butynyl group, 3-butynyl group, |
| | 1-methyl-2-propynyl group, 2-methyl-3-butynyl group, |
| | 1-pentynyl group, 2-pentynyl group, 3-pentynyl group, |
| | 4-pentynyl group, 1-methyl-2-butynyl group, 2-methyl-3 |
| | pentynyl group, 1-hexynyl group, |
| | 1,1-dimethyl-2-butynyl group, 1-decynyl group or the |
| | like, |
| | preferably C2-6 alkynyl group |
| Haloalkyl group | Chloromethyl group, fluoromethyl group, bromomethyl |
| | group, dichloromethyl group, difluoromethyl group, |
| | dibromomethyl group, trichloromethyl group, |
| | trifluoromethyl group, bromodifluoromethyl group, |
| | 1,1,1-trifluoroethyl group, 1-chloroethyl group, |
| | 2-chloroethyl group, 1-bromoethyl group, |
| | pentafluoroethyl group or the like, |
| | preferably halo C1-6 alkyl group |
| Aryl group | Phenyl group, 1-naphthyl group, 2-naphthyl group, |
| | 1-indanyl group, 2-indanyl group, 1-indenyl group, |
| | 2-indenyl group or the like, |
| | preferably C6-10 aryl group |
| Unsaturated 5-membered | furan-2-yl group, furan-3-yl group, thiophene-2-yl |
| heterocyclic group | group, thiophene-3-yl group, pyrrole-2-yl group, |
| | pyrrole-3-yl group, oxazole-2-yl group, oxazole-4-yl |
| | group, oxazole-5-yl group, thiazole-2-yl group, |
| | thiazole-4-yl group, thiazole-5-yl group, |
| | isoxazol(e)-3-yl group, isoxazol(e)-4-yl group, |
| | isoxazol(e)-5-yl group, isothiazol(e)-3-yl group, |
| | isothiazol(e)-4-yl group, isothiazol(e)-5-yl group, |
| | imidazol(e)-2-yl group, imidazol(e)-4-yl group, |
| | imidazol(e)-5-yl group, pyrazol(e)-3-yl group, |
| | pyrazol(e)-4-yl group, pyrazole-5-yl group, |
| | 1,3,4-oxadiazole-2-yl group, 1,3,4-thiadiazole-2-yl |
| | group, 1,2,3-triazole-4-yl group, 1,2,4-triazole-3-yl |
| | group, 1,2,4-triazole-5-yl group or the like |
| Unsaturated 6-membered | pyridine-2-yl group, pyridine-3-yl group, |
| Heterocyclic group | pyridine-4-yl group, pyridazine-3-yl group, |
| | pyridazine-4-yl group, pyrazine-2-yl group, |
| | pyrimidine-2-yl group, pyrimidine-4-yl group, |
| | pyrimidine-5-yl group, 1,3,5-triazine-2-yl group, |
| | 1,2,4-triazine-3-yl group or the like |
| Saturated heterocyclic group | tetrahydrofuran-2-yl group, tetrahydropyran-4-yl |
| | group, piperidine-3-yl group, pyrrolidine-2-yl group, |
| | morpholino group, piperidino group, N-methyl |
| | piperazinyl group or the like |
| Monoalkyl amino group | Methyl amino group, ethyl amino group or the like |
| Monoaryl amino group | 1-naphthyl amino group, anilino group or the like |
| Dialkyl amino group | Dimethyl amino group, diethyl amino group or the |
| | like |
| Diaryl amino group | Diphenyl amino group, diindanyl amino group or the |
| | like |
| Alkyl sulfonyl amino group | Methyl sulfonyl amino group, ethyl sulfonyl amino |
| | group, n-propyl sulfonyl amino group, isopropyl |
| | sulfonyl amino group, n-butyl sulfonyl amino group, |
| | t-butyl sulfonyl amino group or the like |
| Aryl sulfonyl amino group | Phenyl sulfonyl amino group, indanyl sulfonyl |
| | amino group or the like |
| Heteroaryl sulfonyl amino group | Pyridine-3-yl sulfonyl amino group, furan-2-yl |
| | sulfonyl amino group or the like |
| Alkyl carbonyl amino group | Methyl carbonyl amino group, ethyl carbonyl amino |
| | group, n-propyl carbonyl amino group, isopropyl |
| | carbonyl amino group or the like |
| Alkoxycarbonyl amino group | Methoxycarbonyl amino group, ethoxycarbonyl |
| | amino group, n-propoxycarbonyl amino group, |
| | isopropoxycarbonyl amino group or the like |
| Bis(alkyl sulfonyl)amino group | Bis(methyl sulfonyl)amino group, bis(ethyl |
| | sulfonyl)amino group, (ethyl sulfonyl)(methyl |
| | sulfonyl)amino group, bis(n-propyl sulfonyl)amino |
| | group, bis(isopropyl sulfonyl)amino group, |
| | bis(n-butyl sulfonyl)amino group, bis(t-butyl |
| | sulfonyl)amino group or the like |
| N-unsubstituted or N-substituted | N-methyl iminomethyl group, 1-N-phenyl |
| iminoalkyl group | iminoethyl group, N-hydroxyiminomethyl group, |
| | N-methoxyiminomethyl group or the like, |
| | preferably N-unsubstituted or N-substituted imino |
| | C1-6 alkyl group |
| Aryl alkyl group | Benzyl group, phenethyl group or the like, |
| | preferably C6-10 aryl C1-6 alkyl group |
| Unsaturated 6-membered | Pyridine-2-yl methyl group, pyridine-3-yl methyl |
| heterocyclic alkyl group | group, 6-chloro-pyridine-3-yl methyl group, |
| | pyrimidine-2-yl methyl group or the like, |
| | preferably unsaturated 6-membered heterocyclic |
| | C1-6 alkyl group |
| Unsaturated 5-membered | Furan-2-yl-methyl group, thiophene-3-yl methyl |
| heterocyclic alkyl group | group, 1-methyl-pyrazol(e)-3-yl methyl group or |
| | the like, |
| | preferably unsaturated 5-membered heterocyclic |
| | C1-6 alkoxy group |
| Saturated heterocyclic alkyl group | Tetrahydrofuran-2-yl-methyl group, piperazine-3-yl |
| | methyl group, N-methyl-pyrrolidine-3-yl methyl |
| | group, morpholinomethyl group or the like |
| Alkoxy group | Methoxy group, ethoxy group, n-propoxy group, |
| | isopropoxy group, n-butoxy group, s-butoxy group, |
| | isobutoxy group, t-butoxy group or the like, |
| | preferably C1-6 alkoxy group |
| Haloalkoxy group | Chloromethoxy group, trifluoromethoxy group, |
| | 2,2,2-trifluoroethoxy group, 3-bromo-n-propoxy |
| | group, perfluoro-t-butoxy group or the like |
| | preferably C1-6 haloalkoxy group |
| Alkenyloxy group | Vinyloxy group, allyloxy group or the like, |
| | preferably C2-6 alkenyloxy group |
| Haloalkenyl group | 2,3-dichloroallyl group, 2-chloro-2-propenyl group, |
| | 2-chloromethyl-2-propenyl group, |
| | 4-bromo-2-butenyl group or the like, |
| | preferably C2-6 haloalkenyl group |
| Alkynyloxy group | Ethynyloxy group, propargyloxy group or the like, |
| | preferably C2-6 alkynyloxy group |
| Haloalkynyloxy group | 5-bromo-2-pentynyloxy group, |
| | 6-iodo-2-hexynyloxy group, 5,5,5-trifluoro- |
| | 2-pentynyloxy group, 3-chloropropargyloxy group |
| | or the like, |
| | preferably C2-6 haloalkynyloxy group |
| Aryloxy group | Phenoxy group, 1-naphthoxy group or the like, |
| | preferably C6-10 aryloxy group |
| Alkoxycarbonyloxy group | Methoxycarbonyloxy group, ethoxycarbonyloxy |
| | group, i-propoxycarbonyloxy group, |
| | t-butoxycarbonyloxy group or the like, |
| | preferably C1-6 alkoxycarbonyloxy group |
| Aryl alkyloxy group | Benzyloxy group, phenethyloxy group or the like, |
| | preferably C6-10 aryl C1-6 alkyloxy group |
| Heterocyclic oxy group | Pyridine-2-yloxy group, 3-oxazoline-2-yloxy |
| | group, pyrrolidine-2-yloxy group or the like |
| Alkyl thiocarbonyl group | Methyl thiocarbonyl group, ethyl thiocarbonyl |
| | group, n-propyl thiocarbonyl group, isopropyl |
| | thiocarbonyl group, n-butyl thiocarbonyl group, |
| | isobutyl thiocarbonyl group, s-butyl thiocarbonyl |
| | group, t-butyl thiocarbonyl group or the like, |
| | preferably C1-6 alkyl thiocarbonyl group |
| Alkoxy carbonyl group | Methoxycarbonyl group, ethoxycarbonyl group, |
| | n-propoxycarbonyl group, isopropoxycarbonyl |
| | group, n-butoxycarbonyl group, t-butoxycarbonyl |
| | group or the like, |
| | preferably C1-6 alkoxycarbonyl group |
| Unsubstituted or substituted amino | Aminocarbonyl group, dimethyl aminocarbonyl |
| carbonyl group | group, phenyl aminocarbonyl group or the like |
| Unsubstituted or substituted | Hydrazino group, N′-phenyl hydrazino group, |
| hydrazino group | N′-methoxycarbonyl hydrazino group, N′-acetyl |
| | hydrazino group, N′-methyl hydrazino group or the |
| | like |
| Unsubstituted or substituted | Hydrozinocarbonyl group, N′-methyl |
| hydrazino carbonyl group | hydrozinocarbonyl group, N′-phenyl |
| | hydrazinocarbonyl group or the like |
| Alkyl thio group | Methyl thio group, ethyl thio group, t-butyl thio |
| | group or the like, |
| | preferably C1-6 alkyl thio group |
| Alkenyl thio group | Vinyl thio group, allyl thio group or the like, |
| | preferably C2-6 alkeynyl thio group |
| Alkynyl thio group | Ethynyl thio group, propargyl thio group or the like, |
| | preferably C2-6 alkynyl thio group |
| Aryl thio group | Phenyl thio group, 4-chlorophenyl thio group or the |
| | like, |
| | preferably C6-10 aryl thio group |
| Heteroaryl thio group | Pyridine-2-yl thio group, pyridazine-3-yl thio group |
| | or the like, |
| Aryl alkyl thio group | Benzyl thio group, phenethyl thio group or the like, |
| | prererably C6-10 aryl C1-6 alkyl thio group |
| Alkyl sulfonyl group | Methyl sulfonyl group, ethyl sulfonyl group, t-butyl |
| | sulfonyl group or the like, |
| | preferably C1-6 alkyl sulfonyl group |
| Alkenyl sulfonyl group | Vinyl sulfonyl group, allyl sulfonyl group or the |
| | like, |
| | preferably C2-6 alkenyl sulfonyl group |
| Alkynyl sulfonyl group | Ethynyl sulfonyl group, propargyl sulfonyl group or |
| | the like, |
| | preferably C2-6 alkynyl sulfonyl group |
| Aryl sulfonyl group | Phenyl sulfonyl group, naphthyl sulfonyl group or |
| | the like, |
| | preferably C6-10 aryl sulfonyl group |
| Heteroaryl sulfonyl group | prydine-2-yl sufonyl group, prydine-3-yl sulfonyl |
| | group or the like |
| Aryl alkyl sulfonyl group | benzyl sulfonyl group, phenethyl sulfonyl group or |
| | the like, |
| | preferably C6-10 aryl C1-6 alkyl sulfonyl group |
| Acyl group | formyl group, acetyl group, propionyl, acryloyl |
| | group, cinnamoyl group, benzoyl group, |
| | pyridine-2-yl carbonyl group, cyclohexyl carbonyl |
| | group or the like, |
| | preferably C1-10 acyl group |
| Acyloxy group | formyloxy group, acetyloxy group, propionyloxy |
| | group, cinnamoyloxy group, benzoyloxy group, |
| | pyridine-2-yl carbonyloxy group, cyclohexyl |
| | carbonyloxy group or the like, |
| | preferably C1-10 acyloxy group |

### (R¹)

In the present invention, R¹ represents a halogen atom, hydroxyl group, mercapto group, NR^{a}₂ group (in the formula, R^{a} is as defined above), nitro group, or an organic group.

Examples of “halogen atom” include fluorine atom, chlorine atom, bromine atom, iodine atom and the like.

R^{a} of “NR^{a}₂ group” is as defined as R^{a} of the above described E. Examples of NR^{a}₂ include amino group, methyl amino group, dimethyl amino group, methyl ethyl amino group, vinyl amino group, allyl amino group, phenyl amino group, diphenyl amino group, benzyl amino group and the like.

“Organic group” indicates a general functional group having carbon atoms.

Examples of “organic group” include cyano group, an alkyl group, an alkenyl group, an alkynyl group, an alkoxy group, a cycloalkyl group, a cycloalkenyl group, an aryl group, an aryloxy group, an acyl group, an alkoxycarbonyl group, an alkyl thiocarbonyl group, an alkyl thio group, an alkyl sulfinyl group, an alkyl sulfonyl group, an aryl thio group, an aryl sulfinyl group, an aryl sulfonyl group, a substituted sulfoxyimino group and the like. Any of the examples of “organic group” may be substituted except nitro group.

The groups of the examples of “organic group” are defined as follows.

Examples of “alkyl group”, “alkenyl group”, “alkynyl group”, “alkoxy group”, “cycloalkyl group”, “cycloalkenyl group”, “aryl group”, “alkoxycarbonyl group”, “alkyl thio group”, and “acyl group” may be the same as the examples of the above described E and R^{a}.

Examples of “aryloxy group” include phenoxy group, 1-naphthyloxy group, 2-naphthyloxy group, azulenyloxy group, indenyloxy group, indanyloxy group, tetrolynyloxy group and the like. Among these examples, an aryloxy group having 6 to 14 carbon atoms is preferable. Within a chemically acceptable range, “aryloxy group” may have at least one or more types of substitutents, or one or more substituents selected from the substituents described in TABLE 1.

Examples of “alkyl thiocarbonyl group” include methyl thiocarbonyl group, ethyl thiocarbonyl group, n-propyl thiocarbonyl group, i-propyl thiocarbonyl group, n-butyl thiocarbonyl group, i-butyl thiocarbonyl group, s-butyl thiocarbonyl group, t-butyl thiocarbonyl group, n-pentyl thiocarbonyl group, n-hexyl thiocarbonyl group and the like. Among these examples, an alkyl thiocarbonyl group having 2 to 6 carbon atoms is preferable. Within a chemically acceptable range, “alkyl thiocarbonyl group” may have one or more types of substituents, or one or more substituents selected from the substituents described in TABLE 1.

Examples of “alkyl sulfinyl group” include methyl sulfinyl group, ethyl sulfinyl group, n-propyl sulfinyl group, i-propyl sulfinyl group, n-butyl sulfinyl group, 1-butyl sulfinyl group, s-butyl sulfinyl group, t-butyl sulfinyl group and the like. Among these examples, an alkyl sulfinyl group having 1 to 6 carbon atoms is preferable. Within a chemically acceptable range, “alkyl sulfinyl group” may have one or more types of substituents, or one or more substituents selected from the substituents described in TABLE 1.

Examples of “alkyl sulfonyl group” include methyl sulfonyl group, ethyl sulfonyl group, n-propyl sulfonyl group, i-propyl sulfonyl group, n-butyl sulfonyl group, i-butyl sulfonyl group, s-butyl sulfonyl group, t-butyl sulfonyl group and the like. Among these examples, an alkyl sulfonyl group having 1 to 6 carbon atoms is preferable. Within a chemically acceptable range, “alkyl sulfonyl group” may have one or more types of substituents, or one or more substituents selected from the substituents described in TABLE 1.

Examples of “aryl thio group” include phenyl thio group, 1-naphthyl thio group, 2-naphthyl thio group and the like. Among these examples, an aryl thio group having 6 to 14 carbon atoms is preferable. Within a chemically acceptable range, “aryl thio group” may have one or more types of substituents, or one or more substituents selected from the substituents described in TABLE 1.

Examples of “aryl sulfinyl group” include phenyl sulfinyl group, 1-naphthyl sulfinyl group, 2-naphthyl sulfinyl group and the like. Among these examples, an aryl sulfinyl group having 6 to 14 carbon atoms is preferable. Within a chemically acceptable range, “aryl sulfinyl group” may have one or more types of substituents, or one or more substituents selected from the substituents described in TABLE 1.

Examples of “aryl sulfonyl group” include phenyl sulfonyl group, 1-naphthyl sulfonyl group, 2-naphthyl sulfonyl group and the like. Among these examples, an aryl sulfonyl group having 6 to 14 carbon atoms is preferable. Within a chemically acceptable range, “aryl sulfonyl group” may have one or more types of substituents, or one or more substituents selected from the substituents described in TABLE 1.

“Substituted sulfoxyimino group” may be represented by formula —N═S(═O)(R′)(R″).

In this formula, R′ and R″ each independently represents the groups same as the examples of R² and R³ that are described hereinafter. “Substituted sulfoxyimino group” is preferably represented by formula —N═S(═O)R²R³.

In addition, R′ and R″ may bond together to form a ring.

Examples of “substituted sulfoxyimino group” include followings.

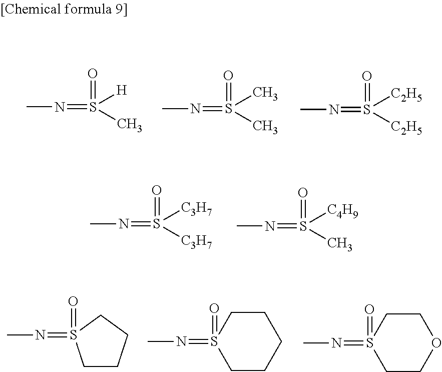

Among these examples, R¹ is preferably at least one selected from the group consisting of halogen atom, substituted or unsubstituted alkyl group and —N═S(═O)(R′)(R″), and more preferably a halogen atom or a haloalkyl group having 1 to 6 carbon atoms.

### (R², R³)

R² and R³ each independently represents an alkyl group, a cycloalkyl group, an alkenyl group, a alkynyl group, an aryl group, or a heterocyclic group.

Examples of “alkyl group”, “cycloalkyl group”, “alkenyl group”, “alkynyl group” and “aryl group” are the same as the examples of R¹. Within a chemically acceptable range, these groups may have one or more types of substitutents, or one or more substituents selected from the substituents described in TABLE 1.

“Heterocyclic group” indicates a 5- to 7-membered heteroaromatic ring, a 5- to 7-membered saturated heterocyclic ring or a 5- to 7-membered unsaturated heterocyclic ring, which have 1 to 4 hetero atoms selected from nitrogen atom, oxygen atom and sulfur atom other than carbon atom, or indicates a condensed heterocyclic ring in which benzene ring ad these heterocyclic rings are condensed. Examples of “heterocyclic group” included 2-furyl group, 3-furyl group, 2-thienyl group, 3-thienyl group, 2-oxazolyl group, 2-oxazolinyl group, 3-isoxazolyl group, 4-isoxazolyl group, 5-isoxazolyl group, 3-isoxazolinyl group, 2-thiazolyl group, 2-thiazolinyl group, 3-isothiazolyl group, 3-isothiazolinyl group, 2-pyranyl group, 4-tetrahydropyranyl group, 1-azetidinyl group, 2-azetidinyl group, 3-azetidinyl group, 2-pyrrolyl group, 2-pyrrolidinyl group, 2-imidazolyl group, 3-pyrazolyl group, 2-imidazolinyl group, 2-pyridyl group, 3-pyridyl group, 4-pyridyl group, 2-piperidyl group, piperidino group, 2-morpholinyl group, morpholino group, 2-piperazinyl group, 2-pyrimidinyl group, 3-pyridazinyl group, 2-pyrazinyl group and the like. Within a chemically acceptable range, “heterocylic group” may have one or more types of substituents, or one or more substitutents selected from the substitutents described in TABLE 1.

In addition, R² and R³ may bond together to than a 3- to 8-membered, substituted or unsubstituted heterocyclic group which may include 1 to 4 nitrogen atoms, oxygen atoms or sulfur atoms other than the sulfur atom in sulfoxyimino group. In this case, R² and R³ form a ring regardless of the functional groups of R² and R³ described above.

Examples of heterocyclic ring include thiophene ring, tetrahydrothiophene ring, thiopyran ring, tetrahydrothiopyran ring, 4-oxathiane ring, thiomorpholine ring, 1,4-dithiane ring, tetrahydrothiopyran-4-one ring and the like. Within a chemically acceptable range, these rings may have one or more types of substituents, or one or more substituents selected from the substituents described in TABLE 1.

### (Q)

Q represents a group selected from the following groups Q1 to Q8.

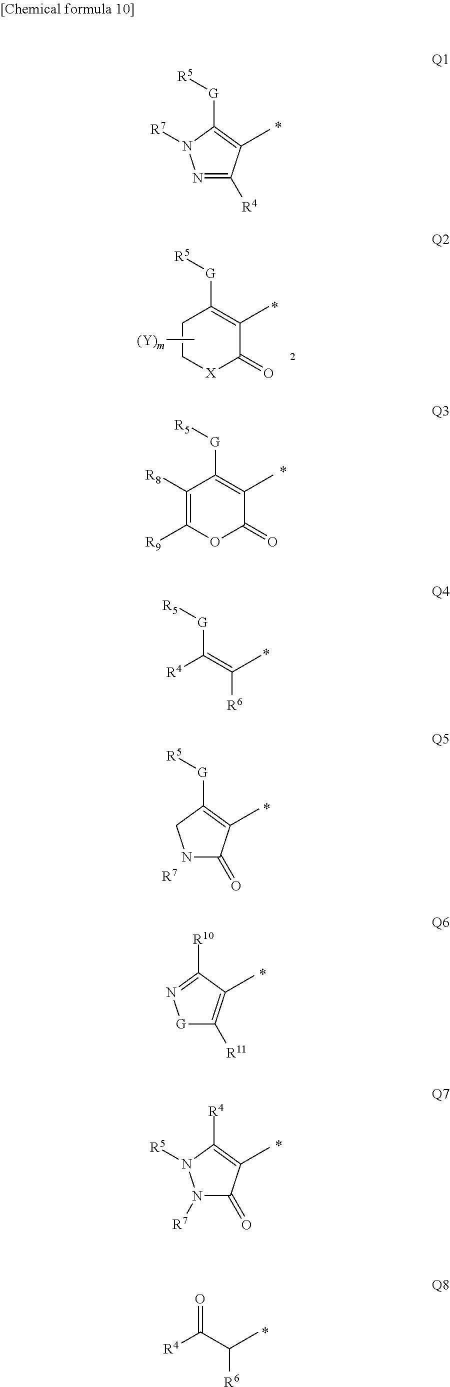

In the above formulas (Q1 to Q8), * represents binding site,

G represents oxygen atom, —S—, —S(O)—, —S(O)₂— or —NR^{b}—,

R^{b} in formula “—NR^{b}—” represents hydrogen atom or an organic group. Examples of “organic group” are the same as the examples of organic group of R¹ described above. Within a chemically acceptable range, “organic group” may have one or more types of substituents, or one or more substituents selected from the substituents described in TABLE 1.

Examples of “—NR^{b}—” include —NH—, —N(CH₃)—, —N(C₂H₅)—, —N(OCH₃)— and the like.

### (R⁴)

R⁴ represents hydrogen atom, an alkyl group, a cycloalkyl group or NR^{a}₂ group.

“Alkyl group”, “cycloalkyl group” and “NR^{a}₂ group” of R⁴ are the same as the examples of E and R¹ described above.

### (R⁵)

R⁵ represents hydrogen atom, an alkyl group, an alkenyl group, an alkynyl group, an aryl group, a heteroaryl group, an alkoxycarbonyl group, alkyl thiocarbonyl group, acyl group, R^{a}₂NC(O) group, an alkylsulfonyl group, an arylsulfonyl group or NR^{a}₂SO₂ group.

“Alkyl Group”, “Alkenyl Group”, “Alkynyl Group”, “Aryl Group”, “Alkoxycarbonyl group”, “alkyl thiocarbonyl group”, “acyl group”, “R^{a}₂NC(O) group”, “alkylsulfonyl group” and “arylsulfonyl group” of R⁵ are the same as the examples of E or R¹ described above. Within a chemically acceptable range, these groups may have one or more types of substituents, or one or more substituents selected from the substituents described in TABLE 1.

“Heteroaryl group” includes a 5- to 7-membered monocyclic or polycyclic heteroaromatic ring having 1 to 4 hetero atoms selected from nitrogen atom, oxygen atom and sulfur atom other than carbon atom, and a condensed ring in which benzene ring and a 5- to 7-membered heterocyclic ring having 1 to 4 hetero atoms selected from nitrogen atom, oxygen atom and sulfur atom are condensed. Examples of heteroaryl group include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl and the like.

Within a chemically acceptable range, “heteroaryl group” may have one or more types of substituents, or one or more substituents selected from the substituents described in TABLE 1.

R^{a} of “NR^{a}₂SO₂ group” is as defined above. Examples of “NR^{a}₂SO₂” include aminosulfonyl group, methyl aminosulfonyl group, ethyl aminosulfonyl group, allyl aminosulfonyl group, benzyl aminosulfonyl group, phenyl aminosulfonyl group, dimethyl aminosulfonyl group, phenyl methyl aminosulfonyl group and the like. Within a chemically acceptable range, “NR^{a}₂SO₂ group” may have one or more types of substituents, or one or more substituents selected from the substituents described in TABLE 1.

### (R⁶)

R⁶ represents cyano group, an acyl group, an alkoxycarbonyl group, C(R⁷¹)═NR⁷, or tetrazolyl group.

Examples of “Acyl Group” and “Alkoxycarbonyl Group” are the Same as the examples of E and R¹ described above. In addition, within a chemically acceptable range, these groups may have one or more types of substituents, or one or more substituents selected from the substituents described in TABLE 1.

In formula C(R⁷¹)═NR⁷, R⁷¹ represents hydrogen atom, an alkyl group, an aryl group, or a heteroaryl group.

Examples of “alkyl group”, “aryl group” and “heteroaryl group” are the same as the examples of E or R⁵ described above. Within a chemically acceptable range, these groups may have one or more types of substituents, or one or more substituents selected from the substituents described in TABLE 1.

In addition, in formula C(R⁷¹)═NR⁷, R⁷ represents hydrogen atom, an alkyl group or an alkoxy group. Examples of “alkyl group” and “alkoxy group” are the same as the examples of E described above. Within a chemically acceptable range, these groups may have one or more types of substituents, or one or more substituents selected from the substituents described in TABLE 1.

### (R⁸, R⁹)

R⁸ and R⁹ each independently represents hydrogen atom or an alkyl group.

Examples of “Alkyl Group” are the Same as the Examples of Alkyl Group of E described above. Within a chemically acceptable range, “alkyl group” may have one or more types of substituents, or one or more substituents selected from the substituents described in TABLE 1.

### (R¹⁰, R¹¹)

R¹⁰ and R¹¹ each independently represents hydrogen atom, an alkyl group or a cycloalkyl group.

Examples of “alkyl group” and “cycloalkyl group” are the same as the examples of E or R¹ described above. Within a chemically acceptable range, these groups may have one or more types of substituents, or one or more substituents selected from the substituents described in TABLE 1.

### (X)

X represents —C(R¹²)(R¹³)— or —N(R¹²)—.

R¹² and R¹³ each independently represents hydrogen group or an alkyl group.

Examples of “alkyl group” may be the same as the examples of alkyl group of E described above. Within a chemically acceptable range, “alkyl group” may have one or more types of substituents, or one or more substituents selected from the substituents described in TABLE 1.

### (Y)

Y represents oxo group (═O), an alkyl group, an alkoxy group, an acyl group or an alkoxycarbonyl group.

Examples of “alkyl group”, “alkoxy group”, “acyl group” and “alkoxycarbonyl group” may be the same as the examples of E described above. Within a chemically acceptable range, these groups may have one or more types of substituents, or one or more substituents selected from the substituents described in TABLE 1.

m represents an integer of 0 to 4.

When m is 2 or more, the numerous Y may be the same or different from each other, and Y may bond with each other to form a ring regardless of the above described substituents. In addition, Y and R¹² of X may bond together to form a ring regardless of the above described substituents.

As shown in below, the examples of the above described ring include a 3- to 8-membered spiro ring represented by the following formula (Q2-1), a 3- to 8-membered condensed ring represented the following formula (Q2-2) and a 3- to 8-membered bridge ring represented by the following formula (Q2-3) or the like, which form a C1-6 alkylene chain having 0 to 3 nitrogen atoms, oxygen atoms or sulfur atoms. In addition, the examples of the ring which formed by bonding Y and Y may be shown by formulas (Q2-4) to (Q2-8), and the examples of the ring which formed by bonding Y and R¹² may be shown by formulas (Q2-9), (Q2-10).

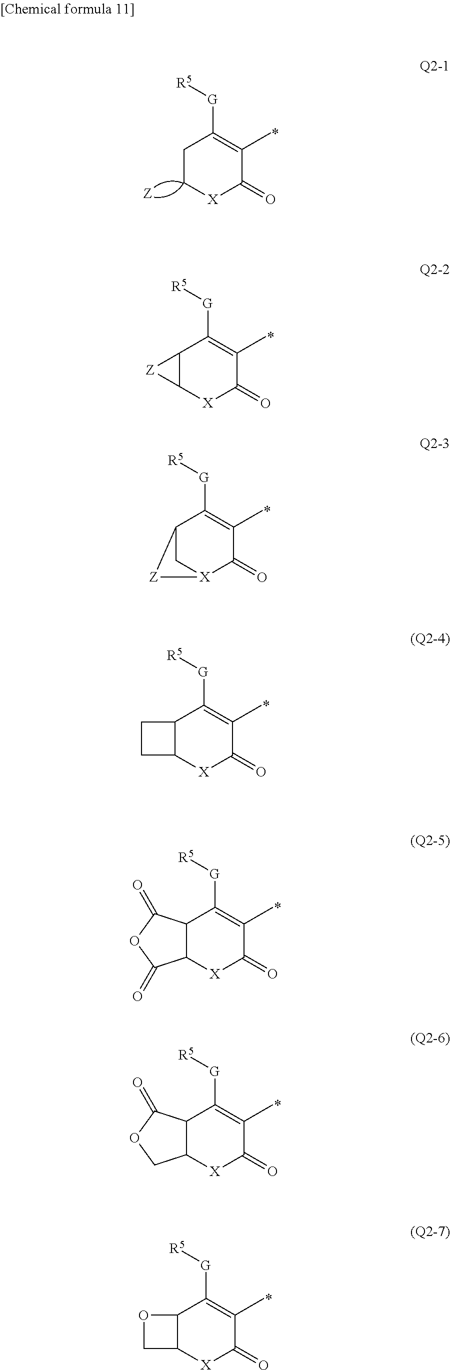

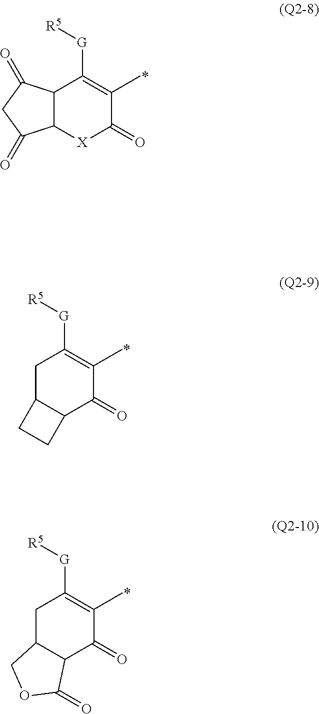

(In the formula, G, R⁵, X are as defined above, Z represents a C1-6 alkylene chain having 0 to 3 nitrogen atoms, oxygen atoms and/or sulfur atoms).

From the point of view of having an excellent herbicide activity, the benzoyl derivative of the present invention is preferably a compound represented by the following formula (I-a)

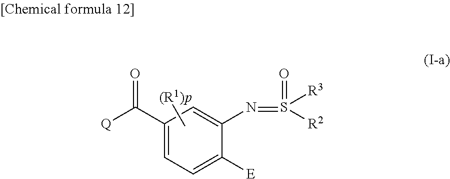

(In the formula, E, R¹ to R³, p, and Q are as defined above), more preferably a compound represented by the following formula (I-b)

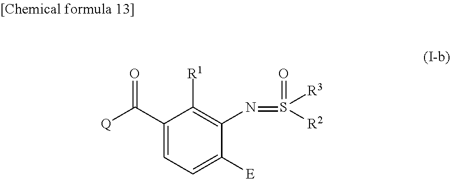

(In the formula, E, R¹ to R³, and Q are as defined above).

In addition, the benzoyl derivative and salt thereof of the present invention is more preferably a compound represented by formula (I-b), wherein R¹ represents a halogen atom, a substituted or unsubstituted alkyl group or a group represented by formula —N═S(═O)R²R³ (in the formula, R², R³ are as defined above).

Examples of the salt of the compound (I) of the present invention include a salt of alkali metal such as lithium, sodium, potassium or the like; a salt of alkali earth metal such as calcium, magnesium, or the like; a salt of transition metal such as iron, copper or the like; a salt of organic base such as ammonia, triethylamine, tributylamine, pyridine, hydrazine or the like.

Compound (I) of the present invention may have stereoisomer or tautomer based on asymmetric carbon or double bond. All of these isomers and mixture thereof are included in the technical scope of the present invention.

Optical isomer is possible for the compound of the present invention and the compound also may have numerous tautomers. All of these isomers are included in the scope of the present invention.

The structure of the compound of the present invention may be determined by NMR spectrum, IR spectrum, MS spectrum or the like.

### Examples of the Compound of the Present Invention

The representative examples of the compound of the present invention are shown in the following tables. However, the compound of the present invention is not limited by these compounds.

In addition, the abbreviations described in the tables have the meanings as defined below.

Me: methyl, Et: ethyl, Pr: propyl, Ph: phenyl, n: normal, i: iso,

c: cyclo, Tosyl: p-toluensulfonyl.

TABLE 2
Example of Compound (1)

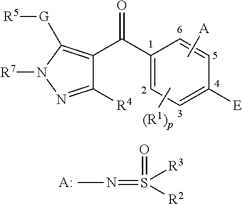

| | | | Position | | | | | | |
|---|---|---|---|---|---|---|---|---|---|
| No. | E | (R¹)p | of A | R² | R³ | R⁴ | R⁵ | R⁷ | G |
| 1 | H | 2-Cl | 3 | Me | Me | H | H | Me | O |
| 2 | H | 2-Cl | 3 | Et | Et | H | CF₃ | Et | O |
| 3 | H | 2-Cl | 3 | n-Pr | n-Pr | H | H | i-Pr | O |
| 4 | H | 2-Cl | 3 | Me | c-Pr | Me | Me | Me | O |
| 5 | CN | 2-Cl | 3 | Me | CF₃ | H | Ph | CF₃ | S |
| 6 | Me | 2-Cl | 3 | Me | CH₃═CHCH₂— | H | PhCH₂ | H | O |
| 7 | Me | 2-Cl | 3 | Me | CH≡CCH₂— | H | Tosyl | H | O |
| 8 | Me | 2-Me₂N | 3 | Me | Ph | H | MeC(O) | MeO | O |
| 9 | Me | 2-NH₂ | 3 | Me | 2-Thienyl | c-Pr | PhC(O) | EtO | O |
| 10 | MeS | 5-Cl, 6-F | 3 | Me | 2-Furyl | H | CH₂═CH | H | S(O) |
| 11 | CF₃ | 2-Cl | 3 | Me | 2-Pyridyl | H | CH≡C | Me | O |
| 12 | CF₃ | 2-Cl | 3 | Me | 4-Piperidyl | H | 2-Pyridyl | Et | O |
| 13 | CF₃ | 2-Cl | 3 | —CH₂CH₂CH₂— | | H | H | i-Pr | O |
| 14 | CF₃ | 2-Cl | 3 | —CH₂CH(Me)CH₂— | | NH₂ | MeSO₂ | CF₃ | O |
| 15 | CF₃ | 2-Cl | 3 | —CH₂CH₂CH₂CH₂— | | H | PhSO₂ | Me | S(O)₂ |
| 16 | MeO | 2-Cl | 3 | —CH₂CH₂CH₂CH₂CH₂— | | H | N(Me)₂SO₂ | H | O |
| 17 | MeO | 2-Cl | 3 | —CH₂CH₂—O—CH₂CH₂— | | H | H | H | O |
| 18 | MeO | 2-Cl | 3 | —CH₂CH₂—C(O)—CH₂CH₂— | | H | CF₃ | MeO | O |
| 19 | MeO | 2-Cl | 3 | —CH₂CH₂CH(OEt)—CH₂CH₂— | | N(Me)₂ | H | EtO | O |
| 20 | MeO | 2-Cl | 3 | —CH₂CH₂—N(Me)—CH₂CH₂— | | H | Me | H | NH |
| 21 | Ph | 2-Cl | 3 | Me | Me | H | Ph | Me | O |
| 22 | Ph | 2-Cl | 3 | Et | Et | H | PhCH₂ | CF₃ | O |
| 23 | Ph | 2-Cl | 3 | n-Pr | n-Pr | H | Tosyl | i-Pr | O |
| 24 | Ph | 2-Cl | 3 | Me | c-Pr | Me | MeC(O) | Me | O |
| 25 | Ph | 2-Cl | 3 | Me | CF₃ | H | PhC(O) | Me | N(Me) |
| 26 | NH₂ | 2-Cl | 3 | Me | CH₂═CHCH₂— | H | CH₂═CH | H | O |
| 27 | NH₂ | 2-Me | 3 | Me | CH≡CCH₂— | H | CH≡C | H | O |
| 28 | NH₂ | 2-Me | 3 | Me | Ph | H | 2-Pyridyl | MeO | O |
| 29 | NH₂ | 2-Me | 3 | Me | 2-Thienyl | c-Pr | H | EtO | O |
| 30 | NH₂ | 2-Me | 3 | Me | 2-Furyl | H | MeSO₂ | H | S |
| 31 | N(Me)2 | 2-Cl | 3 | Me | 2-Pyridyl | H | PhSO₂ | Me | O |
| 32 | N(Me)2 | 2-Me | 3 | Me | 4-Piperidyl | H | N(Me)₂SO₂ | Et | O |
| 33 | N(Me)2 | 2-Me | 3 | —CH₂CH₂CH₂— | | H | H | i-Pr | O |
| 34 | N(Me)2 | 2-Me | 3 | —CH₂CH(Me)CH₂— | | NH₂ | CF₃ | Me | O |
| 35 | N(Me)2 | 2-Me | 3 | —CH₂CH₂CH₂CH₂— | | H | H | Me | S(O) |
| 36 | HN(Me)C(O) | 2-Cl | 3 | —CH₂CH₂CH₂CH₂CH₂— | | H | Me | H | O |
| 37 | HN(Me)C(O) | 2-CF₃ | 3 | —CH₂CH₂—O—CH₂CH₂— | | H | Ph | H | O |
| 38 | HN(Me)C(O) | 2-CF₃ | 3 | —CH₂CH₂—C(O)—CH₂CH₂— | | H | PhCH₂ | MeO | O |
| 39 | HN(Me)C(O) | 2-CF₃ | 3 | —CH₂CH₂CH(OEt)CH₂CH₂— | | N(Me)₂ | Tosyl | EtO | O |
| 40 | HN(Me)C(O) | 2-CF₃ | 3 | —CH₂CH₂—N(Me)—CH₂CH₂— | | H | MeSC(O) | H | S(O)₂ |
| 41 | MeOC(O) | 2-Cl | 3 | Me | Me | H | PhC(O) | Me | O |
| 42 | MeOC(O) | 2-CF₃ | 3 | Et | Et | H | CH₂═CH | CF₃ | O |
| 43 | MeOC(O) | 2-CF₃ | 3 | Me | n-Pr | H | CH≡C | i-Pr | O |
| 44 | MeOC(O) | 2-CF₃ | 3 | Me | c-Pr | CF₃ | 2-Pyridyl | Me | O |
| 45 | MeOC(O) | 2-CF₃ | 3 | Me | CF₃ | H | H | Me | NH |
| 46 | c-Pentyloxy | 2-Cl | 3 | Me | CH₂═CHCH₂— | H | MeSO₂ | H | O |
| 47 | c-Pentyloxy | 2-OH | 3 | Me | CH≡CCH₂— | H | PhSO₂ | H | O |
| 48 | c-Pentyloxy | 2-OH | 3 | Me | Ph | H | N(Me)₂SO₂ | MeO | O |
| 49 | c-Pentyloxy | 2-OH | 3 | Me | 2-Thienyl | c-Pr | H | EtO | O |
| 50 | c-Pentyloxy | 2-OH | 3 | Me | 2-Furyl | H | CF₃ | H | N(Me) |
| 51 | PhC(O) | 2-Cl | 5 | Me | Me | H | H | Me | O |
| 52 | 2-Pyridyl | 2-MeO | 5 | Et | Et | H | CF3 | Et | O |
| 53 | H | 2-MeO | 5 | n-Pr | n-Pr | H | EtOC(O) | i-Pr | O |
| 54 | H | 2-MeO | 5 | Me | c-Pr | Me | Me | CF₃ | O |
| 55 | H | 2-MeO | 5 | Me | CF₃ | H | Ph | Me | S |
| 56 | Me | 2-Cl | 5 | Me | CH₂═CHCH₂— | H | PhCH₂ | H | O |
| 57 | Me | 2-CF₃O | 5 | Me | CH≡CCH₂— | H | Tosyl | H | O |
| 58 | Me | 2-CF₃O | 5 | Me | Ph | H | MeC(O) | MeO | O |
| 59 | Me | 2-CF₃O | 5 | Me | 2-Thienyl | c-Pr | PhC(O) | EtO | O |
| 60 | Me | 2-CF₃O | 5 | Me | 2-Furyl | H | CH₂═CH | H | S(O) |
| 61 | CF₃ | 2-Cl | 5 | Me | 2-Pyridyl | H | CH≡C | Me | O |
| 62 | CF₃ | 2-CF₃O | 5 | Me | 4-Piperidyl | H | 2-Pyridyl | Et | O |
| 63 | CF₃ | 2-CF₃O | 5 | —CH₂CH₂CH₂— | | H | H | i-Pr | |
| 64 | CF₃ | 2-CF₃O | 5 | —CH₂CH(Me)CH₂— | | NH₂ | MeSO₂ | Me | O |
| 65 | CF₃ | 2-CF₃O | 5 | —CH₂CH₂CH₂CH₂— | | H | PhSO₂ | Me | S(O)₂ |
| 66 | MeO | 2-Cl | 5 | —CH₂CH₂CH₂CH₂CH₂— | | H | N(Me)₂SO₂ | H | O |
| 67 | MeO | 2-SH | 5 | —CH₂CH₂—O—CH₂CH₂— | | H | H | H | O |
| 68 | MeO | 2-SH | 5 | —CH₂CH₂—C(O)—CH₂CH₂— | | H | CF₃ | MeO | O |
| 69 | MeO | 2-SH | 5 | —CH₂CH₂CH(OEt)CH₂CH₂— | | N(Me)₂ | H | EtO | O |
| 70 | MeO | 2-SH | 5 | —CH₂CH₂—N(Me)—CH₂CH₂— | | H | Me | H | NH |
| 71 | Ph | 2-Cl | 6 | Me | Me | H | Ph | Me | O |
| 72 | Ph | 2-MeS | 6 | Et | Et | H | PhCH₂ | CF₃ | O |
| 73 | Ph | 2-MeS | 6 | n-Pr | n-Pr | H | Tosyl | i-Pr | O |
| 44 | Ph | 2-MeS | 6 | Me | c-Pr | CF₃ | MeC(O) | Me | O |
| 75 | Ph | 2-MeS | 6 | Me | CF₃ | H | PhC(O) | Me | N(Me) |
| 76 | NH₂ | 2-Cl | 6 | Me | CH₂═CHCH₂— | H | CH₂═CH | H | O |
| 77 | NH₂ | 2-MeS(O)₂ | 6 | Me | CH≡CCH₂— | H | CH≡C | H | O |
| 78 | NH₂ | 2-MeS(O)₂ | 6 | Me | Ph | H | 2-Pyridyl | MeO | O |
| 79 | NH₂ | 2-MeS(O)₂ | 6 | Me | 2-Thienyl | c-Pr | H | EtO | O |
| 80 | NH₂ | 2-MeS(O)₂ | 6 | Me | 2-Furyl | H | MeSO₂ | H | S |
| 81 | N(Me)2 | 2-Cl | 6 | Me | 2-Pyridyl | H | PhSO₂ | Me | O |
| 82 | N(Me)2 | 2-NO₂ | 6 | Me | 4-Piperidyl | H | N(Me)₂SO₂ | Et | O |
| 83 | N(Me)2 | 2-NO₂ | 6 | —CH₂CH₂CH₂— | | H | H | i-Pr | O |
| 84 | N(Me)2 | 2-NO₂ | 6 | —CH₂CH(Me)CH₂— | | NH2 | CF₃ | Me | O |
| 85 | N(Me)2 | 2-NO₂ | 6 | —CH₂CH₂CH₂CH₂— | | H | H | Me | S(O) |
| 86 | HN(Me)C(O) | 2-Cl | 6 | —CH₂CH₂CH₂CH₂CH₂— | | H | Me | H | O |
| 87 | HN(Me)C(O) | 2,5-diMe | 6 | —CH₂CH₂—O—CH₂CH₂— | | H | Ph | H | O |
| 88 | HN(Me)C(O) | 2,5-diMe | 6 | —CH₂CH₂—C(O)—CH₂CH₂— | | H | PhCH₂ | MeO | O |
| 89 | HN(Me)C(O) | 2,5-diMe | 6 | —CH₂CH₂CH(OEt)CH₂CH₂— | | N(Me)₂ | Tosyl | EtO | O |
| 90 | HN(Me)C(O) | 2,5-diMe | 6 | —CH₂CH₂—N(Me)—CH₂CH₂— | | H | MeC(O) | H | S(O)₂ |
| 91 | MeOC(O) | 2-Cl | 3 | Me | Me | H | PhC(O) | Me | O |
| 92 | MeOC(O) | 2,5-diCl | 3 | Et | Et | H | CH₂═CH | CF₃ | O |
| 93 | MeOC(O) | 2,5-diCl | 3 | n-Pr | n-Pr | H | CH≡C | i-Pr | O |
| 94 | MeOC(O) | 2,5-diCl | 3 | Me | c-Pr | Me | 2-Pyridyl | Me | O |
| 95 | MeOC(O) | 2,5-diCl | 3 | Me | CF₃ | H | H | Me | NH |
| 96 | c-Pentyloxy | 2-Cl | 3 | Me | CH₂═CHCH₂— | H | MeSO₂ | H | O |
| 97 | c-Pentyloxy | — | 3 | Me | CH≡CCH₂— | H | PhSO₂ | H | O |
| 98 | c-Pentyloxy | — | 3 | Me | Ph | H | N(Me)₂SO₂ | MeO | O |
| 99 | c-Pentyloxy | — | 3 | Me | 2-Thienyl | c-Pr | H | EtO | O |
| 100 | c-Pentyloxy | — | 3 | Me | 2-Furyl | H | CF₃ | H | N(Me) |

TABLE 3

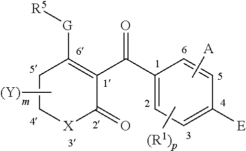

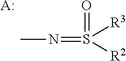

Exanple of Compound (2)

| | | | Position | | | | | | |
|---|---|---|---|---|---|---|---|---|---|
| No. | E | (R¹)ₚ | of A | R² | R³ | X | R⁵ | (Y)m | G |
| 1 | H | 2-Cl | 3 | Me | Me | CH₂ | H | — | O |
| 2 | H | 2-Cl | 3 | Et | Et | CH₂ | CF₃ | — | O |
| 3 | H | 2-Cl | 3 | n-Pr | n-Pr | CH₂ | H | — | O |
| 4 | H | 2-Cl | 3 | Me | c-Pr | CH₂ | Me | — | O |
| 5 | H | 2-Cl | 3 | Me | CF₃ | CH₂ | Ph | 5′-Me | S |
| 6 | iPrS | 2-Cl | 3 | Me | CH₂═CHCH₂— | CH(Me) | PhCH₂ | — | O |
| 7 | PhC(O) | 2-Cl | 3 | Me | CH≡CCH₂— | C(Me)₂ | Tosyl | — | O |
| 8 | 3-Furyl | 2-Cl | 3 | Me | Ph | NH | MeC(O) | — | O |
| 9 | Me | 2-Cl | 3 | Me | 2-Thienyl | N(Me) | PhC(O) | — | O |
| 10 | Me | 2-Cl | 3 | Me | 2-Furyl | CH₂ | CH═CH | — | S(O) |
| 11 | CF₃ | 2-Cl | 3 | Me | 2-Pyridyl | CH₂ | CH≡C | 5′-oxo | O |
| 12 | CF₃ | 2-Cl | 3 | Me | 4-Piperidyl | CH₂ | 2-Pyridyl | 5′-CF₃ | O |
| 13 | CF₃ | 2-Cl | 3 | —CH₂CH₂CH₂— | | CH₂ | CH₂ | 5′-MeO | O |
| 14 | CF₃ | 2-Cl | 3 | —CH₂CH(Me)CH₂— | | CH₂ | CH₂ | 5′,5′-diMe | O |
| 15 | CF₃ | 2-Cl | 3 | —CH₂CH₂CH₂CH₂— | | CH₂ | PhSO₂ | 5′-MeC(O) | S(O)₂ |
| 16 | MeO | 2-Cl | 3 | —CH₂CH₂CH₂CH₂CH₂— | | CH(Me) | Me₂NCO | 5′-PhC(O) | O |
| 17 | MeO | 2-Cl | 3 | —CH₂CH₂—O—CH₂CH₂— | | C(Me)₂ | H | 5′-MeOC(O) | O |
| 18 | MeO | 2-Cl | 3 | —CH₂CH₂—C(O)—CH₂CH₂— | | NH | CF₃ | 4′-oxo | O |
| 19 | MeO | 2-Cl | 3 | —CH₂CH₂CH(OEt)CH₂CH₂— | | N(Me) | H | 4′-Me | O |
| 20 | MeO | 2-Cl | 3 | —CH₂CH₂—N(Me)—CH₂CH₂— | | CH₂ | Me | 4′-MeO | NH |
| 21 | Ph | 2-Cl | 3 | Me | Me | CH₂ | EtSC(O) | 4′,4′-diMe | O |
| 22 | Ph | 2-Cl | 3 | Et | Et | CH₂ | tBu | 4′-MeC(O) | O |
| 23 | Ph | 2-Cl | 3 | n-Pr | n-Pr | CH₂ | PhC(O) | 4′-PhC(O) | O |
| 24 | Ph | 2-Cl | 3 | Me | c-Pr | CH₂ | MeOC(O) | 4′-MeOC(O) | O |
| 25 | Ph | 2-Cl | 3 | Me | CF₃ | CH₂ | PhC(O) | — | N(Me) |
| 26 | NH₂ | 2-Cl | 3 | Me | CH₂═CHCH₂— | CH(Me) | CH₂═CH | — | O |
| 27 | NH₂ | 2-NH₂ | 3 | Me | CH≡CCH₂— | C(Me)₂ | CH≡C | — | O |
| 28 | NH₂ | 2-Me | 3 | Me | Ph | NH | 2-Pyridyl | — | O |
| 29 | NH₂ | 2-Me | 3 | Me | 2-Thienyl | N(Me) | MeSC(O) | 5-Me | O |
| 30 | NH₂ | 2-Me | 3 | Me | 2-Furyl | CH₂ | MeSO₂ | — | S |
| 31 | N(Me)2 | 2-Cl | 3 | Me | 2-Pyridyl | CH₂ | PhSO₂ | — | O |
| 32 | N(Me)2 | 2-Me | 3 | Me | 4-Piperidyl | CH₂ | N(Me)₂SO₂ | — | O |
| 33 | N(Me)2 | 2-Me | 3 | —CH₂CH₂CH₂— | | CH₂ | H | — | O |
| 34 | N(Me)2 | 2-Me | 3 | —CH₂CH(Me)CH₂— | | CH₂ | CF₃ | — | O |
| 35 | N(Me)2 | 2-Me | 3 | —CH₂CH₂CH₂CH₂— | | CH₂ | H | 5′-oxo | S(O) |
| 36 | HN (Me) C(O) | 2-Cl | 3 | —CH₂CH₂CH₂CH₂CH₂— | | CH(Me) | Me | 5′-CF₃ | O |
| 37 | HN (Me) C(O) | 2-CF₃ | 3 | —CH₂CH₂—O—CH₂CH₂— | | C(Me)₂ | Ph | 5′-MeO | O |
| 38 | HN (Me) C(O) | 2-CF₃ | 3 | —CH₂CH₂—C(O)—CH₂CH₂— | | NH | PhCH₂ | 5′,5′-diMe | O |
| 39 | HN (Me) C(O) | 2-CF₃ | 3 | —CH₂CH₂CH(OEt)CH₂CH₂— | | N(Me) | Tosyl | 5′-MeC(O) | O |
| 40 | HN(Me) C(O) | 2-CF₃ | 3 | —CH₂CH₂—N(Me)—CH₂CH₂— | | CH₂ | MeC(O) | 5′-PhC(O) | S(O)₂ |
| 41 | MeOC(O) | 2-Cl | 3 | Me | Me | CH₂ | PhC(O) | 5′-MeOC(O) | O |
| 42 | MeOC(O) | 2-CF₃ | 3 | Et | Et | CH₂ | CH═CH | 4′-oxo' | O |
| 43 | MeOC(O) | 2-CF₃ | 3 | n-Pr | n-Pr | CH₂ | CH≡C | 4′-Me | O |
| 44 | MeOC(O) | 2-CF₃ | 3 | Me | c-Pr | CH₂ | 2-Pyridyl | 4′-MeO | O |
| 45 | MeOC(O) | 2-CF₃ | 3 | Me | CF₃ | CH₂ | H | 4′,4′-diMe | NH |
| 46 | c-Pentyloxy | 2-Cl | 3 | Me | CH₂═CHCH₂— | CH(Me) | MeSO₂ | 4′-MeC(O) | O |
| 47 | c-Pentyloxy | 2-OH | 3 | Me | CH≡CCH₂— | C(Me)₂ | PhSO₂ | 4′-PhC(O) | O |
| 48 | c-Pentyloxy | 2-OH | 3 | Me | Ph | NH | N(Me)₂SO₂ | 4′-MeOC(O) | O |
| 49 | c-Pentyloxy | 2-OH | 3 | Me | 2-Thienyl | N(Me) | H | — | O |
| 50 | c-Pentyloxy | 2-OH | 3 | Me | 2-Furyl | CH₂ | CF₃ | — | N(Me) |
| 51 | H | 2-Cl | 5 | Me | Me | CH₂ | H | — | O |
| 52 | H | 2-MeO | 5 | Et | Et | CH₂ | CF₃ | — | O |
| 53 | H | 2-MeO | 5 | n-Pr | n-Pr | CH₂ | H | 5′-Me | O |
| 54 | H | 2-MeO | 5 | Me | c-Pr | CH₂ | Me | — | O |
| 55 | H | 2-MeO | 5 | Me | CF₃ | CH₂ | Ph | — | S |
| 56 | Me | 2-Cl | 5 | Me | CH₂═CHCH₂— | CH(Me) | PhCH₂ | — | O |
| 57 | Me | 2-CF₃O | 5 | Me | CH≡CCH₂— | C(Me)₂ | Tosyl | 5′-oxo | O |
| 58 | Me | 2-CF₃O | 5 | Me | Ph | NH | MeC(O) | 5′-CF₃ | O |
| 59 | Me | 2-CF₃O | 5 | Me | 2-Thienyl | N(Me) | PhC(O) | 5′-MeO | O |
| 60 | Me | 2-CF₃O | 5 | Me | 2-Furyl | CH₂ | CH═CH | 5′,5′-diMe | S(O) |
| 61 | CF₃ | 2-Cl | 5 | Me | 2-Pyridyl | CH₂ | CH≡C | 5′-MeC(O) | O |
| 62 | CF₃ | 2-CF₃O | 5 | Me | 4-Piperidyl | CH₂ | 2-Pyridyl | 5′-PhC(O) | O |
| 63 | CF₃ | 2-CF₃O | 5 | —CH₂CH₂CH₂— | | CH₂ | H | 5′-MeOC(O) | O |
| 64 | CF₃ | 2-CF₃O | 5 | —CH₂CH(Me)CH₂— | | CH₂ | MeSO₂ | 4′-oxo′ | O |
| 65 | CF₃ | 2-CF₃O | 5 | —CH₂CH₂CH₂CH₂— | | CH₂ | PhSO₂ | 4′-Me | S(O)₂ |
| 66 | MeO | 2-Cl | 5 | —CH₂CH₂CH₂CH₂CH₂— | | CH(Me) | N(Me)₂SO₂ | 4′-MeO | O |
| 67 | MeO | 2-SH | 5 | —CH₂CH₂—O—CH₂CH₂— | | C(Me)₂ | H | 4′,4′-diMe | O |
| 68 | MeO | 2-SH | 5 | —CH₂CH₂—C(O)—CH₂CH₂— | | NH | CF₃ | 4′-MeC(O) | O |
| 69 | MeO | 2-SH | 5 | —CH₂CH₂CH(OEt)CH₂CH₂— | | N(Me) | H | 4′-PhC(O) | O |
| 70 | MeO | 2-SH | 5 | —CH₂CH₂—N(Me)—CH₂CH₂— | | CH₂ | Me | 4′-MeOC(O) | NH |
| 71 | Ph | 2-Cl | 6 | Me | Me | CH₂ | Ph | — | O |
| 72 | Ph | 2-MeS | 6 | Et | Et | CH₂ | PhCH₂ | — | O |
| 73 | Ph | 2-MeS | 6 | n-Pr | n-Pr | CH₂ | Tosyl | — | O |
| 74 | Ph | 2-MeS | 6 | Me | c-Pr | CH₂ | MeC(O) | | O |
| 75 | Ph | 2-MeS | 6 | Me | CF₃ | CH₂ | PhC(O) | — | N(Me) |
| 76 | NH₂ | 2-Cl | 6 | Me | CH₂═CHCH₂— | CH(Me) | CH═CH | 5′-Me | O |
| 77 | NH₂ | 2-MeS(O)₂ | 6 | Me | CH≡CCH₂— | C(Me)₂ | CH≡C | — | O |
| 78 | NH₂ | 2-MeS(O)₂ | 6 | Me | Ph | NH | 2-Pyridyl | — | O |
| 79 | NH₂ | 2-MeS(O)₂ | 6 | Me | 2-Thienyl | N(Me) | H | — | O |
| 80 | NH₂ | 2-MeS(O)₂ | 6 | Me | 2-Furyl | CH₂ | MeSO₂ | — | S |
| 81 | N(Me)2 | 2-Cl | 6 | Me | 2-Pyridyl | CH₂ | PhSO₂ | 5′-oxo | O |
| 82 | N(Me)2 | 2-NO₂ | 6 | Me | 4-Piperidyl | CH₂ | N(Me)₂SO₂ | 5′-CF₃ | O |
| 83 | N(Me)2 | 2-NO₂ | 6 | —CH₂CH₂CH₂— | | CH₂ | H | 5′-MeO | O |
| 84 | N(Me)2 | 2-NO₂ | 6 | —CH₂CH(Me)CH₂— | | CH₂ | CF₃ | 5′,5′-diMe | O |
| 85 | N(Me)2 | 2-NO₂ | 6 | —CH₂CH₂CH₂CH₂— | | CH₂ | H | 5′-MeC(O) | S(O) |
| 86 | HN(Me) C(O) | 2-Cl | 6 | —CH₂CH₂CH₂CH₂CH₂— | | CH(Me) | Me | 5′-PhC(O) | O |
| 87 | HN (Me) C(O) | 2,5-diMe | 6 | —CH₂CH₂—O—CH₂CH₂— | | C(Me)₂ | Ph | 5′-MeOC(O) | O |
| 88 | HN (Me) C(O) | 2,5-diMe | 6 | —CH₂CH₂—C(O)—CH₂CH₂— | | NH | PhCH₂ | 4′-oxo′ | O |
| 89 | HN (Me) C(O) | 2,5-diMe | 6 | —CH₂CH₂CH(OEt)CH₂CH₂— | | N(Me) | Tosyl | 4′-Me | O |
| 90 | HN (Me) C(O) | 2,5-diMe | 6 | —CH₂CH₂—N(Me)—CH₂CH₂— | | CH₂ | MeC(O) | 4′-MeO | S(O)₂ |
| 91 | MeOC(O) | 2-Cl | 3 | Me | Me | CH₂ | PhC(O) | 4′,4′-diMe | O |
| 92 | MeOC(O) | 2,5-diCl | 3 | Et | Et | CH₂ | CH═CH | 4′-MeC(O) | O |
| 93 | MeOC(O) | 2,5-diCl | 3 | n-Pr | n-Pr | CH₂ | CH≡C | 4′-PhC(O) | O |
| 94 | MeOC(O) | 2,5-diCl | 3 | Me | c-Pr | CH₂ | 2-Pyridyl | 4′-MeOC(O) | O |
| 95 | MeOC(O) | 2,5-diCl | 3 | Me | CF₃ | CH₂ | H | — | NH |
| 96 | c-Pentyloxy | 2-Cl | 3 | Me | CH₂═CHCH₂— | CH(Me) | MeSO₂ | — | O |
| 97 | c-Pentyloxy | — | 3 | Me | CH≡CCH₂— | C(Me)₂ | PhSO₂ | — | O |
| 98 | c-Pentyloxy | — | 3 | Me | Ph | NH | N(Me)₂SO₂ | — | O |
| 99 | c-Pentyloxy | — | 3 | Me | 2-Thienyl | N(Me) | H | — | O |
| 100 | c-Pentyloxy | — | 3 | Me | 2-Furyl | CH₂ | CF₃ | 5′-Me | N(Me) |

TABLE 4

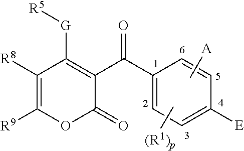

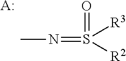

Exanple of Compound (3)

| | | | Position | | | | | | |
|---|---|---|---|---|---|---|---|---|---|
| No. | E | (R¹)ₚ | of a | R² | R³ | R⁵ | R⁸ | R⁹ | G |
| 1 | H | 2-Cl | 3 | Me | Me | H | H | H | O |
| 2 | H | 2-Cl | 3 | Et | Et | CF₃ | H | H | O |
| 3 | H | 2-Cl | 3 | n-Pr | n-Pr | H | H | H | O |
| 4 | H | 2-Cl | 3 | Me | c-Pr | Me | H | H | O |
| 5 | H | 2-Cl | 3 | Me | CF₃ | Ph | H | H | S |
| 6 | Me | 2-Cl | 3 | Me | CH₂═CHCH₂— | PhCH₂ | H | H | O |
| 7 | Me | 2-Cl | 3 | Me | CH≡CCH₂— | Tosyl | H | H | O |
| 8 | Me | 2-Cl | 3 | Me | Ph | MeC(O) | H | H | O |
| 9 | Me | 2-Cl | 3 | Me | 2-Thienyl | PhC(O) | H | H | O |
| 10 | Me | 2-Cl | 3 | Me | 2-Furyl | CH₂═CH | H | H | S(O) |
| 11 | CF₃ | 2-Cl | 3 | Me | 2-Pyridyl | CH≡C | H | Me | O |
| 12 | CF₃ | 2-Cl | 3 | Me | 4-Piperidyl | 2-Pyridyl | H | CF₃ | O |
| 13 | CF₃ | 2-Cl | 3 | —CH₂CH₂CH₂— | | CH₂ | H | i-Pr | O |
| 14 | CF₃ | 2-Cl | 3 | —CH₂CH(Me)CH₂— | | CH₂ | Me | H | O |
| 15 | CF₃ | 2-Cl | 3 | —CH₂CH₂CH₂CH₂— | | PhSO₂ | CF₃ | H | S(O)₂ |
| 16 | MeO | 2-Cl | 3 | —CH₂CH₂CH₂CH₂CH₂— | | N(Me)₂SO₂ | i-Pr | H | O |
| 17 | MeO | 2-Cl | 3 | —CH₂CH₂—O—CH₂CH₂— | | H | Me | Me | O |
| 18 | MeO | 2-Cl | 3 | —CH₂CH₂—C(O)—CH₂CH₂— | | CF₃ | CF₃ | CF₃ | O |
| 19 | MeO | 2-Cl | 3 | —CH₂CH₂CH(OEt)CH₂CH₂— | | H | Me | Et | O |
| 20 | MeO | 2-Cl | 3 | —CH₂CH₂—N(Me)—CH₂CH₂— | | Me | Et | Me | NH |
| 21 | Ph | 2-Cl | 3 | Me | Me | EtOC(O) | H | H | O |
| 22 | Ph | 2-Cl | 3 | Et | Et | MeSC(O) | H | H | O |
| 23 | Ph | 2-Cl | 3 | n-Pr | n-Pr | PhC(O) | H | H | O |
| 24 | Ph | 2-Cl | 3 | Me | c-Pr | Me | H | H | O |
| 25 | Ph | 2-Cl | 3 | Me | CF₃ | PhC(O) | H | H | N(Me) |
| 26 | NH₂ | 2-Cl | 3 | Me | CH₂═CHCH₂— | CH₂═CH | H | H | O |
| 27 | NH₂ | 2-Me | 3 | Me | CH≡CCH₂— | CH≡C | H | H | O |
| 28 | NH₂ | 2-Me | 3 | Me | Ph | 2-Pyridyl | H | H | O |
| 29 | NH₂ | 2-Me | 3 | Me | 2-Thienyl | H | H | H | O |
| 30 | NH₂ | 2-Me | 3 | Me | 2-Furyl | MeSO₂ | H | H | S |
| 31 | N(Me)2 | 2-Cl | 3 | Me | 2-Pyridyl | PhSO₂ | H | Me | O |
| 32 | N(Me)2 | 2-Me | 3 | Me | 4-Piperidyl | N(Me)₂SO₂ | H | CF₃ | O |
| 33 | N(Me)2 | 2-Me | 3 | —CH₂CH₂CH₂— | | H | H | i-Pr | O |
| 34 | N(Me)2 | 2-Me | 3 | —CH₂CH(Me)CH₂— | | CF₃ | Me | H | O |
| 35 | N(Me)2 | 2-Me | 3 | —CH₂CH₂CH₂CH₂— | | H | CF₃ | H | S(O) |
| 36 | HN (Me) C(O) | 2-Cl | 3 | —CH₂CH₂CH₂CH₂CH₂— | | Me | i-Pr | H | O |
| 37 | HN (Me) C(O) | 2-CF₃ | 3 | —CH₂CH₂—O—CH₂CH₂— | | Ph | Me | Me | O |
| 38 | HN (Me) C(O) | 2-CF₃ | 3 | —CH₂CH₂—C(O)—CH₂CH₂— | | PhCH₂ | CF₃ | CF₃ | O |
| 39 | HN (Me) C(O) | 2-CF₃ | 3 | —CH₂CH₂CH(OEt)CH₂CH₂— | | Tosyl | Me | CF₃ | O |
| 40 | HN(Me) C(O) | 2-CF₃ | 3 | —CH₂CH₂—N(Me)—CH₂CH₂— | | MeC(O) | CF₃ | Me | S(O)₂ |
| 41 | MeOC(O) | 2-Cl | 3 | Me | Me | PhC(O) | H | H | O |
| 42 | MeOC(O) | 2-CF₃ | 3 | Et | Et | CH₂═CH | H | H | O |
| 43 | MeOC(O) | 2-CF₃ | 3 | n-Pr | n-Pr | CH≡C | H | H | O |
| 44 | MeOC(O) | 2-CF₃ | 3 | Me | c-Pr | 2-Pyridyl | H | H | O |
| 45 | MeOC(O) | 2-CF₃ | 3 | Me | CF₃ | H | H | H | NH |
| 46 | c-Propoxy | 2-Cl | 3 | Me | CH₂═CHCH₂— | MeSO₂ | H | H | O |
| 47 | c-Pentyloxy | 2-OH | 3 | Me | CH≡CCH₂— | PhSO₂ | H | H | O |
| 48 | c-Pentyloxy | 2-OH | 3 | Me | Ph | N(Me)₂SO₂ | H | H | O |
| 49 | c-Pentyloxy | 2-OH | 3 | Me | 2-Thienyl | H | H | H | O |
| 50 | c-Pentyloxy | 2-OH— | 3 | Me | 2-Furyl | CF₃ | H | Me | N(Me) |
| 51 | H | 2-Cl | 5 | Me | Me | H | H | i-Pr | O |
| 52 | H | 2-MeO | 5 | Et | Et | CF₃ | Me | H | O |
| 53 | H | 2-MeO | 5 | n-Pr | n-Pr | H | CF₃ | H | O |
| 54 | H | 2-MeO | 5 | Me | c-Pr | Me | i-Pr | H | O |
| 55 | H | 2-MeO | 5 | Me | CF₃ | Ph | Me | Me | S |
| 56 | Me | 2-Cl | 5 | Me | CH₂═CHCH₂— | PhCH₂ | CF₃ | CF₃ | O |
| 57 | Me | 2-CF₃O | 5 | Me | CH≡CCH₂— | Tosyl | Me | Et | O |
| 58 | Me | 2-CF₃O | 5 | Me | Ph | MeC(O) | CF₃ | Me | O |
| 59 | Me | 2-CF₃O | 5 | Me | 2-Thienyl | PhC(O) | H | H | O |
| 60 | Me | 2-CF₃O | 5 | Me | 2-Furyl | CH₂═CH | H | H | S(O) |
| 61 | CF₃ | 2-Cl | 5 | Me | 2-Pyridyl | CH≡C | H | H | O |
| 62 | CF₃ | 2-CF₃O | 5 | Me | 4-Piperidyl | 2-Pyridyl | H | H | O |
| 63 | CF₃ | 2-CF₃O | 5 | —CH₂CH₂CH₂— | | H | H | H | O |
| 64 | CF₃ | 2-CF₃O | 5 | —CH₂CH(Me)CH₂— | | MeSO₂ | H | H | O |
| 65 | CF₃ | 2-CF₃O | 5 | —CH₂CH₂CH₂CH₂— | | PhSO₂ | H | H | S(O)₂ |
| 66 | MeO | 2-Cl | 5 | —CH₂CH₂CH₂CH₂CH₂— | | N(Me)₂SO₂ | H | H | O |
| 67 | MeO | 2-SH | 5 | —CH₂CH₂—O—CH₂CH₂— | | H | H | H | O |
| 68 | MeO | 2-SH | 5 | —CH₂CH₂—C(O)—CH₂CH₂— | | CF₃ | H | H | O |
| 69 | MeO | 2-SH | 5 | —CH₂CH₂CH(OEt)CH₂CH₂— | | H | H | H | O |
| 70 | MeO | 2-SH | 5 | —CH₂CH₂—N(Me)—CH₂CH₂— | | Me | H | H | NH |
| 71 | PhC(O) | 2-Cl | 6 | Me | Me | Ph | H | Me | O |
| 72 | Ph | 2-MeS | 6 | Et | Et | PhCH₂ | H | i-Pr | O |
| 73 | Ph | 2-MeS | 6 | n-Pr | n-Pr | Tosyl | Me | H | O |
| 74 | Ph | 2-MeS | 6 | Me | c-Pr | MeC(O) | CF₃ | H | O |
| 75 | Ph | 2-MeS | 6 | Me | CF₃ | PhC(O) | i-Pr | H | N(Me) |
| 76 | NH₂ | 2-Cl | 6 | Me | CH₂═CHCH₂— | CH₂═CH | Me | Me | O |
| 77 | NH₂ | 2-MeS(O)₂ | 6 | Me | CH≡CCH₂— | CH≡C | CF₃ | CF₃ | O |
| 78 | NH₂ | 2-MeS(O)₂ | 6 | Me | Ph | 2-Pyridyl | Me | CF₃ | O |
| 79 | CN | 2-MeS(O)₂ | 6 | Me | 2-Thienyl | H | Et | Me | O |
| 80 | EtS | 2-MeS(O)₂ | 6 | Me | 2-Furyl | MeSO₂ | H | H | S |
| 81 | N(Me)2 | 2-Ph | 6 | Me | 2-Pyridyl | PhSO₂ | H | H | O |
| 82 | N(Me)2 | 2-NO₂ | 6 | Me | 4-Piperidyl | N(Me)₂SO₂ | H | H | O |
| 83 | N(Me)2 | 2-NO₂ | 6 | —CH₂CH₂CH₂— | | H | H | H | O |
| 84 | N(Me)2 | 2-NO₂ | 6 | —CH₂CH(Me)CH₂— | | CF₃ | H | H | O |
| 85 | N(Me)2 | 2-NO₂ | 6 | —CH₂CH₂CH₂CH₂— | | H | H | H | S(O) |
| 86 | HN(Me) C(O) | 2-Cl | 6 | —CH₂CH₂CH₂CH₂CH₂— | | Me | H | H | O |
| 87 | HN (Me) C(O) | 2,5-diMe | 6 | —CH₂CH₂—O—CH₂CH₂— | | Ph | H | H | O |
| 88 | HN (Me) C(O) | 2,5-diMe | 6 | —CH₂CH₂—C(O)—CH₂CH₂— | | PhCH₂ | H | H | O |
| 89 | HN (Me) C(O) | 2,5-diMe | 6 | —CH₂CH₂CH(OEt)CH₂CH₂— | | Tosyl | H | H | O |
| 90 | HN (Me) C(O) | 2,5-diMe | 6 | —CH₂CH₂—N(Me)—CH₂CH₂— | | MeC(O) | H | H | S(O)₂ |
| 91 | MeOC(O) | 2-Cl | 3 | Me | Me | PhC(O) | H | Me | N(Et) |
| 92 | MeOC(O) | 2,5-diCl | 3 | Et | Et | CH₂═CH | H | i-Pr | O |
| 93 | MeOC(O) | 2,5-diCl | 3 | n-Pr | n-Pr | CH≡C | Me | H | O |
| 94 | MeOC(O) | 2,5-diCl | 3 | Me | c-Pr | 2-Pyridyl | Et | H | O |
| 95 | MeOC(O) | 2,5-diMe₂N | 3 | Me | CF₃ | H | i-Pr | H | NH |
| 96 | c-Pentyloxy | 2-Cl | 3 | Me | CH₂═CHCH₂— | MeSO₂ | Me | Me | O |
| 97 | c-Pentyloxy | — | 3 | Me | CH≡CCH₂— | PhSO₂ | Et | Et | O |
| 98 | c-Pentyloxy | — | 3 | Me | Ph | N(Me)₂SO₂ | Me | Et | O |
| 99 | c-Pentyloxy | — | 3 | Me | 2-Thienyl | H | Et | Me | O |
| 100 | 2-Imidazolyl | — | 3 | Me | 2-Furyl | CF₃ | H | H | N(Me) |

TABLE 5

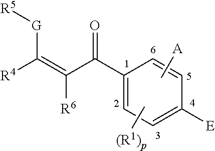

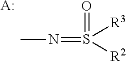

Exanple of Compound (4)

| | | | Position | | | | | | |
|---|---|---|---|---|---|---|---|---|---|
| No. | E | (R¹)p | of A | R² | R³ | R⁵ | R⁴ | R⁶ | G |
| 1 | H | 2-Cl | 3 | Me | Me | H | H | CN | O |
| 2 | H | 2-Cl | 3 | Et | Et | CF₃ | H | CN | O |
| 3 | H | 2-Cl | 3 | n-Pr | n-Pr | H | H | CN | O |
| 4 | H | 2-Cl | 3 | Me | c-Pr | Me | H | CN | O |
| 5 | H | 2-Cl | 3 | Me | CF₃ | Ph | H | CN | S |
| 6 | Me | 2-Cl | 3 | Me | CH₂═CHCH₂— | PhCH₂ | H | CN | O |
| 7 | Me | 2-Cl | 3 | Me | CH≡CCH₂— | Tosyl | H | CN | O |
| 8 | Me | 2-Cl | 3 | Me | Ph | MeC(O) | H | CN | O |
| 9 | Me | 2-Cl | 3 | Me | 2-Thienyl | PhC(O) | H | CN | O |
| 10 | Me | 2-Cl | 3 | Me | 2-Furyl | CH₂═CH | H | CN | S(O) |
| 11 | CF₃ | 2-Cl | 3 | Me | 2-Pyridyl | CH≡C | H | MeC(O) | O |
| 12 | CF₃ | 2-Cl | 3 | Me | 4-Piperidyl | 2-Pyridyl | H | PhC(O) | O |
| 13 | CF₃ | 2-Me₂N | 3 | —CH₂CH₂CH₂— | | H | H | MeOC(O) | O |
| 14 | CF₃ | 2-Cl | 3 | —CH₂CH(Me)CH₂— | | MeSO₂ | H | HN═CPh | O |
| 15 | CF₃ | 2-Cl | 3 | —CH₂CH₂CH₂CH₂— | | PhSO₂ | Me | N(Me)═CH | S(O)₂ |
| 16 | MeO | 2-Cl | 3 | —CH₂CH₂CH₂CH₂CH₂— | | N(Me)₂SO₂ | CF₃ | 1-tetrazolyl | O |
| 17 | MeO | 2-Cl | 3 | —CH₂CH₂—O—CH₂CH₂— | | H | c-Pr | CN | O |
| 18 | MeO | 2-Cl | 3 | —CH₂CH₂—C(O)—CH₂CH₂— | | CF₃ | c-Hexyl | CN | O |
| 19 | MeO | 2-Cl | 3 | —CH₂CH₂CH(OEt)CH₂CH₂— | | H | NH₂ | CN | O |
| 20 | MeO | 2-Cl | 3 | —CH₂CH₂—N(Me)—CH₂CH₂— | | Me | N(Me)₂ | CN | NH |
| 21 | Ph | 2-Cl | 3 | Me | Me | Et | Me | CN | O |
| 22 | Ph | 2-Cl | 3 | Et | Et | 5′-MeC(O) | CF₃ | CN | O |
| 23 | Ph | 2-Cl | 3 | n-Pr | n-Pr | 5′-PhC(O) | c-Pr | CN | O |
| 24 | Ph | 2-Cl | 3 | Me | c-Pr | 5′-MeOC(O) | c-Hexyl | CN | O |
| 25 | Ph | 2-Cl | 3 | Me | CF₃ | PhC(O) | NH₂ | CN | N(Me) |
| 26 | NH₂ | 2-Cl | 3 | Me | CH₂═CHCH₂— | CH₂═CH | N(Me)₂ | CN | O |
| 27 | NH₂ | 2-Me | 3 | Me | CH≡CCH₂— | CH≡C | H | CN | O |
| 28 | NH₂ | 2-Me | 3 | Me | Ph | 2-Pyridyl | H | CN | O |
| 29 | NH₂ | 2-Me | 3 | Me | 2-Thienyl | H | H | MeC(O) | O |
| 30 | NH₂ | 2-Me | 3 | Me | 2-Furyl | MeSO₂ | H | PhC(O) | S |
| 31 | N(Me)2 | 2-Cl | 3 | Me | 2-Pyridyl | PhSO₂ | H | MeOC(O) | O |
| 32 | N(Me)2 | 2-Me | 3 | Me | 4-Piperidyl | N(Me)₂SO₂ | H | HN═CH | O |
| 33 | N(Me)2 | 2-Me | 3 | —CH₂CH₂CH₂— | | MeSC(O) | H | N(Me)═CH | O |
| 34 | N(Me)2 | 2-Me | 3 | —CH₂CH(Me)CH₂— | | CF₃ | H | 1-tetrazolyl | O |
| 35 | N(Me)2 | 2-Me | 3 | —CH₂CH₂CH₂CH₂— | | EtOC(O) | H | CN | S(O) |
| 36 | HN (Me) C(O) | 2-Cl | 3 | —CH₂CH₂CH₂CH₂CH₂— | | Me | H | EtN═CMe | O |
| 37 | HN (Me) C(O) | 2-CF₃ | 3 | —CH₂CH₂—O—CH₂CH₂— | | Ph | H | CN | O |
| 38 | HN (Me) C(O) | 2-CF₃ | 3 | —CH₂CH₂—C(O)—CH₂CH₂— | | PhCH₂ | H | CN | O |
| 39 | HN (Me) C(O) | 2-CF₃ | 3 | —CH₂CH₂CH(OEt)CH₂CH₂— | | Tosyl | H | CN | O |
| 40 | HN(Me) C(O) | 2-CF₃ | 3 | —CH₂CH₂—N(Me)—CH₂CH₂— | | MeC(O) | H | CN | S(O)₂ |
| 41 | MeOC(O) | 2-Cl | 3 | Me | Me | PhC(O) | H | CN | O |
| 42 | MeOC(O) | 2-CF₃ | 3 | Et | Et | CH₂═CH | H | CN | O |
| 43 | MeOC(O) | 2-CF₃ | 3 | n-Pr | n-Pr | CH≡C | H | CN | O |
| 44 | MeOC(O) | 2-CF₃ | 3 | Me | c-Pr | 2-Pyridyl | H | CN | O |
| 45 | MeOC(O) | 2-CF₃ | 3 | Me | CF₃ | H | Me | CN | NH |
| 46 | c-Pentyloxy | 2-Cl | 3 | Me | CH₂═CHCH₂— | MeSO₂ | CF₃ | CN | O |
| 47 | c-Pentyloxy | 2-OH | 3 | Me | CH≡CCH₂— | PhSO₂ | c-Pr | MeC(O) | O |
| 48 | c-Pentyloxy | 2-OH | 3 | Me | Ph | N(Me)₂SO₂ | c-Hexyl | PhC(O) | O |
| 49 | c-Pentyloxy | 2-OH | 3 | Me | 2-Thienyl | H | NH₂ | MeOC(O) | O |
| 50 | c-Pentyloxy | 2-OH | 3 | Me | 2-Furyl | CF₃ | N(Me)₂ | HN═CH | N(Me) |
| 51 | H | 2-Cl | 5 | Me | Me | H | H | N(Me)═CH | O |
| 52 | H | 2-MeO | 5 | Et | Et | CF₃ | H | 5-tetrazolyl | O |
| 53 | H | 2-MeO | 5 | n-Pr | n-Pr | H | H | CN | O |
| 54 | H | 2-MeO | 5 | Me | c-Pr | Me | H | CN | O |
| 55 | PhC(O) | 2-MeO | 5 | Me | CF₃ | Ph | H | CN | S |
| 56 | CN | 2-Cl | 5 | Me | CH₂═CHCH₂— | PhCH₂ | H | CN | O |
| 57 | EtS | 2-CF₃O | 5 | Me | CH≡CCH₂— | Tosyl | H | CN | O |
| 58 | Me | 2-CF₃O | 5 | Me | Ph | MeC(O) | H | CN | O |
| 59 | Me | 2-CF₃O | 5 | Me | 2-Thienyl | PhC(O) | H | CN | O |
| 60 | Me | 2-CF₃O | 5 | Me | 2-Furyl | CH₂═CH | H | CN | S(O) |
| 61 | CF₃ | 2-Cl | 5 | Me | 2-Pyridyl | CH≡C | H | CN | O |
| 62 | CF₃ | 2-CF₃O | 5 | Me | 4-Piperidyl | 2-Pyridyl | H | CN | O |
| 63 | CF₃ | 2-CF₃O | 5 | —CH₂CH₂CH₂— | | H | Me | MeC(O) | O |
| 64 | CF₃ | 2-CF₃O | 5 | —CH₂CH(Me)CH₂— | | MeSO₂ | CF₃ | PhC(O) | O |
| 65 | CF₃ | 2-CF₃O | 5 | —CH₂CH₂CH₂CH₂— | | PhSO₂ | c-Pr | MeOC(O) | S(O)₂ |
| 66 | MeO | 2-Cl | 5 | —CH₂CH₂CH₂CH₂CH₂— | | N(Me)₂SO₂ | c-Hexyl | HN═CH | O |
| 67 | MeO | 2-SH | 5 | —CH₂CH₂—O—CH₂CH₂— | | H | NH₂ | N(Me)═CH | O |
| 68 | MeO | 2-SH | 5 | —CH₂CH₂—C(O)—CH₂CH₂— | | CF₃ | N(Me)₂ | 1-tetrazolyl | O |
| 69 | MeO | 2-SH | 5 | —CH₂CH₂CH(OEt)CH₂CH₂— | | H | H | CN | O |
| 70 | MeO | 2-SH | 5 | —CH₂CH₂—N(Me)—CH₂CH₂— | | Me | H | CN | NH |
| 71 | 3-Pyridyl | 2-Cl | 6 | Me | Me | Ph | H | CN | O |
| 72 | Ph | 2-MeS | 6 | Et | Et | PhCH₂ | H | CN | O |
| 73 | Ph | 2-MeS | 6 | n-Pr | n-Pr | Tosyl | H | CN | O |
| 74 | Ph | 2-MeS | 6 | Me | c-Pr | MeC(O) | H | CN | O |
| 75 | Ph | 2-MeS | 6 | Me | CF₃ | PhC(O) | H | CN | N(Me) |
| 76 | NH₂ | 2-Cl | 6 | Me | CH₂═CHCH₂— | CH₂═CH | H | CN | O |
| 77 | NH₂ | 2-MeS(O)₂ | 6 | Me | CH≡CCH₂— | CH≡C | H | MeC(O) | O |
| 78 | NH₂ | 2-MeS(O)₂ | 6 | Me | Ph | 2-Pyridyl | H | PhC(O) | O |
| 79 | NH₂ | 2-MeS(O)₂ | 6 | Me | 2-Thienyl | H | H | MeOC(O) | O |
| 80 | NH₂ | 2-MeS(O)₂ | 6 | Me | 2-Furyl | Me₂NC(O) | H | HN═CH | S |
| 81 | N(Me)2 | 2-Cl | 6 | Me | 2-Pyridyl | PhSO₂ | H | N(Me)═CH | O |
| 82 | N(Me)2 | 2-NO₂ | 6 | Me | 4-Piperidyl | N(Me)₂SO₂ | H | 5-tetrazolyl | O |
| 83 | N(Me)2 | 2-NO₂ | 6 | —CH₂CH₂CH₂— | | H | H | CN | O |
| 84 | N(Me)2 | 2-NO₂ | 6 | —CH₂CH(Me)CH₂— | | CF₃ | H | CN | O |
| 85 | N(Me)2 | 2-NO₂ | 6 | —CH₂CH₂CH₂CH₂— | | H | H | CN | S(O) |
| 86 | HN(Me) C(O) | 2-Cl | 6 | —CH₂CH₂CH₂CH₂CH₂— | | Me | H | CN | O |
| 87 | HN (Me) C(O) | 2,5-diMe | 6 | —CH₂CH₂—O—CH₂CH₂— | | Ph | H | CN | O |
| 88 | HN (Me) C(O) | 2,5-diMe | 6 | —CH₂CH₂—C(O)—CH₂CH₂— | | PhCH₂ | H | CN | O |
| 89 | HN (Me) C(O) | 2,5-diMe | 6 | —CH₂CH₂CH(OEt)CH₂CH₂— | | Tosyl | H | CN | O |
| 90 | HN (Me) C(O) | 2,5-diMe | 6 | —CH₂CH₂—N(Me)—CH₂CH₂— | | MeC(O) | H | CN | S(O)₂ |
| 91 | MeOC(O) | 2-Cl | 3 | Me | Me | PhC(O) | Me | CN | O |
| 92 | MeOC(O) | 2,5-diCl | 3 | Et | Et | CH₂═CH | CF₃ | CN | O |
| 93 | MeOC(O) | 2,5-diCl | 3 | n-Pr | n-Pr | CH≡C | c-Pr | CN | O |
| 94 | MeOC(O) | 2,5-diCl | 3 | Me | c-Pr | 2-Pyridyl | c-Hexyl | CN | O |
| 95 | MeOC(O) | 2,5-diCl | 3 | Me | CF₃ | H | NH₂ | CN | NH |
| 96 | c-Pentyloxy | 2-Cl | 3 | Me | CH₂═CHCH₂— | MeSO₂ | N(Me)₂ | CN | O |
| 97 | c-Pentyloxy | — | 3 | Me | CH≡CCH₂— | PhSO₂ | H | CN | O |
| 98 | c-Pentyloxy | — | 3 | Me | Ph | N(Me)₂SO₂ | H | CN | O |
| 99 | c-Pentyloxy | — | 3 | Me | 2-Thienyl | H | Et | CN | O |
| 100 | c-Pentyloxy | — | 3 | Me | 2-Furyl | CF₃ | H | CN | N(Me) |

TABLE 6

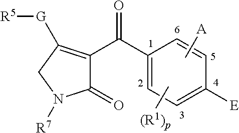

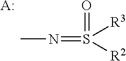

Exanple of Compound (5)

| | | | Position | | | | | |
|---|---|---|---|---|---|---|---|---|
| No. | E | (R¹)p | of A | R² | R³ | R⁵ | R⁷ | G |
| 1 | MeC(O) | 2-Cl | 3 | Me | Me | H | H | O |
| 2 | 2-Thienyl | 2-Cl | 3 | Et | Et | CF₃ | H | O |
| 3 | H | 2-Cl | 3 | n-Pr | n-Pr | H | H | O |
| 4 | H | 2-Cl | 3 | Me | c-Pr | Me | H | O |
| 5 | H | 2-Cl | 3 | Me | CF₃ | Ph | H | S |
| 6 | Me | 2-Cl | 3 | Me | CH₂═CHCH₂— | PhCH₂ | H | O |
| 7 | Me | 2-Cl | 3 | Me | CH≡CCH₂— | Tosyl | H | O |
| 8 | Me | 2-Cl | 3 | Me | Ph | MeC(O) | H | O |
| 9 | Me | 2-Cl | 3 | Me | 2-Thienyl | PhC(O) | H | O |
| 10 | Me | 2-Cl | 3 | Me | 2-Furyl | CH₂═CH | H | S(O) |
| 11 | CF₃ | 2-Cl | 3 | Me | 2-Pyridyl | CH≡C | H | O |
| 12 | CF₃ | 2-Cl | 3 | Me | 4-Piperidyl | 2-Pyridyl | H | O |
| 13 | CF₃ | 2-Cl | 3 | —CH₂CH₂CH₂— | | CH₂ | H | O |
| 14 | CF₃ | 2-Cl | 3 | —CH₂CH(Me)CH₂— | | CH₂ | Me | O |
| 15 | CF₃ | 2-NH₂ | 3 | —CH₂CH₂CH₂CH₂— | | PhSO₂ | CF₃ | S(O)₂ |
| 16 | MeO | 2-Cl | 3 | —CH₂CH₂CH₂CH₂CH₂— | | N(Me)₂SO₂ | i-Pr | O |
| 17 | MeO | 2-Cl | 3 | —CH₂CH₂—O—CH₂CH₂— | | H | MeO | O |
| 18 | MeO | 2-Cl | 3 | —CH₂CH₂—C(O)—CH₂CH₂— | | CF₃ | EtO | O |
| 19 | MeO | 2-Cl | 3 | —CH₂CH₂CH(OEt)CH₂CH₂— | | H | H | O |
| 20 | MeO | 2-Cl | 3 | —CH₂CH₂—N(Me)—CH₂CH₂— | | Me | H | NH |
| 21 | Ph | 2-Cl | 3 | Me | Me | MeOC(O) | Me | O |
| 22 | Ph | 2-Cl | 3 | Et | Et | EtSC(O) | CF₃ | O |
| 23 | Ph | 2-Cl | 3 | n-Pr | n-Pr | Et₂NC(O) | i-Pr | O |
| 24 | Ph | 2-Cl | 3 | Me | c-Pr | Me₂NC(O) | MeO | O |
| 25 | Ph | 2-Cl | 3 | Me | CF₃ | PhC(O) | EtO | N(Me) |
| 26 | NH₂ | 2-Cl | 3 | Me | CH₂═CHCH₂— | CH₂═CH | H | O |
| 27 | NH₂ | 2-Me | 3 | Me | CH≡CCH₂— | CH≡C | H | O |
| 28 | NH₂ | 2-Me | 3 | Me | Ph | 2-Pyridyl | H | O |
| 29 | NH₂ | 2-Me | 3 | Me | 2-Thienyl | H | H | O |
| 30 | NH₂ | 2-Me | 3 | Me | 2-Furyl | MeSO₂ | H | S |
| 31 | N(Me)2 | 2-Cl | 3 | Me | 2-Pyridyl | PhSO₂ | H | O |
| 32 | N(Me)2 | 2-Me | 3 | Me | 4-Piperidyl | N(Me)₂SO₂ | H | O |
| 33 | N(Me)2 | 2-Me | 3 | —CH₂CH₂CH₂— | | H | H | O |
| 34 | N(Me)2 | 2-Me | 3 | —CH₂CH(Me)CH₂— | | CF₃ | H | O |
| 35 | N(Me)2 | 2-Me | 3 | —CH₂CH₂CH₂CH₂— | | H | H | S(O) |
| 36 | HN (Me) C(O) | 2-Cl | 3 | —CH₂CH₂CH₂CH₂CH₂— | | Me | H | O |
| 37 | HN (Me) C(O) | 2-CF₃ | 3 | —CH₂CH₂—O—CH₂CH₂— | | Ph | H | O |
| 38 | HN (Me) C(O) | 2-CF₃ | 3 | —CH₂CH₂—C(O)—CH₂CH₂— | | PhCH₂ | H | O |
| 39 | HN (Me) C(O) | 2-CF₃ | 3 | —CH₂CH₂CH(OEt)CH₂CH₂— | | Tosyl | H | O |
| 40 | HN(Me) C(O) | 2-CF₃ | 3 | —CH₂CH₂—N(Me)—CH₂CH₂— | | MeC(O) | H | S(O)₂ |
| 41 | MeOC(O) | 2-Cl | 3 | Me | Me | PhC(O) | H | O |
| 42 | MeOC(O) | 2-CF₃ | 3 | Et | Et | CH₂═CH | H | O |
| 43 | MeOC(O) | 2-CF₃ | 3 | n-Pr | n-Pr | CH≡C | H | O |
| 44 | MeOC(O) | 2-CF₃ | 3 | Me | c-Pr | 2-Pyridyl | H | O |
| 45 | MeOC(O) | 2-CF₃ | 3 | Me | CF₃ | H | H | NH |
| 46 | c-Pentyloxy | 2-Cl | 3 | Me | CH₂═CHCH₂— | MeSO₂ | H | O |
| 47 | c-Pentyloxy | 2-OH | 3 | Me | CH≡CCH₂— | PhSO₂ | H | O |
| 48 | c-Pentyloxy | 2-OH | 3 | Me | Ph | N(Me)₂SO₂ | H | O |
| 49 | c-Pentyloxy | 2-OH | 3 | Me | 2-Thienyl | H | H | O |
| 50 | c-Pentyloxy | 2-OH | 3 | Me | 2-Furyl | CF₃ | H | N(Me) |
| 51 | H | 2-Cl | 5 | Me | Me | H | H | O |
| 52 | H | 2-MeO | 5 | Et | Et | CF₃ | Me | O |
| 53 | H | 2-MeO | 5 | n-Pr | n-Pr | H | CF₃ | O |
| 54 | H | 2-MeO | 5 | Me | c-Pr | Me | i-Pr | O |
| 55 | H | 2-MeO | 5 | Me | CF₃ | Ph | MeO | S |
| 56 | Me | 2-Cl | 5 | Me | CH₂═CHCH₂— | PhCH₂ | EtO | O |
| 57 | Me | 2-CF₃O | 5 | Me | CH≡CCH₂— | Tosyl | i-Pr O | O |
| 58 | Me | 2-CF₃O | 5 | Me | Ph | MeC(O) | H | O |
| 59 | Me | 2-CF₃O | 5 | Me | 2-Thienyl | PhC(O) | H | O |
| 60 | MeS | 2-CF₃O | 5 | Me | 2-Furyl | CH₂═CH | H | S(O) |
| 61 | CN | 2-Cl | 5 | Me | 2-Pyridyl | CH≡C | H | O |
| 62 | CF₃ | 2-CF₃O | 5 | Me | 4-Piperidyl | 2-Pyridyl | H | O |
| 63 | CF₃ | 2-CF₃O | 5 | —CH₂CH₂CH₂— | | H | H | O |
| 64 | CF₃ | 2-CF₃O | 5 | —CH₂CH(Me)CH₂— | | MeSO₂ | H | O |
| 65 | CF₃ | 2-CF₃O | 5 | —CH₂CH₂CH₂CH₂— | | PhSO₂ | H | S(O)₂ |
| 66 | MeO | 2-Cl | 5 | —CH₂CH₂CH₂CH₂CH₂— | | N(Me)₂SO₂ | H | O |
| 67 | MeO | 2-SH | 5 | —CH₂CH₂—O—CH₂CH₂— | | H | H | O |
| 68 | MeO | 2-SH | 5 | —CH₂CH₂—C(O)—CH₂CH₂— | | CF₃ | H | O |
| 69 | MeO | 2-SH | 5 | —CH₂CH₂CH(OEt)CH₂CH₂— | | H | H | O |
| 70 | MeO | 2-SH | 5 | —CH₂CH₂—N(Me)—CH₂CH₂— | | Me | H | NH |
| 71 | Ph | 2-Cl | 6 | Me | Me | Ph | H | O |
| 72 | Ph | 2-MeS | 6 | Et | Et | PhCH₂ | H | O |
| 73 | Ph | 2-MeS | 6 | n-Pr | n-Pr | Tosyl | Me | O |
| 74 | Ph | 2-MeS | 6 | Me | c-Pr | MeC(O) | CF₃ | O |
| 75 | Ph | 2-MeS | 6 | Me | CF₃ | PhC(O) | i-Pr | N(Me) |
| 76 | NH₂ | 2-Cl | 6 | Me | CH₂═CHCH₂— | CH₂═CH | MeO | O |
| 77 | NH₂ | 2-MeS(O)₂ | 6 | Me | CH≡CCH₂— | CH≡C | EtO | O |
| 78 | NH₂ | 2-MeS(O)₂ | 6 | Me | Ph | 2-Pyridyl | i-PrO | O |
| 79 | NH₂ | 2-MeS(O)₂ | 6 | Me | 2-Thienyl | H | H | O |
| 80 | NH₂ | 2-MeS(O)₂ | 6 | Me | 2-Furyl | MeSO₂ | H | S |
| 81 | N(Me)2 | 2-Cl | 6 | Me | 2-Pyridyl | PhSO₂ | H | O |
| 82 | N(Me)2 | 2-NO₂ | 6 | Me | 4-Piperidyl | N(Me)₂SO₂ | H | O |
| 83 | N(Me)2 | 2-NO₂ | 6 | —CH₂CH₂CH₂— | | H | H | O |
| 84 | N(Me)2 | 2-NO₂ | 6 | —CH₂CH(Me)CH₂— | | CF₃ | H | O |
| 85 | N(Me)2 | 2-NO₂ | 6 | —CH₂CH₂CH₂CH₂— | | H | H | S(O) |
| 86 | HN(Me) C(O) | 2-Cl | 6 | —CH₂CH₂CH₂CH₂CH₂— | | Me | H | O |
| 87 | HN (Me) C(O) | 2,5-diMe | 6 | —CH₂CH₂—O—CH₂CH₂— | | Ph | H | O |
| 88 | HN (Me) C(O) | 2,5-diMe | 6 | —CH₂CH₂—C(O)—CH₂CH₂— | | PhCH₂ | H | O |
| 89 | HN (Me) C(O) | 2,5-diMe | 6 | —CH₂CH₂CH(OEt)CH₂CH₂— | | Tosyl | H | O |
| 90 | HN (Me) C(O) | 2,5-diMe | 6 | —CH₂CH₂—N(Me)—CH₂CH₂— | | MeC(O) | H | S(O)₂ |
| 91 | MeOC(O) | 2-Cl | 3 | Me | Me | PhC(O) | H | S |
| 92 | MeOC(O) | 2,5-diCl | 3 | Et | Et | CH₂═CH | H | O |
| 93 | MeOC(O) | 2,5-diCl | 3 | n-Pr | n-Pr | CH≡C | Me | O |
| 94 | MeOC(O) | 2,5-diCl | 3 | Me | c-Pr | 2-Pyridyl | CF₃ | O |
| 95 | MeOC(O) | 2,5-diCl | 3 | Me | CF₃ | H | i-Pr | NH |
| 96 | c-Pentyloxy | 2-Cl | 3 | Me | CH₂═CHCH₂— | MeSO₂ | MeO | O |
| 97 | c-Pentyloxy | — | 3 | Me | CH≡CCH₂— | PhSO₂ | EtO | O |
| 98 | c-Pentyloxy | — | 3 | Me | Ph | N(Me)₂SO₂ | i-PrO | O |
| 99 | c-Pentyloxy | — | 3 | Me | 2-Thienyl | H | H | O |
| 100 | c-Pentyloxy | — | 3 | Me | 2-Furyl | CF₃ | H | N(Me) |

TABLE 7

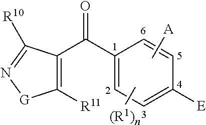

Example of Compound (6)

| | | | Position | | | | | |
|---|---|---|---|---|---|---|---|---|
| No. | E | (R¹)ₚ | of A | R² | R³ | R¹⁰ | R¹¹ | G |
| 1 | H | 2-Cl | 3 | Me | Me | H | H | O |
| 2 | H | 2-Cl | 3 | Et | Et | H | H | O |
| 3 | H | 2-Cl | 3 | n-Pr | n-Pr | H | H | O |
| 4 | 2-Furyl | 2-Cl | 3 | Me | c-Pr | H | H | O |
| 5 | PhC(O) | 2-Cl | 3 | Me | CF₃ | H | H | S |
| 6 | Me | 2-Cl | 3 | Me | CH₂═CHCH₂— | H | H | O |
| 7 | Me | 2-Cl | 3 | Me | CH≡CCH₂— | H | H | O |
| 8 | Me | 2-Cl | 3 | Me | Ph | H | H | O |
| 9 | Me | 2-Cl | 3 | Me | 2-Thienyl | H | H | O |
| 10 | Me | 2-Cl | 3 | Me | 2-Furyl | H | H | S(O) |
| 11 | CF₃ | 2-Cl | 3 | Me | 2-Pyridyl | Me | H | O |
| 12 | CF₃ | 2-Cl | 3 | Me | 4-Piperidyl | CF₃ | H | O |
| 13 | CF₃ | 2-Cl | 3 | —CH₂CH₂CH₂— | | i-Pr | H | O |
| 14 | CF₃ | 2-Cl | 3 | —CH₂CH(Me)CH₂— | | c-Pr | Me | O |
| 15 | CF₃ | 2-Cl | 3 | —CH₂CH₂CH₂CH₂— | | c-Hexyl | CF₃ | S(O)₂ |
| 16 | MeO | 2-Cl | 3 | —CH₂CH₂CH₂CH₂CH₂— | | H | c-Pr | O |
| 17 | MeO | 2-Cl | 3 | —CH₂CH₂—O—CH₂CH₂— | | H | c-Hexyl | O |
| 18 | MeO | 2-Cl | 3 | —CH₂CH₂—C(O)—CH₂CH₂— | | H | H | O |
| 19 | MeO | 2-Cl | 3 | —CH₂CH₂CH(OEt)CH₂CH₂— | | H | H | O |
| 20 | MeO | 2-Cl | 3 | —CH₂CH₂—N(Me)—CH₂CH₂— | | H | H | NH |
| 21 | Ph | 2-Cl | 3 | Me | Me | H | Me | O |
| 22 | Ph | 2-Cl | 3 | Et | Et | H | CF₃ | O |
| 23 | Ph | 2-Cl | 3 | n-Pr | n -Pr | H | i-Pr | O |
| 24 | Ph | 2-Cl | 3 | Me | c-Pr | H | c-Pr | O |
| 25 | Ph | 2-Cl | 3 | Me | CF₃ | Me | c-Hexyl | N(Me) |
| 26 | NH₂ | 2-Cl | 3 | Me | CH₂═CHCH₂— | CF₃ | H | O |
| 27 | NH₂ | 2-Me | 3 | Me | CH≡CCH₂— | i-Pr | H | O |
| 28 | NH₂ | 2-Me | 3 | Me | Ph | c-Pr | H | O |
| 29 | NH₂ | 2-Me | 3 | Me | 2-Thienyl | c-Hexyl | H | O |
| 30 | NH₂ | 2-Me | 3 | Me | 2-Furyl | H | H | S |
| 31 | N(Me)2 | 2-Cl | 3 | Me | 2-Pyridyl | H | H | O |
| 32 | N(Me)2 | 2-Me | 3 | Me | 4-Piperidyl | H | H | O |
| 33 | N(Me)2 | 2-Me | 3 | —CH₂CH₂CH₂— | | H | H | O |
| 34 | N(Me)2 | 2-Me | 3 | —CH₂CH(Me)CH₂— | | H | H | O |
| 35 | N(Me)2 | 2-Me | 3 | —CH₂CH₂CH₂CH₂— | | H | H | S(O) |
| 36 | HN(Me)C(O) | 2-Cl | 3 | —CH₂CH₂CH₂CH₂CH₂— | | Me | H | O |
| 37 | HN(Me)C(O) | 2-CF₃ | 3 | —CH₂CH₂—O—CH₂CH₂— | | CF₃ | H | O |
| 38 | HN(Me)C(O) | 2-CF₃ | 3 | —CH₂CH₂—C(O)—CH₂CH₂— | | i-Pr | H | O |
| 39 | HN(Me)C(O) | 2-CF₃ | 3 | —CH₂CH₂CH(OEt)CH₂CH₂— | | c-Pr | H | O |
| 40 | HN(Me)C(O) | 2-CF₃ | 3 | —CH₂CH₂—N(Me)—CH₂CH₂— | | c-Hexyl | H | S(O)₂ |
| 41 | MeOC(O) | 2-Cl | 3 | Me | Me | H | Me | O |
| 42 | MeOC(O) | 2-CF₃ | 3 | Et | Et | H | CF₃ | O |
| 43 | MeOC(O) | 2-CF₃ | 3 | n-Pr | n-Pr | H | i-Pr | O |
| 44 | MeOC(O) | 2-CF₃ | 3 | Me | c-Pr | H | c-Pr | O |
| 45 | MeOC(O) | 2-CF₃ | 3 | Me | CF₃ | H | c-Hexyl | NH |
| 46 | c-Pentyloxy | 2-Cl | 3 | Me | CH₂═CHCH₂— | H | H | O |
| 47 | c-Pentyloxy | 2-OH | 3 | Me | CH≡CCH₂— | H | H | O |
| 48 | c-Pentyloxy | 2-OH | 3 | Me | Ph | H | H | O |
| 49 | c-Pentyloxy | 2-OH | 3 | Me | 2-Thienyl | H | H | O |
| 50 | c-Pentyloxy | 2-OH - | 3 | Me | 2-Furyl | CF₃ | H | N(Me) |
| 51 | H | 2-Cl | 5 | Me | Me | H | H | O |
| 52 | H | 2-MeO | 5 | Et | Et | CF₃ | Me | O |
| 53 | H | 2-MeO | 5 | n-Pr | n-Pr | H | Et | O |
| 54 | H | 2-MeO | 5 | Me | c-Pr | Me | i-Pr | O |
| 55 | H | 2-MeO | 5 | Me | CF₃ | H | c-Pr | S |
| 56 | Me | 2-Cl | 5 | Me | CH₂═CHCH₂— | H | c-Hexyl | O |
| 57 | Me | 2-CF₃O | 5 | Me | CH≡CCH₂— | H | H | O |
| 58 | Me | 2-CF₃O | 5 | Me | Ph | H | H | O |
| 59 | MeS | 2-CF₃O | 5 | Me | 2-Thienyl | H | H | O |
| 60 | CN | 2-CF₃O | 5 | Me | 2-Furyl | H | H | S(O) |
| 61 | CF₃ | 3-Me₂N | 5 | Me | 2-Pyridyl | H | H | O |
| 62 | CF₃ | 2-CF₃O | 5 | Me | 4-Piperidyl | H | H | O |
| 63 | CF₃ | 2-CF₃O | 5 | —CH₂CH₂CH₂— | | H | H | O |
| 64 | CF₃ | 2-CF₃O | 5 | —CH₂CH(Me)CH₂— | | H | c-Pr | O |
| 65 | CF₃ | 2-CF₃O | 5 | —CH₂CH₂CH₂CH₂— | | H | c-Hexyl | S(O)₂ |
| 66 | MeO | 2-Cl | 5 | —CH₂CH₂CH₂CH₂CH₂— | | H | H | O |
| 67 | MeO | 2-SH | 5 | —CH₂CH₂—O—CH₂CH₂— | | H | H | O |
| 68 | MeO | 2-SH | 5 | —CH₂CH₂—C(O)—CH₂CH₂— | | CF₃ | H | O |
| 69 | MeO | 2-SH | 5 | —CH₂CH₂CH(OEt)CH₂CH₂— | | H | H | O |
| 70 | MeO | 2-SH | 5 | —CH₂CH₂—N(Me)—CH₂CH₂— | | Me | H | NH |
| 71 | Ph | 2-Cl | 6 | Me | Me | CF₃ | H | O |
| 72 | Ph | 2-MeS | 6 | Et | Et | H | H | O |
| 73 | Ph | 2-MeS | 6 | n-Pr | n-Pr | H | Me | O |
| 44 | Ph | 2-MeS | 6 | Me | c-Pr | H | Et | O |
| 75 | Ph | 2-MeS | 6 | Me | CF₃ | H | i-Pr | N(Me) |
| 76 | NH₂ | 2-Cl | 6 | Me | CH₂═CHCH₂— | H | Et | O |
| 77 | NH₂ | 2-MeS(O)₂ | 6 | Me | CH≡CCH₂— | H | c-Pr | O |
| 78 | NH₂ | 2-MeS(O)₂ | 6 | Me | Ph | H | c-Hexyl | O |
| 79 | NH₂ | 2-MeS(O)₂ | 6 | Me | 2-Thienyl | H | H | O |
| 80 | NH₂ | 2-MeS(O)₂ | 6 | Me | 2-Furyl | H | H | S |
| 81 | N(Me)2 | 2-Cl | 6 | Me | 2-Pyridyl | H | H | O |
| 82 | N(Me)2 | 2-NO₂ | 6 | Me | 4-Piperidyl | H | H | O |
| 83 | N(Me)2 | 2-NO₂ | 6 | —CH₂CH₂CH₂— | | H | H | O |
| 84 | N(Me)2 | 2-NO₂ | 6 | —CH₂CH(Me)CH₂— | | CF₃ | H | O |
| 85 | N(Me)2 | 2-NO₂ | 6 | —CH₂CH₂CH₂CH₂— | | H | H | S(O) |
| 86 | HN(Me)C(O) | 2-Cl | 6 | —CH₂CH₂CH₂CH₂CH₂— | | Me | H | O |
| 87 | HN(Me)C(O) | 2,5-diMe | 6 | —CH₂CH₂—O—CH₂CH₂— | | H | H | O |
| 88 | HN(Me)C(O) | 2,5-diMe | 6 | —CH₂CH₂—C(O)—CH₂CH₂— | | H | H | O |
| 89 | HN(Me)C(O) | 2,5-diMe | 6 | —CH₂CH₂CH(OEt)CH₂CH₂— | | H | H | O |
| 90 | HN(Me)C(O) | 2,5-diMe | 6 | —CH₂CH₂—N(Me)—CH₂CH₂— | | H | H | S(O)₂ |
| 91 | MeOC(O) | 2-Cl | 3 | Me | Me | H | H | O |
| 92 | MeOC(O) | 2,5-diCl | 3 | Et | Et | H | H | O |
| 93 | MeOC(O) | 2,5-diCl | 3 | n-Pr | n-Pr | Me | Me | O |
| 94 | MeOC(O) | 2,5-diCl | 3 | Me | c-Pr | c-Pr | Et | O |
| 95 | MeOC(O) | 2,5-diCl | 3 | Me | CF₃ | c-Hexyl | i-Pr | NH |
| 96 | c-Pentyloxy | 2-Cl | 3 | Me | CH₂═CHCH₂— | H | c-Pr | O |
| 97 | c-Pentyloxy | — | 3 | Me | CH≡CCH₂— | H | c-Hexyl | O |
| 98 | c-Pentyloxy | — | 3 | Me | Ph | H | H | O |
| 99 | c-Pentyloxy | — | 3 | Me | 2-Thienyl | H | H | O |
| 100 | c-Pentyloxy | — | 3 | Me | 2-Furyl | CF₃ | H | N(Me) |

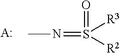

TABLE 8

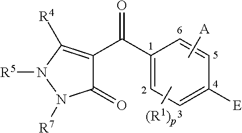

Example of Compound (7)

| | | | Position | | | | | |
|---|---|---|---|---|---|---|---|---|
| No. | E | (R¹)ₚ | of A | R² | R³ | R⁵ | R⁷ | R⁴ |
| 1 | H | 2-Cl | 3 | Me | Me | H | H | H |
| 2 | H | 2-Cl | 3 | Et | Et | CF₃ | H | H |
| 3 | H | 2-Cl | 3 | n-Pr | n-Pr | H | H | H |
| 4 | H | 2-Cl | 3 | Me | c-Pr | Me | H | H |
| 5 | H | 2-Cl | 3 | Me | CF₃ | Ph | H | Me |
| 6 | Me | 2-Cl | 3 | Me | CH₂═CHCH₂— | PhCH₂ | H | H |
| 7 | Me | 2-Cl | 3 | Me | CH≡CCH₂— | Tosyl | H | H |
| 8 | Me | 2-Cl | 3 | Me | Ph | MeC(O) | H | H |
| 9 | Me | 2-Cl | 3 | Me | 2-Thienyl | PhC(O) | H | H |
| 10 | Me | 2-Cl | 3 | Me | 2-Furyl | CH₂═CH | H | CF₃ |
| 11 | CF₃ | 2-Cl | 3 | Me | 2-Pyridyl | CH≡C | H | H |
| 12 | CF₃ | 2-Cl | 3 | Me | 4-Piperidyl | 2-Pyridyl | H | H |
| 13 | CF₃ | 2-Cl | 3 | —CH₂CH₂CH₂— | | CH₂ | H | H |
| 14 | CF₃ | 2-Cl | 3 | —CH₂CH(Me)—CH₂— | | CH₂ | Me | H |
| 15 | CF₃ | 2-NH₂ | 3 | —CH₂CH₂CH₂CH₂— | | PhSO₂ | CF₃ | c-Pr |
| 16 | MeO | 2-Cl | 3 | —CH₂CH₂CH₂CH₂CH₂— | | N(Me)₂SO₂ | i-Pr | H |
| 17 | MeO | 2-Cl | 3 | —CH₂CH₂—O—CH₂CH₂— | | H | Me O | H |
| 18 | MeO | 2-Cl | 3 | —CH₂CH₂—C(O)—CH₂CH₂— | | CF₃ | EtO | H |
| 19 | MeO | 2-Cl | 3 | —CH₂CH₂CH(OEt)CH₂CH₂— | | H | H | H |
| 20 | MeO | 2-Cl | 3 | —CH₂CH₂—N(Me)—CH₂CH₂— | | Me | H | NH₂ |
| 21 | Ph | 2-Cl | 3 | Me | Me | MeOC(O) | Me | H |
| 22 | Ph | 2-Cl | 3 | Et | Et | MeSC(O) | CF₃ | H |
| 23 | Ph | 2-Cl | 3 | n-Pr | n-Pr | PhC(O) | i-Pr | H |
| 24 | Ph | 2-Cl | 3 | Me | c-Pr | H₂NC(O) | MeO | H |
| 25 | Ph | 2-Cl | 3 | Me | CF₃ | PhC(O) | EtO | N(Me) |
| 26 | NH₂ | 2-Cl | 3 | Me | CH₂═CHCH₂— | CH₂═CH | H | H |
| 27 | NH₂ | 2-Me | 3 | Me | CH≡CCH₂— | CH≡C | H | H |
| 28 | NH₂ | 2-Me | 3 | Me | Ph | 2-Pyridyl | H | H |
| 29 | NH₂ | 2-Me | 3 | Me | 2-Thienyl | H | H | H |
| 30 | NH₂ | 2-Me | 3 | Me | 2-Furyl | MeSO₂ | H | Me |
| 31 | N(Me)2 | 2-Cl | 3 | Me | 2-Pyridyl | PhSO₂ | H | H |
| 32 | N(Me)2 | 2-Me | 3 | Me | 4-Piperidyl | N(Me)₂SO₂ | H | H |
| 33 | N(Me)2 | 2-Me | 3 | —CH₂CH₂CH₂— | | H | H | H |
| 34 | N(Me)2 | 2-Me | 3 | —CH₂CH(Me)CH₂— | | CF₃ | H | H |
| 35 | N(Me)2 | 2-Me | 3 | —CH₂CH₂CH₂CH₂— | | H | H | CF₃ |
| 36 | HN(Me)C(O) | 2-Cl | 3 | —CH₂CH₂CH₂CH₂CH₂— | | Me | H | H |
| 37 | HN(Me)C(O) | 2-CF₃ | 3 | —CH₂CH₂—O—CH₂CH₂— | | Ph | H | H |
| 38 | HN(Me)C(O) | 2-CF₃ | 3 | —CH₂CH₂—C(O)—CH₂CH₂— | | PhCH₂ | H | H |
| 39 | HN(Me)C(O) | 2-CF₃ | 3 | —CH₂CH₂CH(OEt)CH₂CH₂— | | Tosyl | H | H |
| 40 | HN(Me)C(O) | 2-CF₃ | 3 | —CH₂CH₂—N(Me)—CH₂CH₂— | | MeC(O) | H | c-Pr |
| 41 | MeOC(O) | 2-Cl | 3 | Me | Me | PhC(O) | H | H |
| 42 | MeOC(O) | 2-CF₃ | 3 | Et | Et | CH₂═CH | H | H |
| 43 | MeOC(O) | 2-CF₃ | 3 | n-Pr | n-Pr | CH≡C | H | H |
| 44 | MeOC(O) | 2-CF₃ | 3 | Me | c-Pr | 2-Pyridyl | H | H |
| 45 | MeOC(O) | 2-CF₃ | 3 | Me | CF₃ | H | H | NH₂ |
| 46 | c-Pentyloxy | 2-Cl | 3 | Me | CH₂═CHCH₂— | MeSO₂ | H | H |
| 47 | c-Pentyloxy | 2-OH | 3 | Me | CH≡CCH₂— | PhSO₂ | H | H |
| 48 | c-Pentyloxy | 2-OH | 3 | Me | Ph | N(Me)₂SO₂ | H | H |
| 49 | c-Pentyloxy | 2-OH | 3 | Me | 2-Thienyl | H | H | H |
| 50 | c-Pentyloxy | 2-OH - | 3 | Me | 2-Furyl | CF₃ | H | N(Me) |
| 51 | MeS | 2-Cl | 5 | Me | Me | H | H | H |
| 52 | CN | 2-MeO | 5 | Et | Et | CF₃ | Me | H |
| 53 | EtC(O) | 2-MeO | 5 | n-Pr | n-Pr | H | CF₃ | H |
| 54 | 2-Pyridyl | 2-MeO | 5 | Me | c-Pr | Me | i-Pr | H |
| 55 | H | 2-MeO | 5 | Me | CF₃ | Ph | MeO | Me |
| 56 | Me | 2-Cl | 5 | Me | CH₂═CHCH₂— | PhCH₂ | EtO | H |
| 57 | Me | 2-CF₃O | 5 | Me | CH≡CCH₂— | Tosyl | i-PrO | H |
| 58 | Me | 2-CF₃O | 5 | Me | Ph | MeC(O) | H | H |
| 59 | Me | 2-CF₃O | 5 | Me | 2-Thienyl | PhC(O) | H | H |
| 60 | Me | 2-CF₃O | 5 | Me | 2-Furyl | CH₂═CH | H | CF₃ |
| 61 | CF₃ | 2-Cl | 5 | Me | 2-Pyridyl | CH≡C | H | H |
| 62 | CF₃ | 2-CF₃O | 5 | Me | 4-Piperidyl | 2-Pyridyl | H | H |
| 63 | CF₃ | 2-CF₃O | 5 | —CH₂CH₂CH₂— | | H | H | H |
| 64 | CF₃ | 2-CF₃O | 5 | —CH₂CH(Me)CH₂— | | MeSO₂ | H | H |
| 65 | CF₃ | 2-CF₃O | 5 | —CH₂CH₂CH₂CH₂— | | PhSO₂ | H | c-Pr |
| 66 | MeO | 2-Cl | 5 | —CH₂CH₂CH₂CH₂CH₂— | | N(Me)₂SO₂ | H | H |
| 67 | MeO | 2-SH | 5 | —CH₂CH₂—O—CH₂CH₂— | | H | H | H |
| 68 | MeO | 2-SH | 5 | —CH₂CH₂—C(O)—CH₂CH₂— | | CF₃ | H | H |
| 69 | MeO | 2-SH | 5 | —CH₂CH₂CH(OEt)CH₂CH₂— | | H | H | H |
| 70 | MeO | 2-SH | 5 | —CH₂CH₂—N(Me)—CH₂CH₂— | | Me | H | NH₂ |
| 71 | Ph | 2-Cl | 6 | Me | Me | Ph | H | H |
| 72 | Ph | 2-MeS | 6 | Et | Et | PhCH₂ | H | H |
| 73 | Ph | 2-MeS | 6 | n-Pr | n-Pr | Tosyl | Me | H |
| 44 | Ph | 2-MeS | 6 | Me | c-Pr | MeC(O) | CF₃ | H |
| 75 | Ph | 2-MeS | 6 | Me | CF₃ | PhC(O) | i-Pr | N(Me)₂ |
| 76 | NH₂ | 2-Cl | 6 | Me | CH₂═CHCH₂— | CH₂═CH | MeO | H |
| 77 | NH₂ | 2-MeS(O)₂ | 6 | Me | CH≡CCH₂— | CH ≡C | EtO | H |
| 78 | NH₂ | 2-MeS(O)₂ | 6 | Me | Ph | 2-Pyridyl | i-PrO | H |
| 79 | NH₂ | 2-MeS(O)₂ | 6 | Me | 2-Thienyl | H | H | H |
| 80 | NH₂ | 2-MeS(O)₂ | 6 | Me | 2-Furyl | MeSO₂ | H | Me |
| 81 | N(Me)₂ | 2-Cl | 6 | Me | 2-Pyridyl | PhSO₂ | H | H |
| 82 | N(Me)₂ | 2-NO₂ | 6 | Me | 4-Piperidyl | N(Me)₂SO₂ | H | H |
| 83 | N(Me)₂ | 2-NO₂ | 6 | —CH₂CH₂CH₂— | | H | H | H |
| 84 | N(Me)₂ | 2-NO₂ | 6 | —CH₂CH(Me)CH₂— | | CF₃ | H | H |
| 85 | N(Me)₂ | 2-NO₂ | 6 | —CH₂CH₂CH₂CH₂— | | H | H | CF₃ |
| 86 | HN(Me)C(O) | 2-Cl | 6 | —CH₂CH₂CH₂CH₂CH₂— | | Me | H | H |
| 87 | HN(Me)C(O) | 2,5-diMe | 6 | —CH₂CH₂—O—CH₂CH₂— | | Ph | H | H |
| 88 | HN(Me)C(O) | 2,5-diMe | 6 | —CH₂CH₂—C(O)—CH₂CH₂— | | PhCH₂ | H | H |
| 89 | HN(Me)C(O) | 2,5-diMe | 6 | —CH₂CH₂CH(OEt)CH₂CH₂— | | Tosyl | H | H |
| 90 | HN(Me)C(O) | 2,5-diMe | 6 | —CH₂CH₂—N(Me)—CH₂CH₂— | | MeC(O) | H | c-Pr |
| 91 | MeOC(O) | 2-Cl | 3 | Me | Me | PhC(O) | H | H |
| 92 | MeOC(O) | 2,5-diCl | 3 | Et | Et | CH₂═CH | H | H |
| 93 | MeOC(O) | 2,5-diCl | 3 | n-Pr | n-Pr | CH≡C | Me | H |
| 94 | MeOC(O) | 2,5-diCl | 3 | Me | c-Pr | 2-Pyridyl | CF₃ | H |
| 95 | MeOC(O) | 2,5-diCl | 3 | Me | CF₃ | H | i-Pr | NH₂ |
| 96 | c-Pentyloxy | 2-Cl | 3 | Me | CH₂═CHCH₂— | MeSO₂ | MeO | H |
| 97 | c-Pentyloxy | — | 3 | Me | CH≡CCH₂— | PhSO₂ | EtO | H |
| 98 | c-Pentyloxy | — | 3 | Me | Ph | N(Me)₂SO₂ | i-PrO | H |
| 99 | c-Pentyloxy | — | 3 | Me | 2-Thienyl | H | H | H |
| 100 | c-Pentyloxy | — | 3 | Me | 2-Furyl | CF₃ | H | N(Me)₂ |

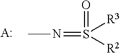

TABLE 9

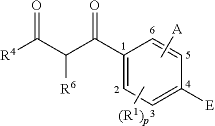

Example of Compound (8)

| | | | Position | | | | |
|---|---|---|---|---|---|---|---|
| No. | E | (R¹)ₚ | of A | R² | R³ | R⁴ | R⁶ |
| 1 | H | 2-Cl | 3 | Me | Me | H | CN |
| 2 | H | 2-Cl | 3 | Et | Et | H | MeC(O) |
| 3 | H | 2-Cl | 3 | n-Pr | n-Pr | H | PhC(O) |
| 4 | H | 2-Cl | 3 | Me | c-Pr | H | MeOC(O) |
| 5 | H | 2-Cl | 3 | Me | CF₃ | H | HN═CH |
| 6 | Me | 2-Cl | 3 | Me | CH₂═CHCH₂— | H | N(Me)═CH |
| 7 | Me | 2-Cl | 3 | Me | CH≡CCH₂— | H | 1-tetrazolyl |
| 8 | Me | 2-Cl | 3 | Me | Ph | H | HC(O) |
| 9 | Me | 2-Cl | 3 | Me | 2-Thenyl | H | EtC(O) |
| 10 | Me | 2-Cl | 3 | Me | 2-Thienyl | H | EtC(O) |
| 11 | CF₃ | 2-Cl | 3 | Me | 2-Pyridyl | H | CN |
| 12 | CF₃ | 2-Cl | 3 | Me | 4-Piperidyl | H | MeC(O) |
| 13 | CF₃ | 2-Cl | 3 | —CH₂CH₂CH₂— | | H | PhC(O) |
| 14 | CF₃ | 2-Cl | 3 | —CH₂CH(Me)CH₂— | | H | MeOC(O) |
| 15 | CF₃ | 2-Cl | 3 | —CH₂CH₂CH₂CH₂— | | Me | HN═CH |
| 16 | MeO | 2-NH₂ | 3 | —CH₂CH₂CH₂CH₂CH₂— | | CF₃ | N(Me)═CH |
| 17 | MeO | 2-Cl | 3 | —CH₂CH₂—O—CH₂CH₂— | | c-Pr | 5-tetrazolyl |
| 18 | MeO | 2-Cl | 3 | —CH₂CH₂—C(O)—CH₂CH₂— | | c-Hexyl | HC(O) |
| 19 | SMO | 2-Cl | 3 | —CH₂CH₂CH(OEt)CH₂CH₂— | | NH₂ | EtC(O) |
| 20 | MeO | 2-Cl | 3 | —CH₂CH₂—N(Me)—CH₂CH₂— | | N(Me)₂ | n-PrOC(O) |
| 21 | Ph | 2-Cl | 3 | Me | Me | Me | CN |
| 22 | Ph | 2-Cl | 3 | Et | Et | CF₃ | MeC(O) |
| 23 | Ph | 2-Cl | 3 | n-Pr | n-Pr | c-Pr | PhC(O) |
| 24 | Ph | 2-Cl | 3 | Me | c-Pr | c-Hexyl | MeOC(O) |
| 25 | Ph | 2-Cl | 3 | Me | CF₃ | NH₂ | HN═CH |
| 26 | NH₂ | 2-Cl | 3 | Me | CH₂═CHCH₂— | N(Me)₂ | N(Me)═CH |
| 27 | NH₂ | 2-Me | 3 | Me | CH≡CCH₂— | H | 1-tetrazolyl |
| 28 | NH₂ | 2-Me | 3 | Me | Ph | H | HC(O) |
| 29 | NH₂ | 2-Me | 3 | Me | 2-Thienyl | H | EtC(O) |
| 30 | NH₂ | 2-Me | 3 | Me | 2-Furyl | H | n-PrOC(O) |
| 31 | N(Me)₂ | 2-Cl | 3 | Me | 2-Pyridyl | H | CN |
| 32 | N(Me)₂ | 2-Me | 3 | Me | 4-Piperidyl | H | MeC(O) |
| 33 | N(Me)₂ | 2-Me | 3 | —CH₂CH₂CH₂— | | H | PhC(O) |
| 34 | N(Me)₂ | 2-Me | 3 | —CH₂CH(Me)CH₂— | | H | MeOC(O) |
| 35 | N(Me)₂ | 2-Me | 3 | —CH₂CH₂CH₂CH₂— | | H | HN═CH |
| 36 | HN(Me)C(O) | 2-Cl | 3 | —CH₂CH₂CH₂CH₂CH₂— | | H | N(Me)═CH |
| 37 | HN(Me)C(O) | 2-CF₃ | 3 | —CH₂CH₂—O—CH₂CH₂— | | H | 1-tetrazolyl |
| 38 | HN(Me)C(O) | 2-CF₃ | 3 | —CH₂CH₂—C(O)—CH₂CH₂— | | H | HC(O) |
| 39 | HN(Me)C(O) | 2-CF₃ | 3 | —CH₂CH₂CH(OEt)CH₂CH₂— | | H | EtC(O) |
| 40 | HN(Me)C(O) | 2-CF₃ | 3 | —CH₂CH₂—N(Me)—CH₂CH₂— | | H | n-PrOC(O) |
| 41 | MeOC(O) | 2-Cl | 3 | Me | Me | H | CN |
| 42 | MeOC(O) | 2-CF₃ | 3 | Et | Et | H | MeC(O) |
| 43 | MeOC(O) | 2-CF₃ | 3 | n-Pr | n-Pr | H | PhC(O) |
| 44 | MeOC(O) | 2-CF₃ | 3 | Me | c-Pr | H | MeOC(O) |
| 45 | MeOC(O) | 2-CF₃ | 3 | Me | CF₃ | Me | HN═CH |
| 46 | c-Pentyloxy | 2-Cl | 3 | Me | CH₂═CHCH₂— | CF₃ | N(Me)═CH |
| 47 | c-Pentyloxy | 2-OH | 3 | Me | CH≡CCH₂— | c-Pr | 5-tetrazolyl |
| 48 | c-Pentyloxy | 2-OH | 3 | Me | Ph | c-Hexyl | HC(O) |
| 49 | c-Pentyloxy | 2-OH | 3 | Me | 2-Thienyl | NH₂ | EtC(O) |
| 50 | c-Pentyloxy | 2-OH - | 3 | Me | 2-Furyl | N(Me)₂ | n-PrOC(O) |
| 51 | MeS | 2-Cl | 5 | Me | Me | H | CN |
| 52 | CN | 2-MeO | 5 | Et | Et | H | MeC(O) |
| 53 | CF₃C(O) | 2-MeO | 5 | n-Pr | n-Pr | H | PhC(O) |
| 54 | 2-Furyl | 2-MeO | 5 | Me | c-Pr | H | MeOC(O) |
| 55 | H | 2-MeO | 5 | Me | CF₃ | H | HN═CH |
| 56 | Me | 2-Cl | 5 | Me | CH₂═CHCH₂— | H | N(Me)═CH |
| 57 | Me | 2-CF₃O | 5 | Me | CH≡CCH₂— | H | 1-tetrazolyl |
| 58 | Me | 2-CF₃O | 5 | Me | Ph | H | HC(O) |
| 59 | Me | 2-CF₃O | 5 | Me | 2-Thienyl | H | EtC(O) |
| 60 | Me | 2-CF₃O | 5 | Me | 2-Furyl | H | n-PrOC(O) |
| 61 | CF₃ | 2-Cl | 5 | Me | 2-Pyridyl | H | CN |
| 62 | CF₃ | 2-CF₃O | 5 | Me | 4-Piperidyl | H | MeC(O) |
| 63 | CF₃ | 2-CF₃O | 5 | —CH₂CH₂CH₂— | | Me | PhC(O) |
| 64 | CF₃ | 2-CF₃O | 5 | —CH₂CH(Me)CH₂— | | CF₃ | MeOC(O) |
| 65 | CF₃ | 2-CF₃O | 5 | —CH₂CH₂CH₂CH₂— | | c-Pr | HN═CH |
| 66 | MeO | 2-Cl | 5 | —CH₂CH₂CH₂CH₂CH₂— | | c-Hexyl | N(Me)═CH |
| 67 | MeO | 2-SH | 5 | —CH₂CH₂—O—CH₂CH₂— | | NH₂ | 1-tetrazolyl |
| 68 | MeO | 2-SH | 5 | —CH₂CH₂—C(O)—CH₂CH₂— | | N(Me)₂ | HC(O) |
| 69 | MeO | 2-SH | 5 | —CH₂CH₂CH(OEt)CH₂CH₂— | | H | EtC(O) |
| 70 | MeO | 2-SH | 5 | —CH₂CH₂—N(Me)—CH₂CH₂— | | H | n-PrOC(O) |
| 71 | Ph | 2-Cl | 6 | Me | Me | H | CN |
| 72 | Ph | 2-MeS | 6 | Et | Et | H | MeC(O) |
| 73 | Ph | 2-MeS | 6 | n-Pr | n-Pr | H | PhC(O) |
| 74 | Ph | 2-MeS | 6 | Me | c-Pr | H | MeOC(O) |
| 75 | Ph | 2-MeS | 6 | Me | CF₃ | H | HN═CH |
| 76 | NH₂ | 2-Cl | 6 | Me | CH₂═CHCH₂— | H | N(Me)═CH |
| 77 | NH₂ | 2-MeS(O)₂ | 6 | Me | CH≡CCH₂— | H | 5-tetrazolyl |
| 78 | NH₂ | 2-MeS(O)₂ | 6 | Me | Ph | H | HC(O) |
| 79 | NH₂ | 2-MeS(O)₂ | 6 | Me | 2-Thienyl | H | EtC(O) |
| 80 | NH₂ | 2-MeS(O)₂ | 6 | Me | 2-Furyl | H | n-PrOC(O) |
| 81 | N(Me)₂ | 2-Cl | 6 | Me | 2-Pyridyl | H | CN |
| 82 | N(Me)₂ | 2-NO₂ | 6 | Me | 4-Piperidyl | H | MeC(O) |
| 83 | N(Me)₂ | 2-NO₂ | 6 | —CH₂CH₂CH₂— | | H | PhC(O) |
| 84 | N(Me)₂ | 2-NO₂ | 6 | —CH₂CH(Me)CH₂— | | H | MeOC(O) |
| 85 | N(Me)₂ | 2-NO₂ | 6 | —CH₂CH₂CH₂CH₂— | | H | HN═CH |
| 86 | HN(Me)C(O) | 2-Cl | 6 | —CH₂CH₂CH₂CH₂CH₂— | | H | N(Me)═CH |
| 87 | HN(Me)C(O) | 2,5-diMe | 6 | —CH₂CH₂—O—CH₂CH₂— | | H | 1-tetrazolyl |
| 88 | HN(Me)C(O) | 2,5-diMe | 6 | —CH₂CH₂—C(O)—CH₂CH₂— | | H | HC(O) |
| 89 | HN(Me)C(O) | 2,5-diMe | 6 | —CH₂CH₂CH(OEt)CH₂CH₂— | | H | EtC(O) |
| 90 | HN(Me)C(O) | 2,5-diMe | 6 | —CH₂CH₂—N(Me)—CH₂CH₂— | | H | n-PrOC(O) |
| 91 | MeOC(O) | 2-Cl | 3 | Me | Me | Me | CN |
| 92 | MeOC(O) | 2,5-diCl | 3 | Et | Et | CF₃ | MeC(O) |
| 93 | MeOC(O) | 2,5-diCl | 3 | n-Pr | n-Pr | c-Pr | PhO(O) |
| 94 | MeOC(O) | 2,5-diCl | 3 | Me | c-Pr | c-Hexyl | MeOC(O) |
| 95 | MeOC(O) | 2,5-diCl | 3 | Me | CF₃ | NH₂ | HN═CH |
| 96 | c-Pentyloxy | 2-Cl | 3 | Me | CH₂═CHCH₂— | N(Me)₂ | N(Me)═CH |
| 97 | c-Pentyloxy | — | 3 | Me | CH≡CCH₂— | H | 1-tetrazolyl |
| 98 | c-Pentyloxy | — | 3 | Me | Ph | H | HC(O) |
| 99 | c-Pentyloxy | — | 3 | Me | 2-Thienyl | Et | EtC(O) |
| 100 | c-Pentyloxy | — | 3 | Me | 2-Furyl | H | n-PrOC(O) |

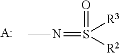

### 2) Production Method of the Compound of the Present Invention

The compound of the present invention can be produced by a well-known method, and also can be produced by the method described in Examples. An example of the production method of the compound of the present invention will be described below.

In the compound of the present invention, the compound represented by formula (I), wherein Q is Q1, Q2 or Q3 described above, R⁵ is hydrogen atom, and G is oxygen atom, can be produced by the method described in the formula below.

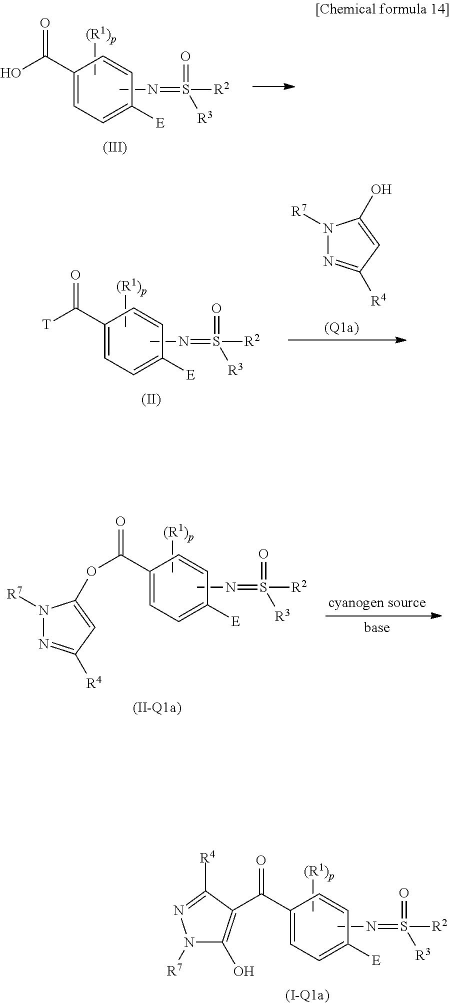

(In the formula, E, R¹ to R⁴, R⁷ and p are as defined above, T represents a halogen atom, an elimination group such as imidazolyl group or the like).

In the compound of the present invention, the compound represented by formula (I), wherein Q is Q1, Q2, Q3 described above, R⁵ in Q1, Q2 and Q3 represents a group other than hydrogen atom, G is oxygen atom, can be produced by the method described in the formula below.

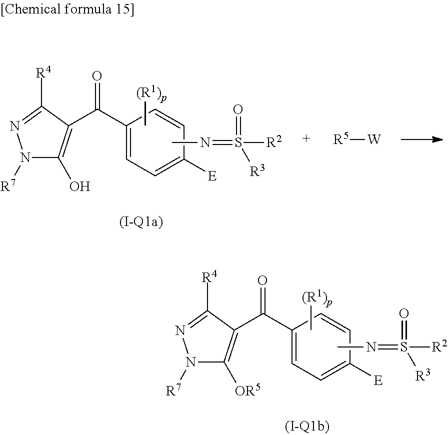

(In the formula, E, R¹ to R⁵, R⁷ and p are as defined above, W is an elimination group including a halogen atom such as fluorine atom, chlorine atom, bromine atom, iodine atom or the like; a sulfonyloxy group such as methane sulfonyloxy group, p-toluene sulfonyloxy group, trifluoromethane sulfonyloxy group or the like, an acyloxy group such as acetoxy group, benzoyloxy group or the like; and the like).

In the compound of the present invention, the compound represented by formula (I), wherein Q is Q1, Q2, Q3 described above, R⁵ in Q1, Q2 and Q3 represents a group other than hydrogen atom, G is sulfur atom, can be produced by the method described in the formula below.

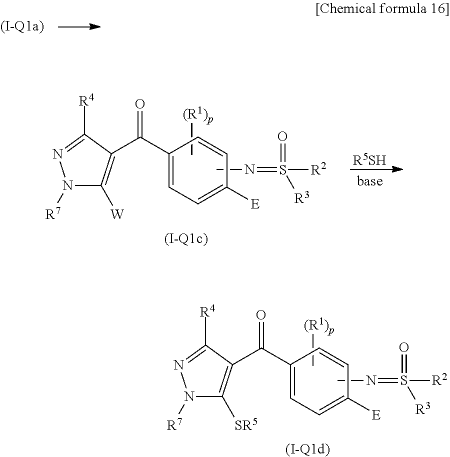

(In the formula, E, R¹ to R⁵, R⁷, W and p are as defined above).

In the compound of the present invention, the compound represented by formula (I), wherein Q is a group represented by Q8, can be produced by the method described in the formula below,

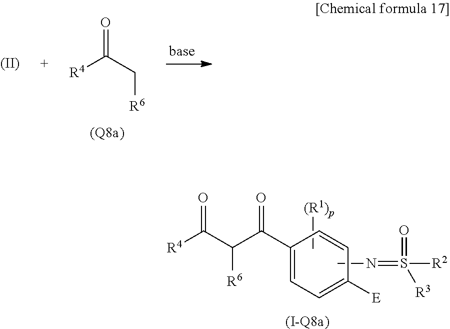

(In the formula, E, R¹ to R⁴, R⁶ and p are as defined above).

In the compound of the present invention, the compound represented by formula (I), wherein Q is a group represented by Q6, G is oxygen atom, can be produced by the method described in the formula below.

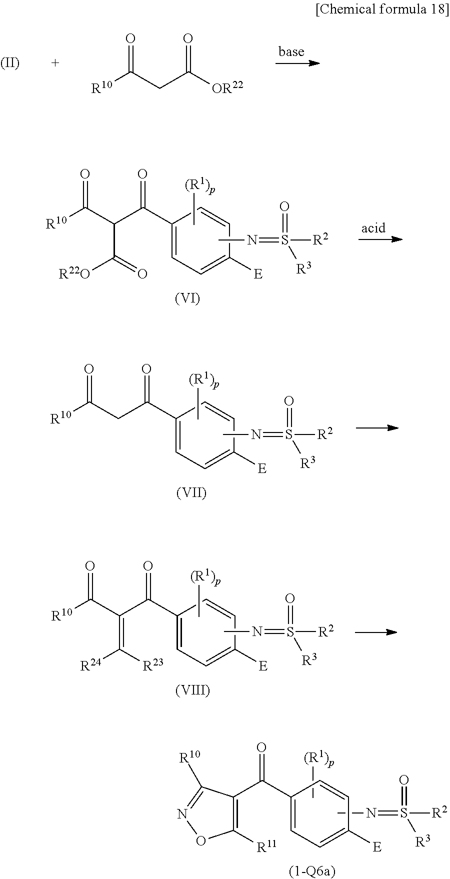

(In the formula, E, R¹ to R³, R¹⁰, R¹¹ and p are as defined above, R²² represent an alkyl group such as methyl group, ethyl group, n-propyl group, isopropyl group, t-butyl group or the like; a benzyl group; or a phenyl group, R²³ and R²⁴ each independently represents hydrogen atom, an alkyl group such as methyl group, ethyl group or the like; an alkoxy group such as methoxy group, ethoxy group, n-propoxy group or the like; a phenyl group; or a substituted or unsubstituted amino group such as amino group, dimethyl amino group, diethyl amino group or the like; provided that both R²³ and R²⁴ do not simultaneously represent an alkyl group and a phenyl group.

In the compound of the present invention, the compound represented by formula (I), wherein Q is a group represented by Q6, G is sulfur atom, can be produced by the method described in the formula below.

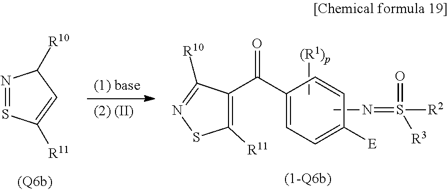

(In the formula, E, R¹ to R³, R¹¹ and p are as defined above).

The compound represented by formula (III) which is a raw material can be produced by the method described in the formula below.

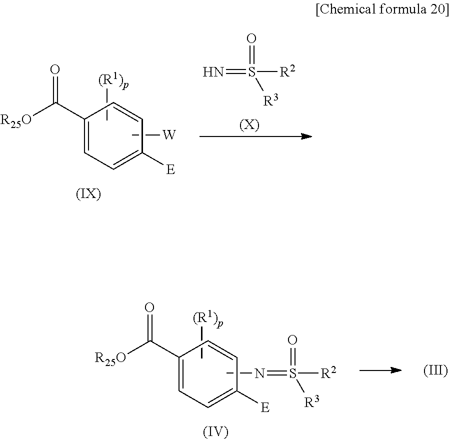

(In the formula, E, R¹ to R³, p and W are as defined above, R₂₅ represents an alkyl group having 1 to 6 carbon atoms; a haloalkyl group having 1 to 6 carbon atoms; or a phenyl group which is optionally substituted by an alkyl group having 1 to 3 carbon atoms, a haloalkyl group having 1 to 6 carbon atoms, an alkoxy group having 1 to 3 carbon atoms, an alkyl thio group having 1 to 3 carbon atoms, alkyl sulfonyl group having 1 to 3 carbon atoms, nitro group, cyano group, a halogen atom or the like; or the like).

Namely, the compound of the present invention can be produced by reacting benzoate ester (IX) with sulfoximine (X) by a well-known method to produce benzoate ester (IV), followed by hydrolyzing under the general hydrolysis conditions.

Benzoate ester (IX) which is a raw material can also be produced by a well-known method. In addition, this compound can also be synthesized by combining the general organic synthetic methods.

Sulfoximine (X) can also be synthesized by a well-known method.

In either of these reactions, if purification of the product is required after the completion of the reaction, known, commonly used purification means, such as distillation, recrystallization or column chromatography, can be employed following carrying out an ordinary post-treatment operation.

### 3) Herbicide

The compounds of the present invention (the compounds represented by formula (I) or salts thereof) exhibit high herbicidal activity in either soil treatment or foliar treatment under upland farming conditions; are effective on various upland weeds such as crabgrass, giant foxtail, velvetleaf, and pigweed; and also include compounds which exhibit selectivity toward crops such as corn, wheat or the like.

Moreover, the compounds of the present invention include compounds which exhibit plant growth-regulating activity such as retarding toward useful plants such as agricultural crops, ornamental plants, and fruit trees.

Additionally, the compounds of the present invention include compounds which have excellent exhibit herbicidal activity on various lowland weeds and which exhibit selectivity toward rice.

Furthermore, the compounds of the present invention can also be applied for controlling weeds in such places as fruit farms, lawns, railway track margins, and vacant lands.

The herbicide of the present invention includes one type, or two or more types of the compounds of the present invention as active ingredients. The herbicide of the present invention can be used in pure form without adding any other components to the compound of the present invention when applied practically, and also can be used, with an objective to use as agrochemicals, in the form which general agrochemicals may adopt, that is, wettable powder, granules, dusting powder, emulsifiable concentrates, water-soluble powder, suspending agent, flowable, or the like.

As additives and carriers, vegetable powders such as soy flour and wheat flour; fine mineral powder such as diatomaceous earth, apatite, gypsum, talc, bentonite, pyrophyllite, and clay; and organic and inorganic compounds such as sodium benzoate, urea, and sodium sulfate are used when solid formulation is required.

When a liquid formulation is required, petroleum fractions such as kerosene, xylene, and solvent naphtha, and cyclohexane, cyclohexanone, dimethylformamide, dimethyl sulfoxide, alcohol, acetone, trichloroethylene, methyl isobutyl ketone, mineral oil, vegetable oil, water, or the like, are used as a solvent.

Additionally, in order to achieve homogenous and stable forms in these formulations, it is also possible to add surfactants if necessary.

Although surfactants are not particularly limited, examples thereof include, for instance, nonionic surfactants such as alkylphenyl ether where polyoxyethylene is added, alkyl ether where polyoxyethylene is added, higher fatty acid ester where polyoxyethylene is added, sorbitan higher fatty acid ester where polyoxyethylene is added, and tristyryl phenyl ether where polyoxyethylene is added; sulfate ester of alkyl phenyl ether where polyoxyethylene is added, alkyl naphthalene sulfonate, polycarboxylate, lignin sulfonate, formaldehyde condensate of alkyl naphthalene sulfonate, and isobutylene-maleic anhydride copolymer.

Although concentrations of active ingredients in herbicides of the present invention vary depending on the aforementioned forms of formulation, in wettable powder for instance, the concentration of 5 to 90 weight % (hereinafter written simply as “%”) and preferably 10 to 85% is used; 3 to 70% and preferably 5 to 60% is used in emulsion; and 0.01 to 50% and preferably 0.05 to 40% is used in granules.

Wettable powder and emulsifiable concentrate obtained in this way, which are diluted to predetermined concentrations by water, are sprayed or mixed in soil as emulsion solution or suspension solution before or after the weed germination. When herbicides of the present invention are practically used, an adequate amount of active ingredients, which is 0.1 g or more per 1 hectare, is applied.

Herbicides of the present invention can also be used by mixing with known fungicides, insecticides, acaricides, other herbicides, plant growth regulators, fertilizers, antidotes or the like.

Especially, if it is used by mixing with the herbicides, the used amount of chemicals can be reduced.

In addition, not only labor saving but also a further higher effect can be expected due to synergism with mixed chemicals. In this case, combined use with a two or more of known herbicides is also possible.

Other active ingredients of the herbicide using in the present invention are not particularly limited. Examples of the active ingredients include the following (a) to (k).

(a) phenoxy type such as 2,4-D, 2,4-DB, 2,4-DP, MCPA, MCPB, MCPP, clomeprop or the like; aromatic carboxylic acid type such as 2,3,6-TBA, dicamba, chloramben, picloram, triclopyr, clopyralid, aminopyralid, fluoroxypyr or the like; others demonstrating their herbicidal effect by disturbance of plant hormone action, such as naptalam, benazolin, quinclorac, quinmerac, diflufenzopyr or the like;
(b) urea type such as chlorotoluron, diuron, fluometuron, linuron, isoproturon, tebuthiuron, isouron, siduron, chloroxuron, chlorobromuron, dimefuron, ethidimuron, fenuron, methabenzthiazuron, metobromuron, metoxuron, monolinuron, neburon or the like; triazine type such as simazine, atrazine, atratone, simetryn, prometryn, dimethametryn, hexazinone, metribuzin, metamitron, terbuthylazine, cyanazine, ametryn, cybutryne, propazine, desmetryne, prometon, terbumeton, terbutryne, trietazine or the like; uracil type such as bromacil, lenacil, terbacil or the like; anilide type such as propanil, pentanochlor or the like; carbamate type such as desmedipham, phenmedipham or the like; hydroxybenzonitrile type such as bromoxynil, ioxynil, bromofenoxim or the like; others demonstrating their herbicidal effect by inhibition of plant photosynthesis, such as pyridate, chloridazon, bentazon, amicarbazone, methazole, pyridafol or the like;
(c) quaternary ammonium salt type demonstrating their rapid herbicidal effect by allowing themselves to become a free radical and generate active oxygen, such as paraquat, diquat or the like;
(d) diphenyl ether type such a chlomethoxyfen, bifenox, acifluorfen-sodium, fomesafen, oxyfluorfen, lactofen, ethoxyfen-ethyl, fluoroglycofen-ethyl, halosafen or the like; cyclic imide type such as chlorphthalim, flumioxazin, flumiclorac-pentyl, cinidon-ethyl or the like; thiadiazole type such as fluthiacet-methyl, thidiazimin or the like; oxadiazole type such as oxadiargyl, oxadiazon or the like; triazolinone type such as azafenidin, sulfentrazone, carfentrazone-ethyl, bencarbazone or the like; phenyl pyrazole type such as fluazolate, pyraflufen-ethyl or the like; pyrimidine dione type such as benzfendizone, butafenacil or the like; others demonstrating their herbicidal effect by inhibition of chlorophyll biosynthesis to cause abnormal accumulation of photosensitized peroxidation in the plant body, such as pentoxazone, profluazol, pyrachlonil, flufenpyr-ethyl or the like;
(e) pyrazole type such as pyrazolynate, pyrazoxyfen, benzofenap, topramezone (BAS-670H), pyrasulfotole or the like; triketone type such as sulcotrione, mesotrione, tefuryltrione (AVH-301), tembotrione or the like; isoxazole type such as isoxaflutole, isoxachlortole or the like; others demonstrating their herbicidal effect by inhibition of biosynthesis of plant pigment such as carotenoid or the like to show whitening effect, such as amitrol, fluometuron, aclonifen, norflurazon, fluridone, flurtamone, diflufenican, clomazone, benzobicyclone, picolinafen, beflubutamid, fluorochloridone or the like;
(f) aryloxyphenoxypropionic acid type such as diclofop-methyl, flamprop-M-methyl, fluazifop-butyl, haloxyfop-methyl, quizalofop-ethyl, cyhalofop-butyl, fenoxaprop-ethyl, metamifop, clodinafop-propargyl, propaquizafop-P-ethyl or the like; cyclohexanedione type such as alloxydim-sodium, clethodim, sethoxydim, tralkoxydim, butroxydim, tepraloxydim, profoxydim, cycloxydim or the like; phenyl pyrazoline type such as pinoxaden or the like demonstrating their herbicidal effect by inhibition of acetyl-CoA carboxylase of plant;
(g) sulfonyl urea type such as chlorimuron-ethyl, sulfometuron-methyl, primisulfuron-methyl, bensulfuron-methyl, chlorsulfuron, metsulfuron-methyl, cinosulfuron, pyrazosulfuron-ethyl, azimsulfuron, flazasulfuron, rimsulfuron, nicosulfuron, imazosulfuron, cyclocyclosulfamuron, prosulfuron, flupyrsulfuron, halosulfuron-methyl, thifensulfuron-methyl, ethoxysulfuron, oxasulfuron, ethametsulfuron-methyl, iodosulfuron, sulfosulfuron, triasulfuron, tribenuron-methyl, tritosulfuron, foramsulfuron, trifloxysulfuron, mesosulfuron-methyl, orthosulfamuron, triflusulfuron-methyl, amidesulfuron, TH-547 or the like; triazolopyrimidine sulfonamide type such flumetsulam, metosulam, diclosulam, cloransulam-methyl, florasulam, metosulfam, penoxsulam, pyroxsulam or the like; imidazolinone type such as imazapyr, imazethapyr, imazaquin, imazamox, imazamethabenz, imazapic or the like; pyrimidinyl salicylic acid type such as pyrithiobac-sodium, bispyribac-sodium, pyriminobac-methyl, pyribenzoxim, pyriftalid, pyrimisulfan or the like; sulfonyl aminocarbonyl triazolinone type such as flucarbazone, propoxycarbazone, thiencarbazone-methyl or the like; others demonstrating their herbicidal effect by inhibition of plant amino-acid biosynthesis, such as glyphosate, glyphosate-ammonium, glyphosate-isopropylamine, sulfosate, glufosinate, glufosinate-ammonium, bilanafos or the like;
(h) dinitroaniline type such as trifluralin, oryzalin, pendimethalin, ethalfluralin, benfluralin, prodiamine, butralin, dinitramine or the like; benzamide type such as pronamide, tebutam or the like; organic phosphorus type such as amiprofos-methyl, butamifos or the like; phenyl carbamate type such as propham, chlorpropham, carbetamide or the like; pyridine type such as dithiopyr, thiazopyr or the like; others demonstrating their herbicidal effect by inhibition of plant cell mitosis, such as DCPA or the like;
(i) chloroacetamide type such as alachlor, metazachlor, butachlor, pretilachlor, metolachlor, S-metolachlor, thenylchlor, pethoxamid, acetochlor, propachlor, propisochlor, dimethenamid, dimethenamid-P, dimethachlor or the like; acetamide type such as diphenamid, napropamide, naproanilide or the like; oxyacetamide type such as flufenacet, mefenacet or the like; others demonstrating their herbicidal effect by inhibition of plant cell devision or inhibition of very long chain fatty acid biosynthesis such as fentrazamide, cafenstrole, indanofan, anilofos, piperophos or the like;
(j) thiocarbamate type such as molinate, dimepiperate, EPTC, butylate, cycloate, esprocarb, orbencarb, pebulate, prosulfocarb, thiobencarb, tiocarbazil, triallate, vernolate or the like; benzofuran type such as benfuresate, ethofumesate or the like; others demonstrating their herbicidal effect by inhibition of plant lipid biosynthesis, such as bensulide, TCA, dalapon, flupropanate or the like;
(k) other herbicides such as asulam, DNOC, dinoseb, dinoterb, flupoxam, dichlobenil, chlorthiamid, isoxaben, quinclorac, MSMA, DSMA, endothall, sodiumchlorate, pelargonic acid, fosamine, flamprop-isopropyl, difenzoquat, bromobutide, chlorflurenol, cinmethylin, cumyluron, dazomet, daimuron, methyl-dymron, etobenzanid, matam, oxaziclomefone, oleic acid, pyributicarb, pyroxasulfone (KIH-485), HOK-201 or the like.

### EXAMPLES

Next, the present invention will be described in more detail using Examples and Reference examples. However, the present invention is not limited by Examples and Reference examples.

### (1) Synthesis of Precursor

### Reference Example 1

### 2-chloro-3-(1-oxothianylidene amino)terephthalic acid 4-methyl ester

### Step 1) Synthesis of 2-chloro-3-dimethoxymethyl phenol

23.1 g of 2-chloro-3-hydroxybenzaldehyde was dissolved in 150 ml of methanol, and 20.2 g of orthoformic acid trimethyl was added to the resulting solution, followed by 0.5 ml of hydrochloric acid. The resulting reaction solution was heated under reflux for one night and concentrated under reduced pressure. 100 ml of sodium bicarbonate water was added to the residue, and extracted with 200 ml of ethyl acetate twice. The organic layer was washed with brine, dried with magnesium sulfate, filtered, concentrated to obtain 35.1 g (yield: 100%) of 2-chloro-3-dimethoxymethyl phenol which is colorless and oily.

### Step 2) Synthesis of 4-bromo-2-chloro-3-hydroxybenzaldehyde

35.1 g of 2-chloro-3-dimethoxymethyl phenol was dissolved in 200 ml of chloroform, and 30 ml of chloroform solution of bromine including 23.5 g bromine was dropped slowly into the resulting solution under ice-cold conditions, followed by stirring for one night at room temperature. 300 ml of 5% aqueous sodium hydrogen sulfite solution was added to the resulting reaction solution under ice-cold conditions, and extracted with 300 ml of chloroform twice. The organic layer was washed with brine, dried with magnesium sulfate, filtered, concentrated to obtain 19.8 g (yield: 57.2%) of 4-bromo-2-chloro-3-hydroxybenzaldehyde which is slightly yellow and crystalline.

### Step 3) Synthesis of 6-bromo-2-chloro-3-dimethoxymethyl phenol

10 g of 4-bromo-2-chloro-3-hydroxybenzaldehyde was dissolved in 66 ml of methanol, and 5.4 g of orthoformic acid trimethyl was added to the resulting solution, followed by catalyst quantity of hydrochloric acid. The resulting reaction solution was heated under reflux for one night and concentrated under reduced pressure. 50 ml of sodium bicarbonate water was added to the residue, and extracted with 100 ml of ethyl acetate twice. The organic layer was washed with brine, dried with magnesium sulfate, filtered, concentrated to obtain 12.6 g (yield: 100%) of 6-bromo-2-chloro-3-dimethoxymethyl phenol which is colorless and oily.

### Step 4) Synthesis of 3-chloro-4-dimethoxymethyl-2-hydroxybenzoic acid methyl

4.0 g of 6-bromo-2-chloro-3-dimethoxymethyl phenol was dissolved in 35.5 ml of methanol, and 1.4 g of acetic acid sodium, 0.31 g of 1,1′-bis(diphenyl phosphino)ferrocene and 0.12 g of acetic acid(II)palladium were added to the resulting solution in autoclave. The autoclave was filled with carbon monoxide so that the inner pressure was 0.85 MPa, and the reaction solution was heated at 90 to 100° C. for 4 hours. After releasing the inner pressure, the resulting reaction solution was immersed in 50 ml of water, and extracted with 100 ml of ethyl acetate twice. The organic layer was washed with brine, dried with magnesium sulfate, filtered, concentrated to obtain 3.9 g (yield: 100%) of 3-chloro-4-dimethoxymethyl-2-hydroxybenzoic acid methyl which is oily and slightly yellow. The obtained compound was used to the next reaction without performing purification.

### Step 5) Synthesis of 3-chloro-4-dimethoxymethyl-2-trifluoromethane sulfonyloxybenzoic acid methyl

The residue was dissolved in 30 ml of methylene chloride. In ice-cold conditions, 1.86 g of triethylamine and 4.4 g of trifluoromethane sulfonic acid anhydride were added to the resulting solution in this order. The resulting reaction solution was stirred at room temperature for one night, and concentrated under reduced pressure, then added with water, and extracted with 100 ml of ethyl acetate twice. The organic layer was washed with brine, dried with magnesium sulfate, filtered, and concentrated. The resulting residue was purified by silica gel column chromatography (eluent: ethyl acetate/n-hexane=1/9) to obtain 5.29 g (yield: 94.8%) of 3-chloro-4-dimethoxymethyl-2-trifluoromethane sulfonyloxybenzoic acid methyl which is colorless and oily.

### Step 6) Synthesis of 3-chloro-4-dimethoxymethyl-2-(1-oxothianylidene amino)benzoic acid methyl

2.5 g of 3-chloro-4-dimethoxymethyl-2-trifluoromethane sulfonyloxybenzoic acid methyl was dissolved in 30 ml of toluene, and 1.10 g of 1-iminothiane-1-one, 3.10 g of cesium carbonate and 0.37 g of 4,5-bis(diphenyl phosphino)-9,9-dimethyl xanthene were added to the resulting solution. The reaction system was replaced with nitrogen gas after deaerating under reduced pressure. 0.29 g of tris(dibenzylidene acetone)dipalladium was added to the resulting solution under nitrogen atmosphere, and further sufficient nitrogen gas replacement was performed. The resulting reaction solution was heated under reflux for 3 hours. Next, the solution was cooled to room temperature, and added with 200 ml of ethyl acetate and 100 ml of water, filter the insoluble matter using Celite. Celite was washed with 100 ml of ethyl acetate and 100 ml of water, and extracted the solution. The organic layer was washed with brine, dried with magnesium sulfate, filtered, and concentrated. The residue was purified by silica gel column chromatography (eluent: ethyl acetate/n-hexane=2/1) to obtain 2.43 g (yield: 100%) of 3-chloro-4-dimethoxymethyl-2-(1-oxothianylidene amino)benzoic acid methyl which is yellow-colored and amorphous.

### Step 7) Synthesis of 2-chloro-3-(1-oxothianylidene amino)terephthalic acid 4-methyl ester

3.49 g of 3-chloro-4-dimethoxymethyl-2-(1-oxothianylidene amino)benzoic acid methyl was dissolved in 23 ml of tetrahydrofuran, and 10% hydrochloric acid was added to the resulting solution. The solution was heated for 1.5 hours at 60° C. Next, the resulting reaction solution was cooled to room temperature and concentrated under reduced pressure. 100 ml of water was added to the residue, and extracted with 200 ml of ethyl acetate twice. The organic layer was washed with brine, dried with magnesium sulfate, filtered, and concentrated to obtain 3.03 g (yield: 99.8%) of 3-chloro-4-formyl-2-(1-oxothianylidene amino)benzoic acid methyl which is slightly yellow and amorphous. The obtained compound was used to the next reaction without performing purification.

3-chloro-4-formyl-2-(1-oxothianylidene amino)benzoic acid methyl was dissolved in 17 ml of tetrahydrofuran, and 18.6 ml of amide sulfuric acid aqueous solution including 1.15 g of amide sulfuric acid was dropped into the resulting solution under ice-cold conditions. The solution was stirred at the same temperature for 15 minutes, and 5 ml of sodium chlorite aqueous solution including 1.07 g of sodium chlorite was dropped into the solution. The solution was stirred at room temperature 1 hour. Next, 80 ml of water was added to the resulting reaction solution, and extracted with 150 ml of ethyl acetate twice. The organic layer was washed with 5% sodium bisulfite aqueous and brine, dried with magnesium sulfate, filtered, concentrated to obtain 1.96 g (yield: 56.6%) of 2-chloro-3-(1-oxothianylidene amino)terephthalic acid 4-methyl ester which is brown-colored and amorphous.

### Reference Example 2

### 2-chloro-4-methoxymethyl-3-(1-oxothianylidene amino)benzaldehyde

### Step 1) Synthesis of [3-chloro-4-dimethoxymethyl-2-(1-oxothianylidene amino)phenyl]methanol

1.84 g of 3-chloro-4-dimethoxymethyl-2-(1-oxothianylidene amino)benzoic acid methyl was dissolved in 26 ml of toluene. The resulting solution was cooled to −78° C., and 10.5 ml of 1.0 M toluene solution of diisobutyl aluminium hydride was slowly dropped into the solution. The resulting solution was stirred at the same temperature for 50 minutes, and stirred at 0° C. for 40 minutes, and further stirred at room temperature for 1.5 hours. Next, the resulting reaction solution was poured into ice, and the gel-like material generated was removed by Celite filtration, followed by washing the Celite with 200 ml of ethyl acetate. After concentrating the filtrate under reduced pressure, 100 ml of water added to the residue, and extracted with 200 ml of ethyl acetate twice. The organic layer was washed with brine, dried with magnesium sulfate, filtered, concentrated to obtain 1.37 g (yield: 82.0%) of [3-chloro-4-dimethoxymethyl-2-(1-oxothianylidene amino)phenyl]methanol which is colorless and amorphous. The obtained compound was used to the next reaction without performing purification.

### Step 2) Synthesis of 2-chloro-4-methoxymethyl-3-(1-oxothianylidene amino)benzaldehyde

0.3 g of [3-chloro-4-dimethoxymethyl-2-(1-oxothianylidene amino)phenyl]methanol was dissolved in 5 ml of tetrahydrofuran, and 0.03 g of sodium hydrate was added to the resulting solution under ice-cold conditions, followed by stirring for 25 minutes. Next, the solution was added with 0.16 g of methyl iodide, and stirred at room temperature for one night. Next, 100 ml of water was added to the resulting reaction solution, and extracted with 30 ml of ethyl acetate twice. The organic layer was washed with brine, dried with magnesium sulfate, filtered, concentrated to obtain 0.38 g of (2-chloro-3-dimethoxymethyl-6-methoxymethyl phenyl)-(1-oxothianylidene)amine which is slight yellow and amorphous. The obtained compound was used to the next reaction without performing purification.

0.38 g of (2-chloro-3-dimethoxymethyl-6-methoxymethyl phenyl)-(1-oxothianylidene)amine was dissolved in 2.3 ml of tetrahydrofuran, and 0.9 ml of 10% hydrochloric acid was added to the resulting solution. The solution was heated at 60° C. for 1.5 hours. Next, the solution was cooled to room temperature, and concentrated under reduced pressure. 10 ml of water was added to the residue, and extracted with 30 ml of ethyl acetate twice. The organic layer was washed with brine, dried with magnesium sulfate, filtered, concentrated to obtain 0.3 g (yield: 100%) of 2-chloro-4-methoxymethyl-3-(1-oxothianylidene amino)benzaldehyde which is slightly yellow and amorphous.

### Reference Example 3

### 2-chloro-4-cyano-3-hydroxybenzoic acid methyl

### Step 1) Synthesis of 4-bromo-2-chloro-3-hydroxybenzoic acid methyl

5 g of 4-bromo-2-chloro-3-hydroxybenzaldehyde was dissolved in 30 ml of tetrahydrofuran, and 47.5 ml of amide sulfuric acid aqueous solution including 2.68 g of amide sulfuric acid was dropped into the resulting solution at room temperature. The solution was stirred at the same temperature for 20 minutes. 12 ml of sodium chlorite aqueous solution including 2.5 g of sodium chlorite was dropped into the resulting solution, and the solution was stirred at room temperature for 3.5 hours. Next, 80 ml of water added to the resulting reaction solution, and extracted with 200 ml of ethyl acetate twice. The organic layer was washed with 5% sodium bisulfate aqueous and brine, dried with magnesium sulfate, filtered, concentrated to obtain 5.8 g of 4-bromo-2-chloro-3-hydroxybenzoic acid which is slightly yellow and crystalline. The obtained compound was used to the next reaction without performing purification.

5.8 g of 4-bromo-2-chloro-3-hydroxybenzoic acid was dissolved in 120 ml of methanol, and 1 ml of sulfuric acid was added to the resulting solution. The solution was heated under reflux for 7.5 hours. Next, the resulting reaction solution was concentrated under reduced pressure, and 200 ml of saturated sodium bicarbonate water was added to the residue, and extracted with 300 ml of ethyl acetate twice. The organic layer was washed with brine, dried with magnesium sulfate, filtered, concentrated to obtain 4.99 g (yield: 88.6%) of 4-bromo-2-chloro-3-hydroxybenzoic acid methyl which is oily and slightly yellow.

### Step 2) Synthesis of 4-bromo-2-chloro-3-methoxybenzoic acid methyl

4.5 g of 4-bromo-2-chloro-3-hydroxybenzoic acid methyl was dissolved in 18 ml of N,N-dimethyl formamide, and 2.8 g of potassium carbonate and 3.6 g of methyl iodide were added to the resulting solution at room temperature. The solution was stirred at room temperature for one night. Next, the resulting reaction solution was filtered using Celite and the obtained filtrate was concentrated under reduced pressure, 150 ml of water was added to the residue, and extracted with 250 ml of ethyl acetate twice. The organic layer was washed with brine, dried with magnesium sulfate, filtered, concentrated. The residue was purified by silica gel column chromatography (eluent: ethyl acetate/n-hexane=1/9) to obtain 4.36 g (yield: 92.2%) of 4-bromo-2-chloro-3-methoxybenzoic acid methyl which is oily and slightly yellow.

### Step 3) Synthesis of 2-chloro-4-cyano-3-methoxybenzoic acid methyl

4.3 g of 4-bromo-2-chloro-3-methoxybenzoic acid methyl was dissolved to 76 ml of N,N-dimethyl acetamide, and 1.08 g of zinc cyanide, 0.70 g of tris(dibenzylidene acetone)dipalladium, and 0.72 g of 1,1′-bis(diphenyl phosphino)ferrocene were added to the resulting solution. The solution was heated under reflux for 2 hours. Next, the resulting reaction solution was cooled to room temperature, and filtered using Celite. Celite was washed with 200 ml of ethyl acetate, and the filtrate was concentrated under reduced pressure. 200 ml of water was added to the residue, and extracted with 300 ml of ethyl acetate twice. The organic layer was washed with brine, dried with magnesium sulfate, filtered, concentrated. The residue was purified by silica gel column chromatography (eluent: ethyl acetate/n-hexane=1/19) to obtain 3.08 g (yield: 89.2%) of 2-chloro-4-cyano-3-methoxybenzoic acid methyl which is slightly green and solid.

### Step 4) Synthesis of 2-chloro-4-cyano-3-hydroxybenzoic acid methyl

2.68 g of 2-chloro-4-cyano-3-methoxybenzoic acid methyl was dissolved in 70 ml of methylene chloride, and 11.8 ml of 1.0 M boron tribromide-methylene chloride solution was slowly dropped into the resulting solution at −10° C. After dropping, the solution was stirred at 0° C. for 30 minutes, and stirred at room temperature for a whole day and night. Next, 80 ml of saturated sodium bicarbonate water was added to the resulting reaction solution, and extracted with 100 ml of chloroform. The water layer was acidized using hydrochloric acid, and extracted with 100 ml of chloroform twice. The organic layer was dried with magnesium sulfate, filtered, concentrated to obtain 1.42 g (yield: 56.8%) of 2-chloro-4-cyano-3-hydroxybenzoic acid methyl which is white and solid.

### Reference Example 4

### 2-chloro-3-(1-oxothianylidene amino)-4-phenyl benzoic acid methyl

0.81 g of 4-bromo-2-chloro-3-(1-oxothianylidene amino)benzoic acid methyl was added to 10 ml of toluene, and replaced with nitrogen gas after deaerating under reduced pressure. 0.25 g of tetrakistriphenyl phosphine palladium was added to the resulting solution under nitrogen atmosphere, and further sufficient nitrogen gas replacement was performed. The resulting reaction solution was added to 0.52 g of phenyl boronic acid and 2 ml of 2M sodium carbonate aqueous solution, and heated under reflux for a whole day and night. Next, the solution was cooled to room temperature, and added to 200 ml of ethyl acetate and 100 ml of water to filter insoluble matter using Celite. Celite was washed with 300 ml of ethyl acetate and 300 ml of water, and extracted the solution. The organic layer was washed with brine, dried with magnesium sulfate, filtered, concentrated. The residue was purified by silica gel column chromatography (eluent: ethyl acetate/n-hexane=1/1) to obtain 0.57 g (yield: 71%) of 2-chloro-3-(1-oxothianylidene amino)-4-phenyl benzoic acid methyl which is yellow-colored and amorphous.

### Reference Example 5

### 2,4-dimethyl-3-(1-oxothianylidene amino)benzoic acid

1.21 g of 4-bromo-2-methyl-3-(1-oxothianylidene amino)benzoic acid was added to 20 ml of dioxane, and replaced with nitrogen gas after deaeration under reduced pressure. 1.78 g of potassium carbonate and 0.39 g of tetrakistriphenyl phosphine palladium were added to the resulting solution, and further sufficient nitrogen gas replacement was performed. The resulting reaction solution was added with 0.81 g of trimethyl boroxin and heated under reflux for a whole day and night. Next, the solution was cooled to room temperature, and added with 200 ml of ethyl acetate and 100 ml of water and filtered insoluble matter using Celite. Celite was washed with 300 ml of ethyl acetate and 300 ml of water, and extracted the solution. The organic layer was washed with brine, dried with magnesium sulfate, filtered, concentrated. The residue was purified by silica gel column chromatography (eluent: ethyl acetate/n-hexane=1/1) to obtain 0.74 g (yield: 74%) of 2,4-dimethyl-3-(1-oxothianylidene amino)benzoic acid which is yellow-colored and amorphous.

### (2) Synthesis of the Compound of the Present Invention

### Example 1

### 2-chloro-1-[(5-hydroxy-1-methyl pyrazole-4-yl)carbonyl]-4-methoxy-3-(1-oxothianylidene amino)benzene

### Step 1) Synthesis of 2-chloro-4-methoxy-3-(1-oxothianylidene amino)benzoic acid methyl

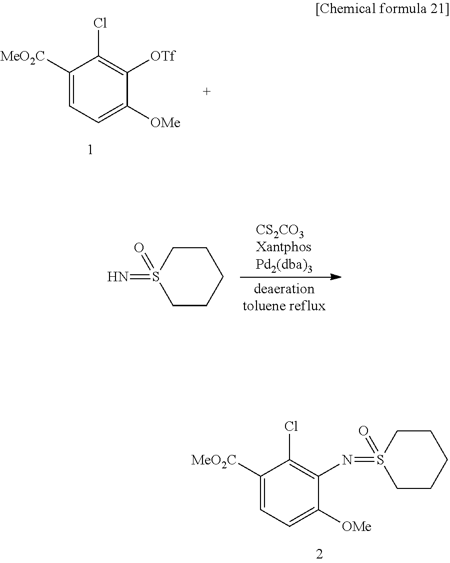

4.52 g of 2-chloro-4-methoxy-3-(trifluoromethyl sulfonyloxy)benzoic acid methyl 1 was added with 1.90 g of 1-iminothiane-1-one, 5.07 g of cesium carbonate, 0.37 g of 4,5-bis(diphenyl phosphino)-9,9-dimethyl xanthene and 100 ml of toluene, and replaced with nitrogen gas after deaerating under reduced pressure. The resulting solution was added with 0.30 g of tris(dibenzylidene acetone)dipalladium under nitrogen atmosphere, and further sufficient nitrogen gas replacement was performed. The resulting reaction solution was heated under reflux for a whole day and night. Next, the solution was cooled to room temperature, and added with 200 ml of ethyl acetate and 100 ml of water and filtered insoluble matter using Celite. Celite was washed with 300 ml of ethyl acetate and 300 ml of water, and extracted the solution. The organic layer was washed with brine, dried with magnesium sulfate, filtered, concentrated. The residue was purified by silica gel column chromatography (eluent: ethyl acetate/n-hexane=3/7) to obtain 0.96 g (yield: 22%) of 2-chloro-4-methoxy-3-(1-oxothianylidene amino)benzoic acid methyl 2 which is yellow-colored and amorphous.

### Step 2) Synthesis of 2-chloro-4-methoxy-3-(1-oxothianylidene amino)benzoic acid

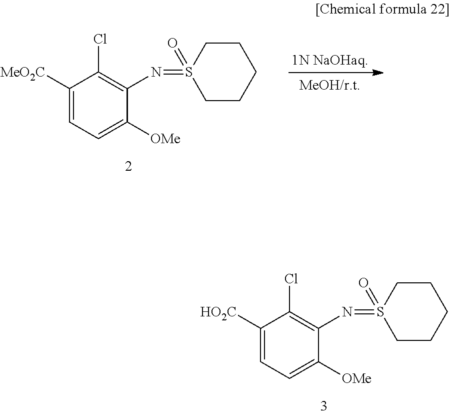

0.96 g of 2-chloro-4-methoxy-3-(1-oxothianylidene amino)benzoic acid methyl 2 was added with 15 ml of methanol to dissolve. The resulting solution was added with 15 ml of 1N sodium hydrate at room temperature and stirred at room temperature for a whole day and night to concentrate methanol (approximately 5 ml). The resulting solution was added with 50 ml of ice water and adjusted to pH 1 by concentrated hydrochloric acid, and extracted with 50 ml of ethyl acetate twice. The organic layer was washed with 30 ml of brine, dried with magnesium sulfate, filtered, concentrated to obtain 0.55 g (yield: 60%) of 2-chloro-4-methoxy-3-(1-oxothianylidene amino)benzoic acid 3 which is white and crystalline.

### Step 3) Synthesis of 2-chloro-1-[(5-hydroxy-1-methyl pyrazole-4-yl)carbonyl]-4-methoxy-3-(1-oxothianylidene amino)benzene

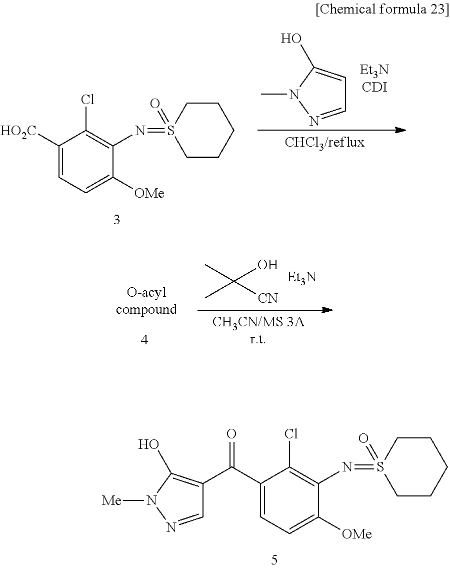

0.45 g of 2-chloro-4-methoxy-3-(1-oxothianylidene amino)benzoic acid 3 was added to 10 ml of chloroform, 0.37 g of 1,1′-carbonyl diimidazole in this order at room temperature. The resulting solution was stirred at room temperature for 1 hour. After ripening, the resulting solution was added with 0.17 g of N-methylpyrazolone and 0.17 g of triethylamine, and heated under reflux for 1 hour. Next, the resulting reaction solution was cooled and the solvent was concentrated. The residue was added with 10 ml of acetonitrile, 0.01 g of acetone cyanohydrin and 0.36 g of triethylamine, and stirred at room temperature for a whole day and night. Next, the insoluble matter was filtered, and the filtrate was concentrated. The residue was dissolved in 100 ml of chloroform, and washed with 100 ml of 1N hydrochloric acid, followed by water (100 ml). The organic layer was dried with magnesium sulfate, filtered, concentrated. The residue was purified by silica gel column chromatography (eluent: chloroform/methanol=19/1) to obtain 0.31 g (m.p. 89-91° C., yield: 55%) of 2-chloro-1-[(5-hydroxy-1-methyl pyrazole-4-yl)carbonyl)-4-methoxy-3-(1-oxothianylidene amino)benzene 5 which is white and crystalline.

### Example 2

### 2-chloro-4-methoxy-1-{[1-methyl-5-(2-naphthyl methoxy)pyrazole-4-yl]carbonyl}-3-(1-oxothianylidene amino)benzene

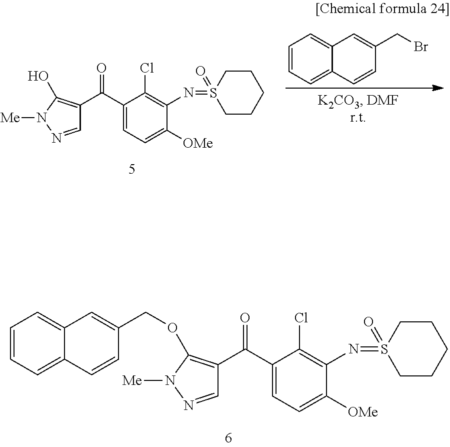

0.20 g of 2-chloro-1-[(5-hydroxy-1-methyl pyrazole-4-yl)carbonyl]-4-methoxy-3-(1-oxothianylidene amino)benzene 5 was dissolved in 2 ml of N,N-dimethyl formamide, and 0.08 g of potassium carbonate was added to the resulting solution. The solution was added with 0.12 g of 2-naphthyl methyl bromide, and stirred at room temperature for 1 hour. Water and ethyl acetate were added in the resulting reaction solution, and the organic layer was washed with brine, dried with magnesium sulfate, filtered, concentrated. The residue was purified by silica gel column chromatography (eluent: ethyl acetate/n-hexane=3/7) to obtain 0.11 g (yield: 41%) of 2-chloro-4-methoxy-1-{[1-methyl-5-(2-naphthyl methoxy)pyrazole-4-yl]carbonyl}-3-(1-oxothianylidene amino)benzene 6 which is white and amorphous. NMR of the obtained compound is shown below:

¹H-NMR (CDCl₃): 1.66 (m, 2H), 2.11 (m, 4H), 3.19 (m, 2H), 3.35 (m, 2H), 3.51 (s, 3H), 3.90 (s, 3H), 5.70 (s, 2H), 6.82 (d, 1H), 7.01 (d, 1H), 7.34 (s, 1H), 7.49-7.55 (m, 3H), 7.80-7.86 (m, 4H)

### Examples 3 to 260

The following compounds were synthesized by well-known production methods as Example 1.

TABLE 65

| Example | Structural formula | Physical property |
|---|---|---|
| 3 | 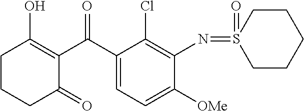 | amorphous ¹H-NMR(CDCl₃): 1.63 (m, 2H), 2.06 (m, 6H), 2.43(t, 2H), 2.74(t, 2H), 3.16 (m, 2H), 3.31 (m, 2H), 3.88 (s, 3H), 6.83(d, 1H), 6.86 (d, 1H) |
| 4 | 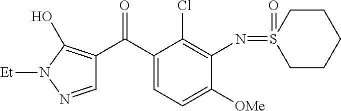 | m.p.: 137-139 |
| 5 | 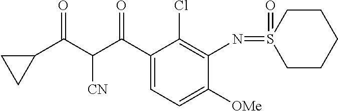 | m.p.: 154-156 |
| 6 | 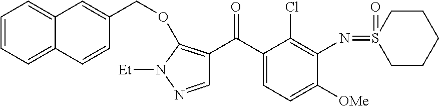 | amorphous ¹H-NMR (CDCl₃): 1.21 (t, 3H), .66 (m, 2H), 2.11 (m, 4H), 3.20 (m, 2H); 3.36 (m, 2H), 3.89 (q, 2H), 3.90 (s; 3H), 5.70 (s,2H), 6.82 (d, 1H),7.02 (d, 1H), 7.36 (s,1H), 7.47-7.56 (m, 3H), 7.80-7.87 (m, 4H) |
| 7 | 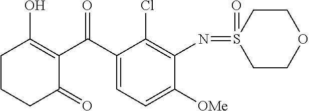 | amorphous ¹H-NMR (CDCl₃): 2.04 (m, 2H), 2.44 (t, 2H), 2.75 (t, 2H), 3.28(m, 2H), 3.43(m, 2H), 3.89 (s, 3H), 4.17 (m, 4H), 6.85 (d, 1H), 6.88 (d, 1H) |
| 8 | 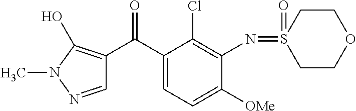 | amorphous ¹H-NMR (CDCl₃): 3.32 (m, 2H), 3.46 (m, 2H), 3.70 (s, 3H), 3.93(s, 3H), 4.20 (m, 4H), 6.87 (d, 1H), 7.14 (d, 1H),7.43 (s, 1H) |
| 9 | 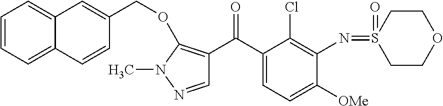 | amorphous ¹H-NMR (CDCl₃): 3.31 (m, 2H), 3.48 (m, 2H), 3.52 (s, 3H), 3.91 (s, 3H), 4.16 (m, 4H), 5.70 (s, 2H), 6.84 (d, 1H), 7.03 (d, 1H), 7.35 (s, 1H), 7.47-7.55 (m, 3H), 7.80-7.87 (m, 4H) |
| 10 | 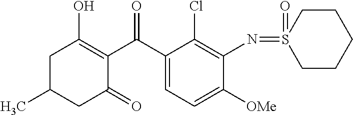 | amorphous ¹H-NMR (CDCl₃): 1.11 (d, 3H), 1.64 (m, 2H), 2.11 (m, 6H), 2,47(m, 2H), 2.78 (m, 1H), 3.17 (m, 2H), 3.31 (m, 2H), 3,88 (s, 3H), 6.83 (d, 1H), 6.86 (d, 1H)? |

TABLE 66

| | | |
|---|---|---|
| 11 | 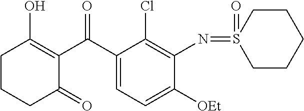 | amorphous ¹H-NMR (CDCl₃): 1.44 (t, 6H), 2.03 (m, 2H), 2.43 (t, 2H), 2.74 (t, 2H), 3.22 (m, 4H), 3.88 (s, 3H), 6.82 (d, 1H), 6.88 (d, 1H) |
| 12 | 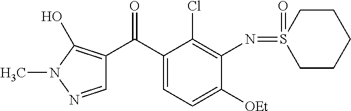 | m.p.: 88-90 |
| 13 | 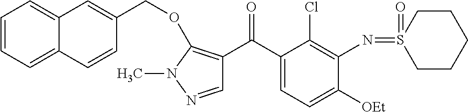 | amorphous ¹H-NMR (CDCl₃): 1.50 (t, 3H), 1.65 (m, 2H), 2.13 (m, 4H), 3.19 (m, 2H), 3.38 (m, 2H), 3.51 (s, 3H), 4.13 (q, 2H), 5.69 (s, 2H), 6.80 (d, 1H), 6.98 (d, 1H), 7.35 (s, 1H), 7.47-7.55 (m, 3H), 7.80-7.86 (m, 4H) |
| 14 | 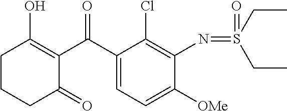 | amorphous ¹H-NMR (CDCl₃): 1.44 (t, 6H), 2.03 (m, 2H), 2.43 (t, 2H), 2.74 (t, 2H), 3.22 (m, 4H), 3.88 (s, 3H), 6.82 (d, 1H), 6.88 (d, 1H) |
| 15 | 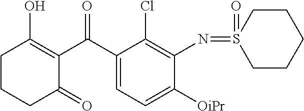 | amorphous 1H-NMR (CDCl3) 1.39 (d, 6H), 1.61 (m, 2H), 2.08 (m, 6H), 2.43 (t, 2H), 2.73 (t, 2H), 3.15 (m, 2H), 3.36 (m, 2H), 4.62 (m, 1H), 6.81 (d, 1H), 6.84 (d, 1H) |
| 16 | 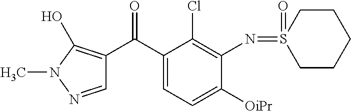 | m.p.: 139-141 |
| 17 | 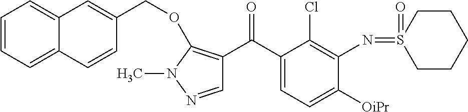 | amorphous 1H-NMR (CDCl3) 1.41 (d, 6H), 1.64 (m, 2H), 2.10 (m, 4H), 3.18 (m, 2H), 3.37 (m, 2H), 3.51 (s, 3H), 4.65 (m, 1H), 5.69 (s, 2H), 6.81 (d, 1H), 6.97 (d, 1H), 7.35 (s, 1H), 7.46-7.55 (m, 3H), 7.80- 7.86 (m, 4H) |
| 18 | 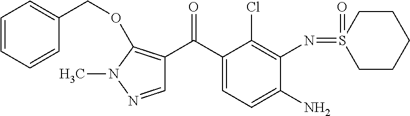 | amorphous ¹H-NMR (CDCl₃): 1.78 (m, 2H), 2.06 (m, 2H), 2.17 (m, 2H), 3.28 (m, 4H), 3.51 (s, 3H), 4.63 (br, 2H), 5.46 (s, 2H), 6.60 (d, 1H), 6.99 (d, 1H), 7.37 (m, 5H), 7.39 (s, 1H) |

TABLE 67

| | | |
|---|---|---|
| 19 | 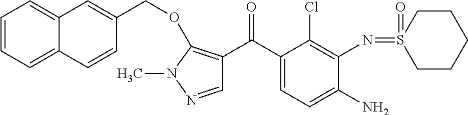 | amorphous ¹H-NMR (CDCl₃): 1.58 (m, 1H), 1.79 (m, 1H), 2.03 (m, 4H), 3.28 (m, 4H), 3.53 (s, 3H), 5.64 (s, 2H), 6.60 (d, 1H), 6.98 (d, 1H), 7.38 (s, 1H), 7.48-7.52 (m, 3H), 7.79-7.85 (m, 4H) |
| 20 | 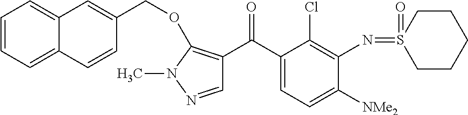 | amorphous ¹H-NMR (CDCl₃): 1.62 (m, 2H), 2.05 (m, 4H), 2.80 (s, 6H), 3.22 (m, 4H), 3.51 (s, 3H), 5.69 (s, 2H), 6.89 (d, 1H), 7.02 (d, 1H), 7.36 (s, 1H), 7.48-7.55 (m, 3H), 7.80-7.86 (m, 4H) |
| 21 | 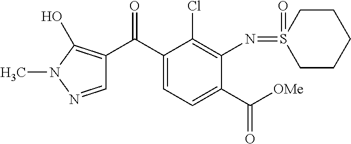 | m.p.: 204-206 |
| 22 | 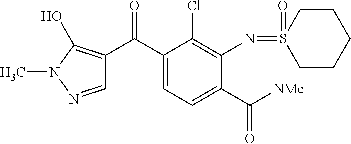 | amorphous ¹H-NMR (CDCl₃): 1.52 (m, 1H), 1.83 (m, 1H), 2.13 (m, 4H), 2.99 (d, 3H), 3.21 (m, 4H), 3.70 (s, 3H), 7.18 (d, 1H), 7.33 (s, 1H), 7.37 (br, 1H), 7.81 (d, 1H) |
| 23 | 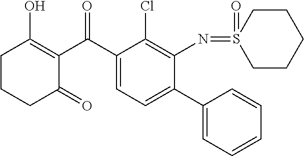 | m.p.: 166-168 |
| 24 | 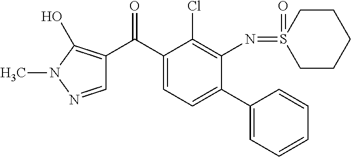 | amorphous ¹H-NMR (CDCl₃): 1.30 (m, 1H), 1.63 (m, 1H), 1.89 (m, 4H), 2.77 (m, 4H), 3.71 (s, 3H), 7.19 (d, 1H), 7.27-7.57(m, 7H) |
| 25 | 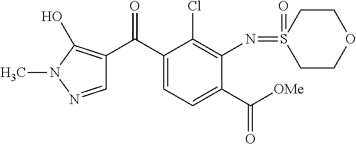 | m.p.: 147-149 |

TABLE 68

| Example | Structural formula | Physical property |
|---|---|---|
| 26 | 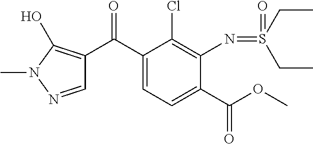 | m.p.: 69-73 |
| 27 | 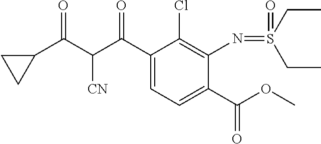 | amorphous 1H-NMR (CDCl3) 1.28 (m, 4H), 1.43 (t, 6H), 2.34 (m, 1H), 3.20 (q, 4H), 3.89 (s, 3H), 7.07 (d, 1H), 7.57 (d, 1H) |
| 28 | 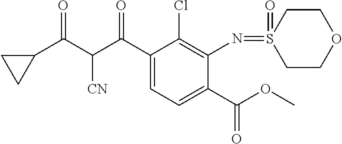 | amorphous 1H-NMR (CDCl3) 1.30 (m, 2H), 1.45 (m, 2H), 2.34 (m, 1H), 3.25 (m, 2H), 3.45 (m, 2H), 3.90 (s, 3H), 4.15 (m, 2H), 4.33 (m, 2H), 7.12 (d, 1H), 7.65 (d, 1H) |
| 29 | 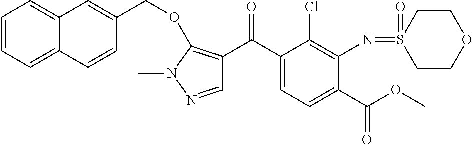 | amorphous 1H-NMR (CDCl3) 3.22 (m, 2H), 3.44 (m, 2H), 3.52 (s, 3H), 3.90 (s, 3H), 4.14 (m, 2H), 4.32 (m, 2H), 5.74 (s, 2H), 7.05 (d, 1H), 7.25 (s, 1H), 7.47-7.60 (m, 3H), 7.65 (d, 1H), 7.78-7.88 (m, 4H) |

TABLE 69

| Example | Structural formula | Physical property |
|---|---|---|
| 30 | 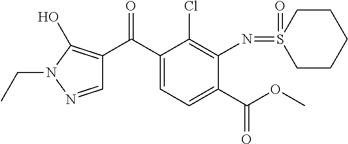 | m.p.: 201-203 |
| 31 | 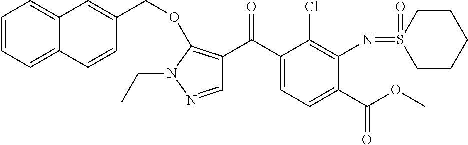 | amorphous 1H-NMR (CDCl3) 1.20 (t, 3H), 1.79 (m, 2H), 2.11 (m, 4H), 3.25 (m, 4H), 3.80-3.95 (m, 5H), 5.75 (s, 2H), 7.03 (d, 1H), 7.24 (s, 1H), 7.47-7.63 (m, 4H), 7.79-7.89 (m, 4H) |
| 32 | 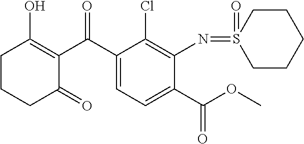 | amorphous 1H-NMR (CDCl3) 1.59 (m, 1H), 1.75 (m, 1H), 2.10 (m, 6H), 2.41 (t, 2H), 2.77 (t, 2H), 3.22 (m, 4H), 3.88 (s, 3H), 6.84 (d, 1H), 7.58 (d, 1H) |
| 33 | 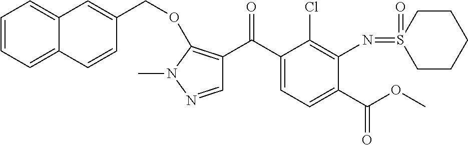 | amorphous 1H-NMR (CDCl3) 1.70 (m, 2H), 2.10 (m, 4H), 3.25 (m, 4H), 3.52 (s, 3H), 3.91 (s, 3H), 5.75 (s, 2H), 7.03 (d, 1H), 7.24 (s, 1H), 7.45-7.65 (m, 4H), 7.80-7.90 (m, 4H) |

TABLE 70

| | | |
|---|---|---|
| 34 | 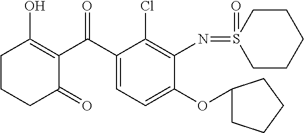 | amorphous 1H-NMR (CDCl3) 1.62 (m, 2H), 1.78-2.17 (m, 12H), 2.43 (t, 2H), 2.73 (t, 2H), 3.13 (m, 2H), 3.32 (m, 2H), 4.82 (m, 1H), 6.79 (d, 1H), 6.84 (d, 1H) |
| 35 | 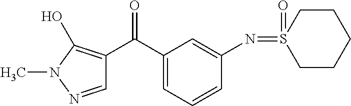 | m.p.: 112-113 |
| 36 | 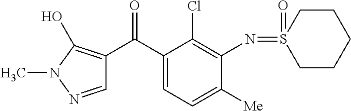 | amorphous ¹H-NMR (CDCl₃): 1.55 (m, 1H), 1.85 (m, 1H), 2.12 (m, 4H), 2.48 (s, 3H), 3.27 (m, 4H), 3.70 (s, 3H), 7.06 (d, 1H), 7.18 (d, 1H), 7.38 (s, 1H) |
| 37 | 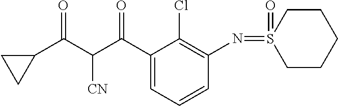 | amorphous ¹H-NMR (CDCl₃): 1.27 (m, 2H), 1.43 (m, 2H), 1.54 (m, 1H), 1.83 (m, 1H), 2.15 (m, 4H), 2.38 (m, 1H), 3.08 (m, 2H), 3.43 (m, 2H), 7.03 (d, 1H), 7.21 (t, 1H), 7.49 (d, 1H) |
| 38 | 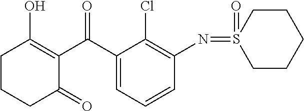 | amorphous ¹H-NMR (CDCl₃) 1.55 (m, 1H), 1.78 (m, 1H), 2.10 (m, 6H), 2.42 (t, 2H), 2.76 (t, 2H), 3.06 (m, 2H), 3.42 (m, 2H), 6.79 (d, 1H), 7.16 (t, 1H), 7.39 (d, 1H) |
| 39 | 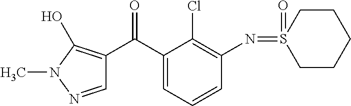 | m.p.: 177-179 |
| 40 | 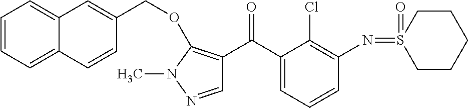 | amorphous ¹H-NMR (CDCl₃): 1.57 (m, 1H), 1.80 (m, 1H), 2.11 (m, 4H), 3.08 (m, 2H), 3.43 (m, 2H), 3.51 (s, 3H), 5.73 (s, 2H), 6.97 (d, 1H), 7.19 (t, 1H), 7.31 (s, 1H), 7.44-7.56 (m, 4H), 7.81-7.87 (m, 4H) |

TABLE 71

| | | |
|---|---|---|
| 41 | 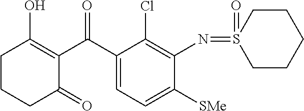 | m.p.: 178-180 |
| 42 | 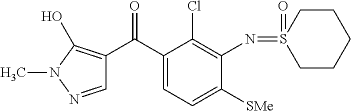 | m.p.: 157-159 |
| 43 | 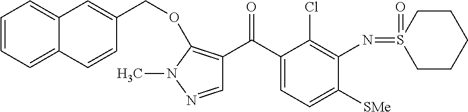 | amorphous ¹H-NMR (CDCl₃): 1.59 (m, 1H), 1.80 (m, 1H), 2.14 (m, 4H), 2.44 (s, 3H), 3.34 (m, 4H), 3.52 (s, 3H), 5.70 (s, 2H), 7.00 (d, 1H), 7.07 (d, 1H), 7.33 (s, 1H), 7.47-7.54 (m, 3H), 7.80-7.86 (m, 4H) |
| 44 | 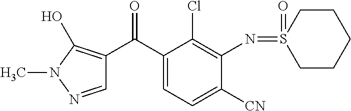 | m.p. 192-196 |
| 45 | 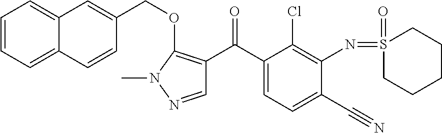 | amorphous ¹H-NMR (CDCl₃): 1.65 (m, 1H), 1.78 (m, 1H), 2.19 (m, 4H), 3.35 (m, 4H), 3.52 (s, 3H), 5.74 (s, 2H), 7.03 (d, 1H), 7.25 (s, 1H), 7.48-7.60 (m, 4H), 7.80-7.90 (m, 4H) |
| 46 | 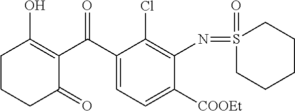 | amorphous ¹H-NMR (CDCl₃): 1.37 (t, 3H), 1.62 (m, 2H), 2.09 (m, 6H), 2.41 (t, 2H), 2.76 (t, 2H), 3.22 (m, 4H), 4.34 (q, 2H), 6.84 (d, 1H), 7.57 (d, 1H) |
| 47 | 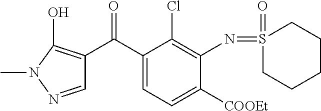 | m.p. 88-91 |
| 48 | 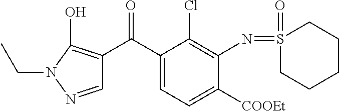 | amorphous ¹H-NMR (CDCl₃): 1.39 (t, 3H), 1.45 (t, 3H), 1.55-1.78 (m, 2H), 2.00-2.25 (m, 4H), 3.10-3.35 (m, 4H), 4.06 (q, 2H), 4.37 (q, 2H), 7.10 (d, 1H), 7.33 (s, 1H), 7.61 (d, 1H) |

TABLE 72

| | | |
|---|---|---|
| 49 | 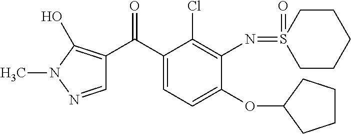 | m.p. 145-148 |
| 50 | 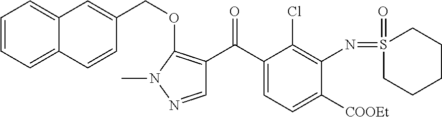 | viscous oil ¹H-NMR (CDCl₃): 1.40 (t, 3H), 1.67 (m, 2H), 2.15 (m, 4H), 3.24 (m, 4H), 3.51 (s, 3H), 4.37 (q, 2H), 5.75 (s, 2H), 7.02 (d, 1H), 7.25 (s, 1H), 7.45-7.65 (m, 4H), 7.80-7.89 (m, 4H) |
| 51 | 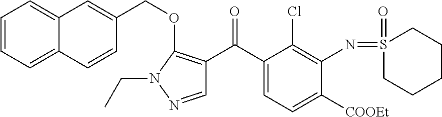 | viscous oil ¹H-NMR (CDCl₃): 1.20 (t, 3H), 1.40 (t, 3H), 1.55 (m, 2H), 2.15 (m, 4H), 3.25 (m, 4H), 3.89 (q, 2H), 4.37 (q, 2H), 5.75 (s, 2H), 7.03 (d, 1H), 7.26 (s, 1H), 7.47-7.63 (m, 4H), 7.80-7.89 (m, 4H) |
| 52 | 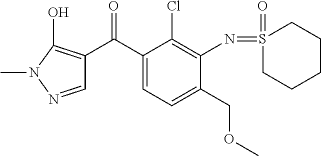 | m.p. 78-82 |

TABLE 73

| Example | Structural formula | Physical property |
|---|---|---|
| 53 | 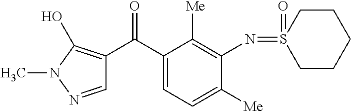 | 1H-NMR (CDCl3) 1.55 (m, 1H), 1.89 (m, 1H), 2.14 (m, 4H), 2.31 (s, 3H), 2.43 (s, 3H), 3.26 (m, 4H), 3.62 (s, 3H), 6.87 (d, 1H), 7.11 (d, 1H) |
| 54 | 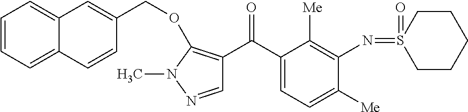 | 1H-NMR (CDCl3) 1.52 (m, 1H), 1.86 (m, 1H), 2.10 (m, 4H), 2.38 (s, 3H), 2.43 (s, 3H), 3.05 (m, 2H), 3.21 (m, 2H), 3.54 (s, 3H), 5.65 (s, 2H), 6.97 (d, 1H), 7.07 (d, 1H), 7.33 (s, 1H), 7.49-7.53 (m, 3H), 7.79-7.86 (m, 4H) |
| 55 | 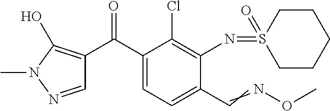 | m.p. 98-105 |
| 56 | 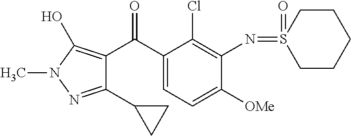 | m.p. 145-147 |

TABLE 74

| Example | Structural formula | Physical property |
|---|---|---|
| 57 | 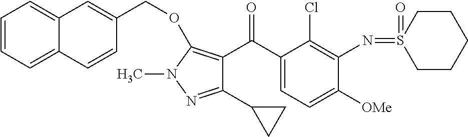 | 1H-NMR (CDCl3) 0.63 (m, 2H), 0.78 (m, 2H), 1.65 (m, 3H), 2.09 (m, 4H), 3.15 (m, 2H), 3.31 (m, 2H), 3.39 (s, 3H), 3.86 (s, 3H), 5.26 (s, 2H), 6.79 (d, 1H), 7.03 (d, 1H), 7.41 (dd, 1H), 7.48-7.51 (m, 2H), 7.70-7.84 (m, 4H) |
| 58 | 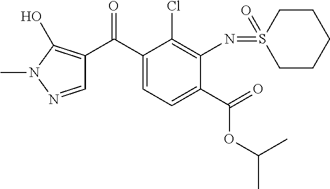 | 1H-NMR (CDCl3) 1.39 (d, 6H), 1.68 (m, 2H), 2.12 (m, 4H), 3.25 (m, 4H), 3.69 (s, 3H), 5.24 (m, 1H), 7.08 (d, 1H), 7.32 (s, 1H), 7.58 (d, 1H) |
| 59 | 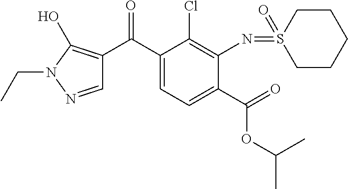 | 1H-NMR (CDCl3) 1.38 (d, 6H), 1.45 (t, 3H), 1.67 (m, 2H), 2.14 (m, 4H), 3.25 (m, 4H), 4.05 (q, 2H), 5.23 (m, 1H), 7.09 (d, 1H), 7.33 (s, 1H), 7.58 (d, 1H) |

TABLE 75

| Example | Structural formula | Physical property |
|---|---|---|
| 60 | 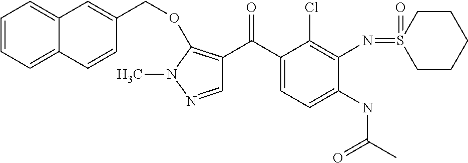 | 1H-NMR (CDCl3) 1.61 (m, 1H), 1.85 (m, 1H), 2.08 (m, 2H), 2.18 (m, 2H), 2.22 (s, 3H), 3.26 (m, 4H), 3.53 (s, 3H), 5.69 (s, 2H), 7.11 (d, 1H), 7.33 (s, 1H), 7.48-7.54 (m, 3H), 7.80-7.86 (m, 4H), 8.36 (d, 1H), 8.74(br, 1H) |
| 61 | 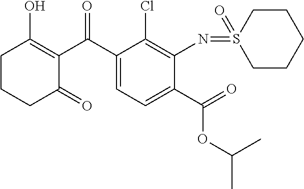 | 1H-NMR (CDCl3) 1.33 (d, 6H), 1.57 (m, 1H), 1.75 (m, 1H), 1.95-2.10 (m, 2H), 2.09 (m, 4H), 2.41 (t, 2H), 2.76 (t, 2H), 3.25 (m, 4H), 5.21 (m, 1H), 6.84 (d, 1H), 7.55 (d, 1H) |
| 62 | 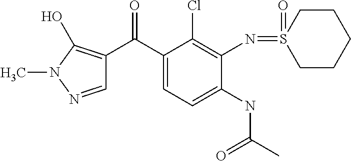 | 1H-NMR (CDCl3) 1.60 (m, 1H), 1.86 (m, 1H), 2.08 (m, 2H), 2.23 (m, 2H), 2.24 (s, 3H), 3.28 (m, 4H), 3.74 (s, 3H), 5.94 (br.s, 2H), 7.21 (d, 1H), 7.41 (s, 1H), 8.38 (d, 1H), 8.79 (br.s, 1H) |

TABLE 76

| Example | Structural formula | Physical property |
|---|---|---|
| 63 | 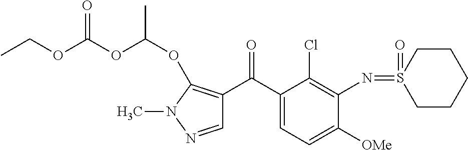 | 1H-NMR (CDCl3) 1.26 (t, 3H), 1.65 (m, 2H), 1.76 (d, 3H), 2.10 (m, 4H), 3.18 (m, 2H), 3.34 (m, 2H), 3.70 (s, 3H), 3.90 (s, 3H), 4.12 (q, 2H), 6.67 (q, 1H), 6.82 (d, 1H), 7.03 (d, 1H), 7.41 (s, 1H) |
| 64 | 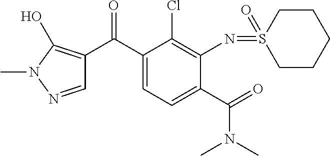 | 1H-NMR (CDCl3) 1.64 (m, 2H), 2.11 (m, 4H), 2.90 (s, 3H), 3.09 (s, 3H), 3.23 (m, 4H), 3.68 (s, 3H), 7.10 (d, 1H), 7.21 (d, 1H), 7.33 (s, 1H) |
| 65 | 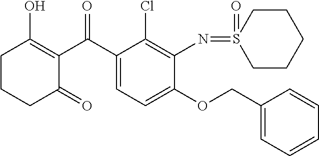 | m.p. 152-155 |
| 66 | 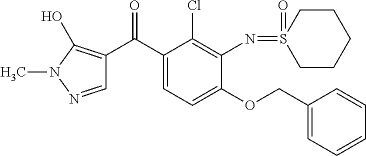 | m.p. 95-97 |

TABLE 77

| Example | Structural formula | Physical property |
|---|---|---|
| 67 | 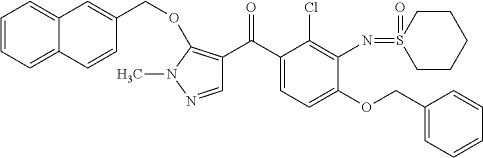 | 1H-NMR (CDCl3) 1.58 (m, 2H), 2.02 (m, 4H), 3.11 (m, 2H), 3.25 (m, 2H), 3.51 (s, 3H), 5.15 (s, 2H), 5.70 (s, 2H), 6.87 (d, 1H), 6.97 (d, 1H), 7.34-7.43 (m, 4H), 7.49-7.56 (m, 5H), 7.81-7.86 (m, 4H) |
| 68 | 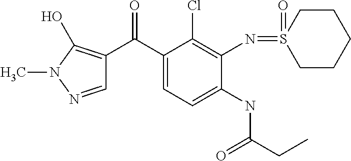 | 1H-NMR (CDCl3) 1.26 (t, 3H), 1.61 (m, 1H), 1.88 (m, 1H), 2.09 (m, 2H), 2.22 (m, 2H), 2.47 (q, 2H), 3.29 (m, 4H), 3.70 (s, 3H), 7.22 (d, 1H), 7.41 (s, 1H), 8.42 (d, 1H), 8.76 (br, 1H) |
| 69 | 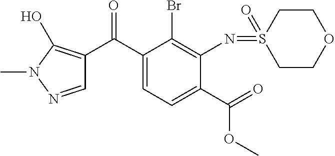 | m.p. 158-164 |
| 70 | 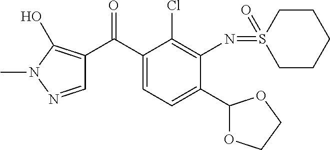 | 1H-NMR (CDCl3) 1.53 (m, 1H), 1.81 (m, 1H), 2.12 (m, 4H), 3.24 (m, 4H), 3.65 (s, 3H), 4.00-4.18 (m, 4H), 6.27 (s, 1H), 7.11 (d, 1H), 7.29 (s, 1H), 7.55 (d, 1H) |

TABLE 78

| Example | Structural formula | Physical property |
|---|---|---|
| 71 | 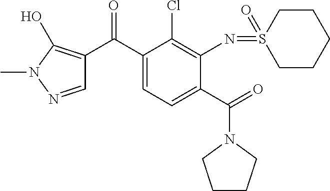 | 1H-NMR (CDCl3) 1.65 (m, 2H), 1.92 (m, 4H), 1.90-2.10 (m, 4H), 2.79-3.10 (m, 4H), 3.20-3.45 (m, 4H), 3.61 (m, 4H), 3.69 (s, 3H), 7.11 (d, 1H), 7.25 (s, 1H), 7.34 (d, 1H) |
| 72 | 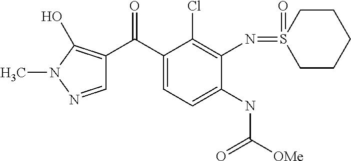 | 1H-NMR (CDCl3) 1.58 (m, 1H), 1.88 (m, 1H), 2.10-2.25 (m, 4H), 3.29 (m, 4H), 3.70 (s, 3H), 3.80 (s, 3H), 7.23 (d, 1H), 7.42 (s, 1H), 8.18 (d, 1H), 8.30 (br, 1H) |
| 73 | 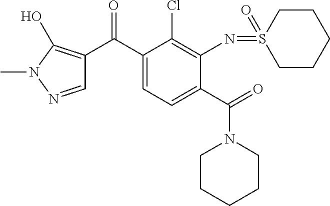 | 1H-NMR (CDCl3) 1.50-1.80 (m, 4H), 1.64 (m, 2H), 2.03 (m, 4H), 1.90-2.30 (m, 2H), 3.10-3.45 (m, 4H), 3.22 (m, 4H), 3.51 (br.d, 1H), 3.69 (s, 3H), 3.87 (br.d, 1H), 7.10 (d, 1H), 7.19 (d, 1H), 7.35 (s, 1H) |
| 74 | 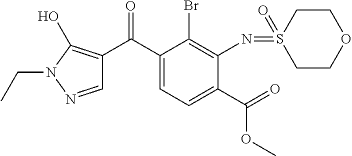 | m.p. 210-215 |

TABLE 79

| Example | Structural formula | Physical property |
|---|---|---|
| 75 | 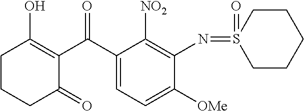 | 1H-NMR (CDCl3) 1.65 (m, 2H), 2.05 (m, 6H), 2.44 (t, 2H), 2.72 (t, 2H), 3.15 (m, 2H), 3.29 (m, 2H), 3.93 (s, 3H), 6.88 (d, 1H), 6.92 (d, 1H) |
| 76 | 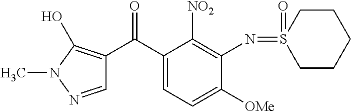 | m.p. 179-181 |
| 77 | 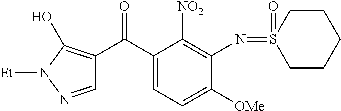 | m.p. 190-192 |
| 78 | 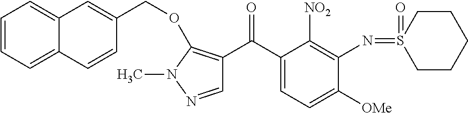 | 1H-NMR (CDCl3) 1.67 (m, 2H), 2.10 (m, 4H), 3.17 (m, 2H), 3.33 (m, 2H), 3.53 (s, 3H), 3.93 (s, 3H), 5.57 (s, 2H), 6.87 (d, 1H), 7.12 (d, 1H), 7.45-7.52 (m, 3H), 7.54 (s, 1H), 7.75-7.84 (m, 4H) |

TABLE 80

| Example | Structural formula | Physical property |
|---|---|---|
| 79 | 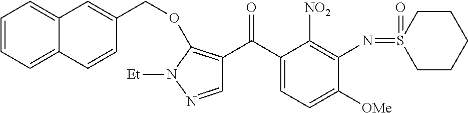 | 1H-NMR (CDCl3) 1.23 (t, 3H), 1.65 (m, 2H), 10 (m, 4H), 3.17 (m, 2H), 33 (m, 2H), 3.91 (q, 2H), 3.94 (s, 3H), 5.57 (s, 2H), 6.87 (d, 1H), 7.14 (d, 1H), 7.45-7.52 (m, 3H), 7.55 (s, 1H), 7.75-7.87 (m, 4H) |
| 80 | 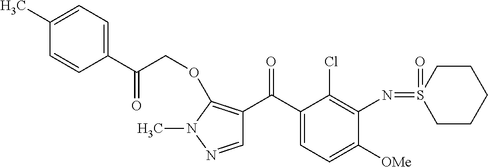 | 1H-NMR (CDCl3) 1.64 (m, 2H), 2.09 (m, H), 2.41 (s, 3H), 3.17 (m, 2H), 3.31 (m, 2H), 3.88 (s, 3H), 3.90 (s, 3H), 6.06 (s, 2H), 6.79 (d, 1H), 6.95 (d, 1H), 7.30 (s, 1H), 7.25-7.29 (m, 3H), 7.81 (d, 2H) |
| 81 | 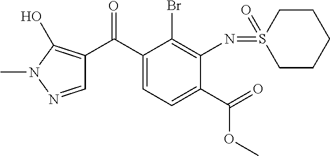 | 1H-NMR (CDCl3) 1.68 (m, 2H), 2.15 (m, 4H), 3.24 (m, 4H), 3.66 (s, 3H), 3.90 (s, 3H), 7.01 (d, 1H), 7.24 (s, 1H), 7.62 (d, 1H) |
| 82 | 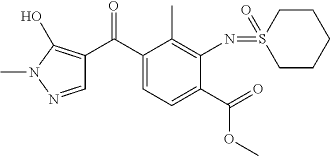 | 1H-NMR (CDCl3) 1.58 (m, 1H), 1.82 (m, 1H), 2.10 (m, 4H), 2.40 (s, 3H), 3.20 (m, 4H), 3.69 (s, 3H), 3.89 (s, 3H), 7.10 (d, 1H), 7.33 (s, 1H), 7.53 (d, 1H) |

TABLE 81

| Example | Structural formula | Physical property |
|---|---|---|
| 83 | 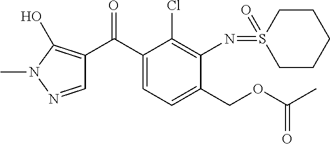 | m.p. 64-68 |
| 84 | 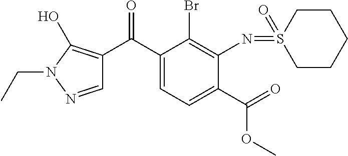 | 1H-NMR(CDCl3) 1.58(m, 1H), 1.72(m, 1H), 2.07(m, 4H), 2.17(br.s, 2H), 3.24(m, 4H), 3.88(s, 3H), 3.95(br.s, 3H), 6.95(d, 1H), 7.16(s, 1H), 7.58(d, 1H) |
| 85 | 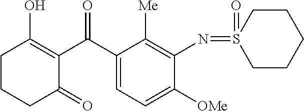 | 1H-NMR(CDCl3) 1.64(m, 2H), 2.05(m, 6H), 2.26(s, 3H), 2.44(t, 2H), 2.74(t, 2H), 3.18(m, 2H), 3.30(m, 2H), 3.85(s, 3H), 6.73(d, 1H), 6.83(d, 1H) |
| 86 | 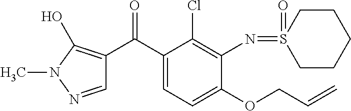 | 1H-NMR(CDCl3) 1.66(m, 2H), 2.15(m, 4H), 3.21(m, 2H), 3.36(m, 2H), 3.69(s, 3H), 4.64(d, 2H), 5.32(d, 1H), 5.45(d, 1H), 6.39(d, 1H), 6.83(d, 1H), 7.07(d, 1H), 7.43(s, 1H) |
| 87 | 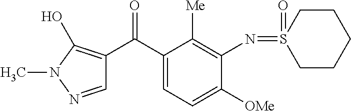 | 1H-NMR(CDCl3) 1.66(m, 2H), 2.11(m, 4H), 2.44(s, 3H), 3.19(m, 2H), 3.30(m, 2H), 3.69(s, 3H), 3.89(s, 3H), 6.78(d, 1H), 7.19(d, 1H), 7.45(s, 1H) |

TABLE 82

| Example | Structural formula | Physical property |
|---|---|---|
| 88 | 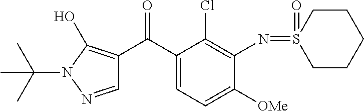 | m.p. 200-202 |
| 89 | 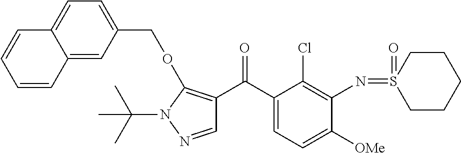 | 1H-NMR(CDCl3) 1.57(s, 9H), 1.61(m, 2H), 2.10(m, 4H), 3.20(m, 2H), 3.33(m, 2H), 3.90(s, 3H), 5.68(s, 2H), 6.82(d, 1H), 7.04(d, 1H), 7.32(s, 1H), 7.48-7.52(m, 2H), 7.65(dd, 1H), 7.84-7.95(m, 4H) |
| 90 | 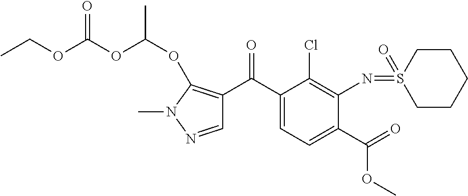 | 1H-NMR(CDCl3) 1.26(t, 3H), 1.61(m, 1H), 1.74(m, 1H), 1.79(d, 3H), 2.11(m, 4H), 3.24(m, 4H), 3.69(s, 3H), 3.90(s, 3H), 4.14(q, 2H), 6.70(q, 1H), 7.04(d, 1H), 7.30(s, 1H), 7.59(d, 1H) |
| 91 | 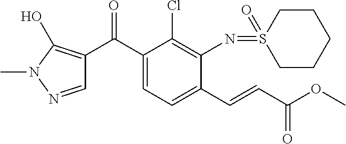 | m.p. 145-150 |

TABLE 83

| Example | Structural formula | Physical property |
|---|---|---|
| 92 | 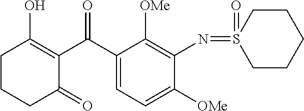 | m.p. 129-131 |
| 93 | 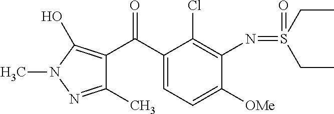 | 1H-NMR(CDCl3) 1.46(t, 6H), 1.80(s, 3H), 3.24(m, 4H), 3.62(s, 3H), 3.90(s, 3H), 6.84(d, 1H), 6.89(d, 1H) |
| 94 | 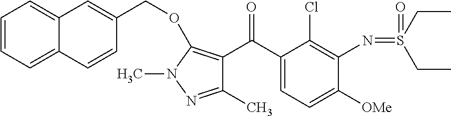 | 1H-NMR(CDCl3) 1.44(t, 6H), 2.03(s, 3H), 3.22(m, 4H), 3.41(s, 3H), 3.86(s, 3H), 5.24(s, 2H), 6.79(d, 1H), 6.98(d, 1H), 7.40(dd, 1H), 7.48-7.53 (m, 2H), 7.70(s, 1H), 7.80-7.84(m, 3H) |
| 95 | 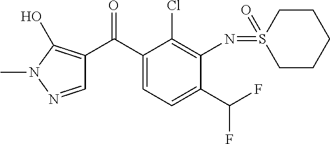 | 1H-NMR(CDCl3) 1.55(m, 1H), 1.83(m, 1H), 2.13(m, 4H), 3.24(m, 4H), 3.67(s, 3H), 7.10-7.25 (m, 1H), 7.17(t, 1H), 7.30 (s, 1H), 7.60(d, 1H) |

TABLE 84

| Example | Structural formula | Physical property |
|---|---|---|
| 96 | 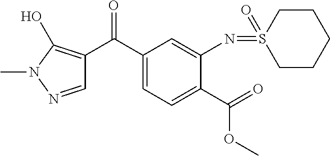 | m.p. 133-137 |
| 97 | 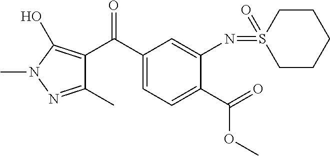 | 1H-NMR(CDCl3) 1.59(m, 2H), 1.77(m, 1H), 2.02(s, 3H), 2.04(m, 2H), 2.18(m, 2H), 3.07(m, 2H), 3.41(m, 2H), 3.62(s, 3H), 3.90(s, 3H), 7.13(dd, 1H), 7.51(s, 1H), 7.72(d, 1H) |
| 98 | 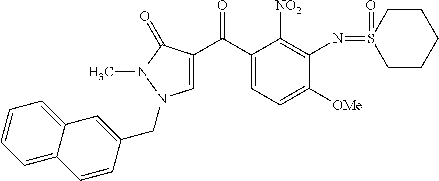 | 1H-NMR(CDCl3) 1.61(m, 2H), 2.04(m, 4H), 3.13(m, 2H), 3.26(m, 2H), 3.35(s, 3H), 3.92(s, 3H), 5.23(s, 2H), 6.92(d, 1H), 7.25(d, 1H), 7.55(m, 2H), 7.65(d, 2H), 7.82-7.90 (m, 3H), 8.03(s, 1H) |
| 99 | 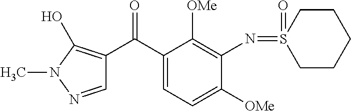 | m.p. 116-118 |

TABLE 85

| Example | Structural formula | Physical property |
|---|---|---|
| 100 | 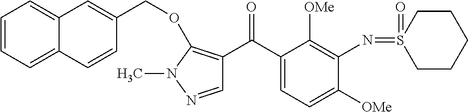 | 1H-NMR(CDCl3) 1.62(m, 2H), 2.06(m, 4H), 3.18(m, 2H), 3.33(m, 2H), 3.53(s, 3H), 3.78(s, 3H), 3.90(s, 3H), 5.65(s, 2H), 6.69(d, 1H), 7.06(d, 1H), 7.44(s, 1H), 7.48-7.53 (m, 3H), 7.80-7.86(m, 4H) |
| 101 | 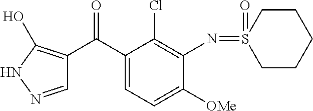 | 1H-NMR(DMSO) 1.57(m, 2H), 1.92(m, 4H), 3.20(m, 4H), 3.79(s, 3H), 6.91(d, 1H), 6.95(d, 1H), 7.59(s, 1H) |
| 102 | 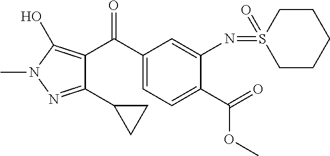 | 1H-NMR(CDCl3) 0.68-0.77(m, 2H), 0.85-0.93(m, 2H), 1.38-1.50(m, 1H), 1.58(m, 1H), 1.78(m, 1H), 2.04(m, 2H), 2.16(m, 2H), 3.07(m, 2H), 3.43(m, 2H), 3.58(s, 3H), 3.89(s, 3H), 7.29(dd, 1H), 7.66(d, 1H), 7.72(d, 1H) |

TABLE 86

| Example | Structural formula | Physical property |
|---|---|---|
| 103 | 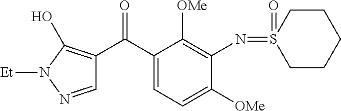 | 1H-NMR(CDCl3) 1.44(t, 3H), 1.68(m, 2H), 2.11(m, 4H), 3.26(m, 2H), 3.48(m, 2H), 3.81(s, 3H), 3.91(s, 3H), 4.05(q, 2H), 6.74(d, 1H), 7.27(d, 1H), 7.61(s, 1H) |
| 104 | 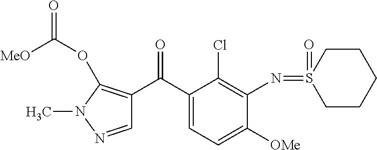 | 1H-NMR(CDCl3) 1.65(m, 2H), 2.11(m, 4H), 3.18(m, 2H), 3.33(m, 2H), 3.71(s, 3H), 3.90(s, 3H), 3.95(s, 3H), 6.82(d, 1H), 7.02(d, 1H), 7.59(s, 1H) |
| 105 | 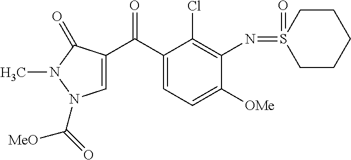 | 1H-NMR(CDCl3) 1.65(m, 2H), 2.11(m, 4H), 3.18(m, 2H), 3.32(m, 2H), 3.61(s, 3H), 3.90(s, 3H), 4.01(s, 3H), 6.83(d, 1H), 7.05(d, 1H), 8.27(s, 1H) |
| 106 | 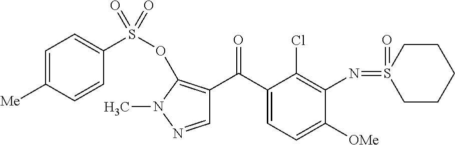 | 1H-NMR(CDCl3) 1.65(m, 2H), 2.10(m, 4H), 2.43(s, 3H), 3.18(m, 2H), 3.32(m, 2H), 3.84(s, 3H), 3.89(s, 3H), 6.75(d, 1H), 6.87(d, 1H), 7.34(d, 2H), 7.54(s, 1H), 7.82(d, 2H) |

TABLE 87

| Example | Structural formula | Physical property |
|---|---|---|
| 107 | 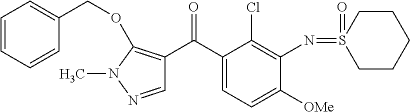 | 1H-NMR(CDCl3) 1.66(m, 2H), 2.09(m, 4H), 3.19(m, 2H), 3.34(m, 2H), 3.49(s, 3H), 3.91(s, 3H), 5.52(s, 2H), 6.83(d, 1H), 7.01(d, 1H), 7.34-7.41 (m, 6H) |
| 108 | 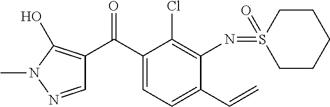 | 1H-NMR(CDCl3) 1.56(m, 1H), 1.89(m, 1H), 2.14(m, 4H), 3.25(m, 4H), 3.71(s, 3H), 5.38(dd, 1H), 5.74(dd, 1H), 7.13(d, 1H), 7.29(dd, 1H), 7.41(s, 1H), 7.54(d, 1H) |
| 109 | 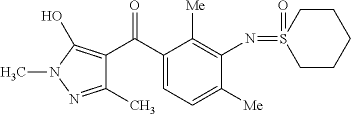 | m.p. 122-124 |
| 110 | 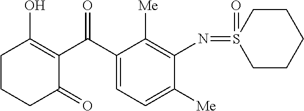 | 1H-NMR(CDCl3) 1.55(m, 1H), 1.86(m, 1H), 2.07(m, 6H), 2.25(s, 3H), 2.40(s, 3H), 2.42(t, 2H), 2.75(t, 2H), 3.23(m, 4H), 6.76(d, 1H), 7.05(d, 1H) |

TABLE 88

| Example | Structural formula | Physical property |
|---|---|---|
| 111 | 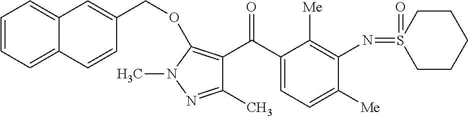 | 1H-NMR(CDCl3) 1.47(m, 1H), 1.84(m, 1H), 2.04(m, 4H), 2.07(s, 3H), 2.40(s, 3H), 2.42(s, 3H), 2.99(m, 2H), 3.13(m, 2H), 3.46(s, 3H), 5.12(s, 2H), 6.96(d, 1H), 7.08(d, 1H), 7.33(dd, 1H), 7.47-7.53 (m, 2H), 7.63(s, 1H), 7.78-7.84(m, 3H) |
| 112 | 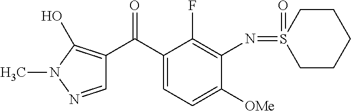 | m.p. 107-109 |
| 113 | 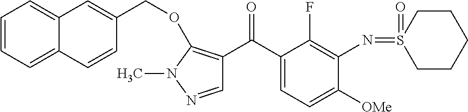 | 1H-NMR(CDCl3) 1.66( m, 2H), 2.12(m, 4H), 3.19(m, 2H), 3.30(m, 2H), 3.52(s, 3H), 3.92(s, 3H), 5.60(s, 2H), 6.73(dd, 1H), 7.21(t, 1H), 7.48-7.53 (m, 4H), 7.80-7.86(m, 4H) |
| 114 | 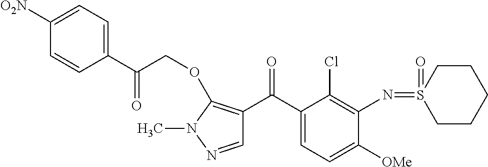 | 1H-NMR(CDCl3) 1.64(m, 2H), 2.09(m, 4H), 3.17(m, 2H), 3.31(m, 2H), 3.89(s, 6H), 6.13(s, 2H), 6.79(d, 1H), 6.94(d, 1H), 7.30(s, 1H), 8.10(d, 2H), 8.33(d, 2H) |

TABLE 89

| Example | Structural formula | Physical property |
|---|---|---|
| 115 | 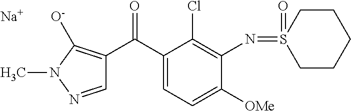 | m.p. 224-226 |
| 116 | 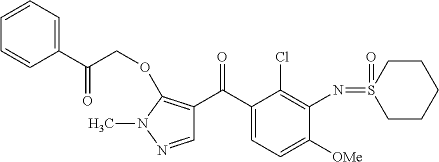 | 1H-NMR(CDCl3) 1.64(m, 2H), 2.09(m, 4H), 3.17(m, 2H), 3.32(m, 2H), 3.88(s, 3H), 3.90(s, 3H), 6.10(s, 2H), 6.79(d, 1H), 6.96(d, 1H), 7.30(s, 1H), 7.45-7.63(m, 3H), 7.90-7.93(m, 2H) |
| 117 | 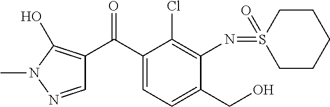 | 1H-NMR(CDCl3) 1.58(m, 1H), 1.88(m, 1H), 2.15(m, 4H), 3.32(m, 4H), 3.70(m, 2H), 4.67(s, 2H), 7.17(d, 1H), 7.35(s, 1H), 7.41(d, 1H) |
| 118 | 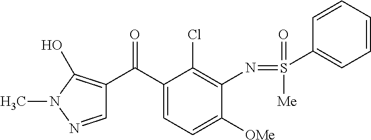 | 1H-NMR(CDCl3) 3.25(s, 3H), 3.63(s, 3H), 3.70(s, 3H), 6.75(d, 1H), 7.10(d, 1H), 7.43(s, 1H), 7.53-7.62(m, 3H), 8.08(dd, 2H) |

TABLE 90

| Example | Structural formula | Physical property |
|---|---|---|
| 119 | 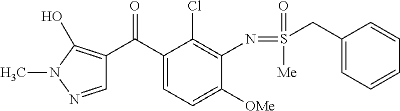 | 1H-NMR(CDCl3) 2.95(s, 3H), 3.70(s, 3H), 3.92(s, 3H), 4.55(m, 2H), 6.86(d, 1H), 7.15(d, 1H), 7.41(s, 1H), 7.38-7.50(m, 5H) |
| 120 | 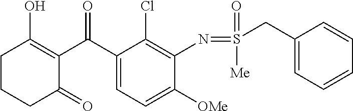 | 1H-NMR(CDCl3) 2.03(m, 2H), 2.43(t, 2H), 2.74(t, 2H), 2.88(s, 3H), 3.88(s, 3H), 4.51(s, 2H), 6.84(d, 1H), 6.91(d, 1H), 7.36-7.48(m, 5H) |
| 121 | 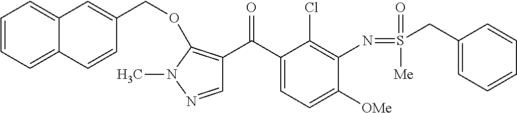 | 1H-NMR(CDCl3) 2.94(s, 3H), 3.52(s, 3H), 3.91(s, 3H), 4.55(m, 2H), 5.07(s, 2H), 6.84(d, 1H), 7.04(d, 1H), 7.35(s, 1H), 7.38-7.55(m, 7H), 7.80-7.86(m, 5H) |
| 122 | 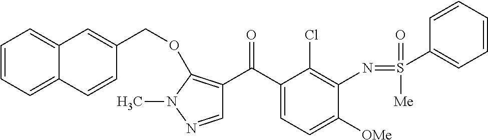 | 1H-NMR(CDCl3) 3.24(s, 3H), 3.52(s, 3H), 3.64(s, 3H), 5.70(s, 2H), 6.72(d, 1H), 6.98(d, 1H), 7.3 (s, 1H), 7.49-7.63(m, 6H), 7.81-7.87(m, 4H), 8.08-8.11(m, 2H) |

TABLE 91

| Example | Structural formula | Physical property |
|---|---|---|
| 123 | 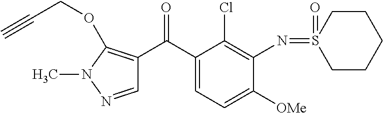 | m.p. 146-148 |
| 124 | 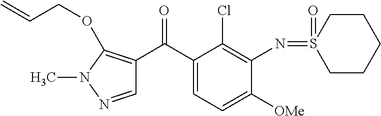 | 1H-NMR(CDCl3) 1.66(m, 2H), 2.09(m, 4H), 3.19(m, 2H), 3.38(m, 2H), 3.69(s, 3H), 3.90(s, 3H), 5.00(d, 2H), 5.27-5.43 (m, 2H), 5.97-6.06(m, 1H), 6.82(d, 1H), 7.00(d, 1H), 7.35(s, 1H) |
| 125 | 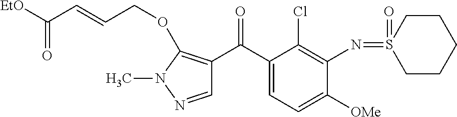 | 1H-NMR(CDCl3) 1.30(t, 3H), 1.65(m, 2H), 2.11(m, 4H), 3.18(m, 2H), 3.33(m, 2H), 3.72(s, 3H), 3.90(s, 3H), 4.22(q, 2H), 5.23(d, 2H), 6.15(d, 1H), 6.82(d, 1H), 6.98-7.04 (m, 2H), 7.33(s, 1H) |
| 126 | 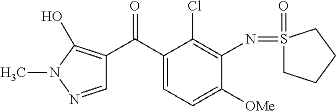 | m.p. 134-136 |

TABLE 92

| Example | Structural formula | Physical property |
|---|---|---|
| 127 | 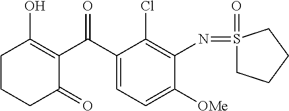 | m.p. 153-155 |
| 128 | 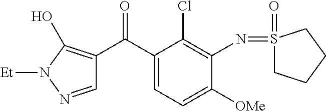 | m.p. 171-173 |
| 129 | 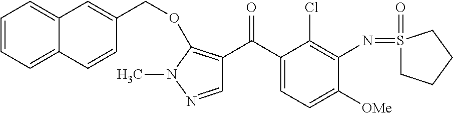 | 1H-NMR(CDCl3) 2.28(m, 4H), 3.24(m, 2H), 3.46(m, 2H), 3.51(s, 3H), 3.91(s, 3H), 5.70(s, 2H), 6.82(d, 1H), 6.99(d, 1H), 7.35(s, 1H), 7.48-7.55 (m, 3H), 7.80-7.86(m, 4H) |
| 130 | 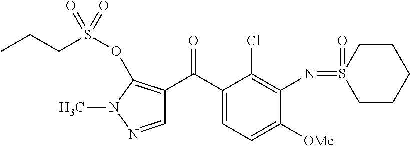 | 1H-NMR(CDCl3) 1.15(t, 3H), 1.66(m, 2H), 2.10(m, 6H), 3.19(m, 2H), 3.33(m, 2H), 3.66(m, 2H), 3.88(s, 3H), 3.91(s, 3H), 6.83(d, 1H), 7.03(d, 1H), 7.50 (s, 1H) |

TABLE 93

| Example | Structural formula | Physical property |
|---|---|---|
| 131 | 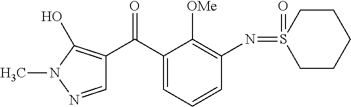 | m.p. 121-123 |
| 132 | 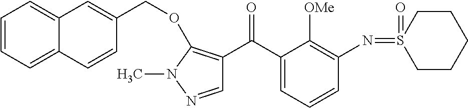 | 1H-NMR(CDCl3) 1.59(m, 1H), 1.75(m, 1H), 2.08(m, 4H), 3.11(m, 2H) 3.34(m, 2H), 3.52(s, 3H), 3.84(s, 3H), 5.69(s, 2H), 6.96(dd, 1H), 7.03(t, 1H), 7.35(dd, 1H), 7.39(s, 1H), 7.49-7.54(m, 3H), 7.81-7.86(m, 4H) |
| 133 | 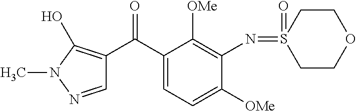 | m.p. 72-74 |
| 134 | 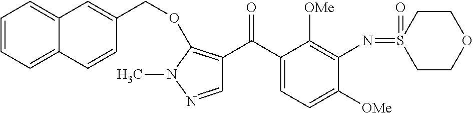 | 1H-NMR(CDCl3) 3.30(m, 2H), 3.44(m, 2H), 3.54(s, 3H), 3.77(s, 3H), 3.91(s, 3H), 4.13(m, 4H), 5.64(s, 2H), 6.70(d, 1H), 7.08(d, 1H), 7.43(s, 1H), 7.49-7.53(m, 3H), 7.80-7.86(m, 4H) |

TABLE 94

| Example | Structural formula | Physical property |
|---|---|---|
| 135 | 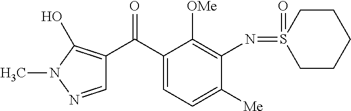 | m.p. 85-87 |
| 136 | 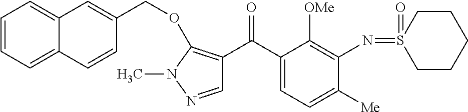 | 1H-NMR(CDCl3) 1.63(m, 2H), 2.04(m, 4H), 2.40(s, 3H), 3.20(m, 2H), 3.31(m, 2H), 3.54(s, 3H), 3.71(s, 3H), 5.67(s, 2H), 6.94(d, 1H), 7.00(d, 1H), 7.42(s, 1H), 7.47-7.54 (m, 3H), 7.81-7.87(m, 4H) |
| 137 | 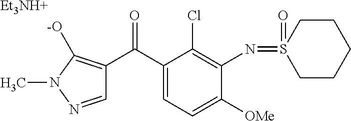 | 1H-NMR (CDCl3) 1.24(t, 9H), 1.64(m, 2H), 2.09(m, 4H), 3.06(q, 6H), 3.16(m, 2H), 3.32(m, 2H), 3.46(s, 3H), 3.87(s, 3H), 6.79(d, 1H), 6.97(d, 1H), 7.02(s, 1H) |
| 138 | 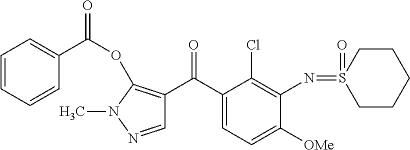 | 1H-NMR(CDCl3) 1.64(m, 2H), 2.08(m, 4H), 3.14(m, 2H), 3.29(m, 2H), 3.67(s, 3H), 3.74(s, 3H), 6.58(d, 1H), 6.93(d, 1H), 7.50(t, 2H), 7.65(m, 1H), 7.80(s, 1H), 8.03(dd, 2H) |

TABLE 95

| Example | Structural formula | Physical property |
|---|---|---|
| 139 | 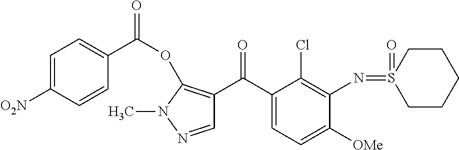 | m.p. 151-154 |
| 140 | 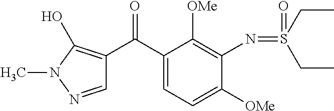 | m.p. 96-98 |
| 141 | 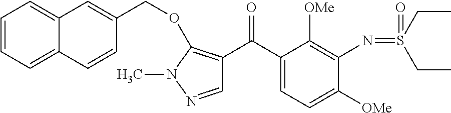 | 1H-NMR(CDCl3) 1.43(t, 6H), 3.22(m, 4H), 3.53(s, 3H), 3.78(s, 3H), 3.89(s, 3H), 5.66(s, 2H), 6.68(d, 1H), 7.06(d, 1H), 7.43(s, 1H), 7.48-7.54 (m, 3H), 7.80-7.86(m, 4H) |
| 142 | 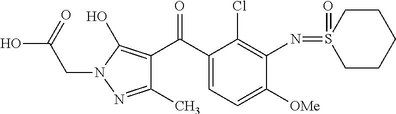 | 1H-NMR (CDCl3) 1.68(m, 2H), 1.83(s, 3H), 2.10(m, 4H), 3.20(m, 2H), 3.36(m, 2H), 3.90(s, 3H), 4.79(s, 2H), 6.86(d, 1H), 6.92(d, 1H) |

TABLE 96

| Example | Structural formula | Physical property |
|---|---|---|
| 143 | 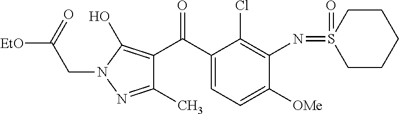 | 1H-NMR (CDCl3) 1.30 (t, 3H), 1.66 (m, 2H), 1.82 (s, 3H), 2.10 (m, 4H), 3.19 (m, 2H), 3.33 (m, 2H), 3.90 (s, 3H), 4.25 (q, 2H), 4.70 (s, 2H), 6.85 (d, 1H), 6.92 (d, 1H) |
| 144 | 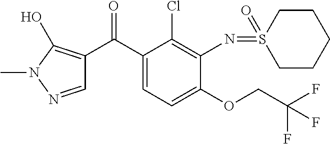 | m.p. 97-99 |
| 145 | 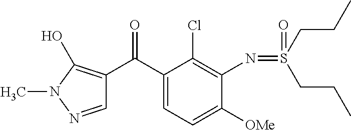 | 1H-NMR (CDCl3) 1.08 (t, 6H), 1.98 (m, 4H), 3.18 (m, 4H), 3.69 (s, 3H), 3.91 (s, 3H), 6.84 (d, 1H), 7.12 (d, 1H), 7.42 (s, 1H) |
| 146 | 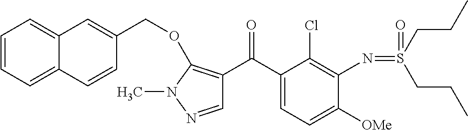 | 1H-NMR (CDCl3) 1.07 (t, 6H), 1.96 (m, 4H), 3.18 (m, 4H), 3.51 (s, 3H), 3.90 (s, 3H), 5.70 (s, 2H), 6.81 (d, 1H), 7.01 (d, 1H), 7.33 (s, 1H), 7.48-7.55 (m, 3H), 7.80-7.86 (m, 4H) |

TABLE 97

| Example | Structural formula | Physical property |
|---|---|---|
| 147 | 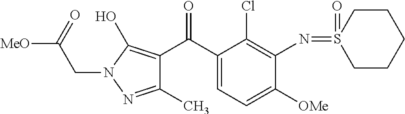 | 1H-NMR (CDCl3) 1.66 (m, 2H), 1.82 (s, 3H), 2.12 (m, 4H), 3.19 (m, 2H), 3.34 (m, 2H), 3.80 (s, 3H), 3.91 (s, 3H) 4.72 (s, 2H), 6.85 (d, 1H), 6.93 (d, 1H) |
| 148 | 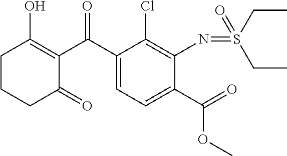 | 1H-NMR (CDCl3) 1.42 (t, 6H), 2.00-2.10 (m, 2H), 2.41 (t, 2H), 2.76 (t, 2H), 3.18 (m, 4H), 3.87 (s, 3H), 6.84 (d, 1H), 7.55 (d, 1H) |
| 149 | 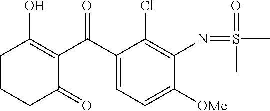 | m.p. 150-152 |
| 150 | 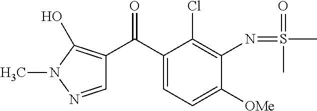 | m.p. 164-166 |

TABLE 98

| Example | Structural formula | Physical property |
|---|---|---|
| 151 | 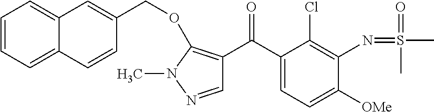 | 1H-NMR (CDCl3) 3.21 (s, 6H), 3.51 (s, 3H), 3.91 (s, 3H), 5.69 (s, 2H), 6.83 (d, 1H), 7.06 (d, 1H), 7.34 (s, 1H), 7.48-7.55 (m, 3H), 7,80-7.86 (m, 4H) |
| 152 | 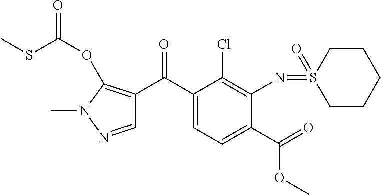 | 1 H-NMR(CDC13) 1.67 (m, 2H), 2.13 (m, 4H), 2.50 (s, 3H), 3.22 (m,4H), 3.72 (s, 3H), 3.89 (s, 3H), 6.98 (d, 1H), 7.56 (d, 1H), 7.65 (s, 1H) |
| 153 | 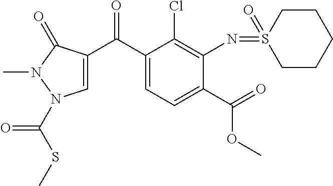 | 1H-NMR (CDCl3) 1.61 (m, 1H), 1.75 (m, 1H), 2.12 (m,4H), 2.55 (s, 3H), 3.24 (m, 4H), 3.60 (s, 3H), 3.89 (s, 3H), 7.01 (d, 1H), 7.58 (d, 1H), 8.35 (s, 1H) |
| 154 | 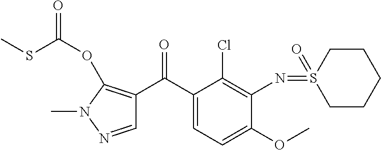 | m.p. 163-165 |

TABLE 99

| Example | Structural formula | Physical property |
|---|---|---|
| 155 | 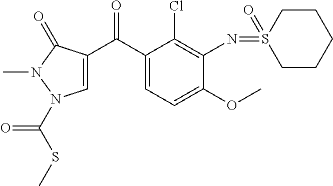 | 1H-NMR (CDCl3) 1.64 (m, 2H), 2.10 (m, 4H), 2.54 (s, 3H), 3.17 (m, 2H), 3.32 (m, 2H), 3.61 (s, 3H), 3.89 (s, 3H), 6.83 (d, 1H), 7.07 (d, 1H), 8.36 (s, 1H) |
| 156 | 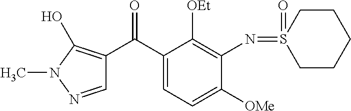 | m.p. 111-113 |
| 157 | 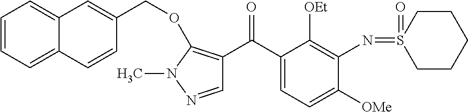 | 1H-NMR (CDCl3) 1.17 (t, 3H), 1.61 (m, 2H), 2.11 (m, 4H), 3.18 (m, 2H), 3.31 (m, 2H), 3.54 (s, 3H), 3.90 (s, 3H), 4.03 (q, 2H), 5.63 (s, 2H), 6.98 (d, 1H), 7.06 (d, 1H), 7.45 (s, 1H), 7.47-7.53 (m, 3H), 7.80-7.86 (m, 4H) |
| 158 | 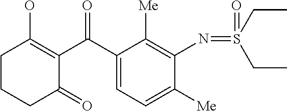 | 1H-NMR (CDCl3) 1.42 (t, 6H), 2.06 (m, 2H), 2.24 (s, 3H), 2.37 (s, 3H), 2.42 (t, 2H), 2.74 (t, 2H), 3.03-3.22 (m, 4H), 6.71 (d, 1H), 7.02 (d, 1H) |

TABLE 100

| Example | Structural formula | Physical property |
|---|---|---|
| 159 | 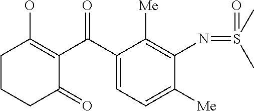 | 1H-NMR (CDC13) 2.02 (m, 2H), 2.18 (s, 3H), 2.35 (s, 3H), 2.41 (t 2H), 2.75 (t, 2H), 3,09 (s, 6H), 6.74 (d, 1H), 7.05 (d, 1H) |
| 160 | 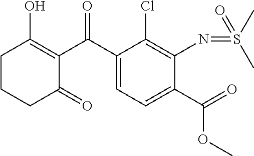 | m.p. 135-137 |
| 161 | 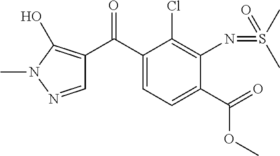 | m.p. 115-125 |
| 162 | 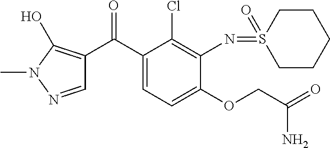 | m.p. 230up |

TABLE 101

| Example | Structural formula | Physical property |
|---|---|---|
| 163 | 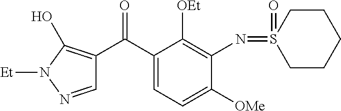 | m.p. 124-126 |
| 164 | 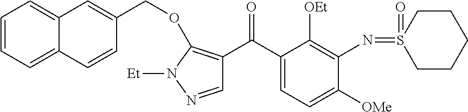 | 1H-NMR (CDCl3) 1.17 (t 3H), 1.24 (t, 3H), 1.61 (m, 2H), 2.06 (m, 4H), 3.18 (m, 2H), 3.31 (m, 2H), 3.90 (s, 3H), 3.91 (q, 2H), 4.03 (q, 2H), 5.63 (s, 2H), 6.68 (d, 1H), 7.08 (d, 1H), 7.47 (s, 1H), 7.48-7.54 (m, 3H), 7.80-7.86 (m, 4H) |
| 165 | 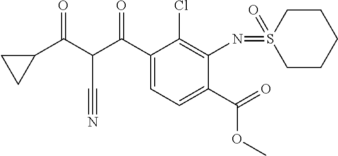 | 1H-NMR (CDCl3) 1.20-1.34 (m, 2H), 1.35-1.50 (m, 2H), 1.62 (m, 1H), 1.74 (m, 1H), 2.13 (m, 4H), 2.30-2.40 (m, 1H), 3.21 (m, 4H), 3.90 (s, 3H), 7.09 (d, 1H), 7.61 (d, 1H) |
| 166 | 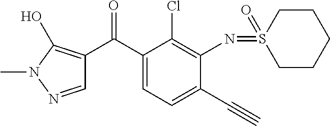 | 1H-NMR (CDCl3) 1.58 (m, 1H), 1.80 (m, 1H), 2.15 (m, 4H), 3.33 (m, 4H), 3.64 (s, 1H), 3.69 (s, 3H), 7.04 (d, 1H), 7.35 (s, 1H), 7.45 (d, 1H) |

TABLE 102

| Example | Structural formula | Physical property |
|---|---|---|
| 167 | 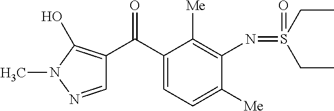 | m.p. 126-128 |
| 168 | 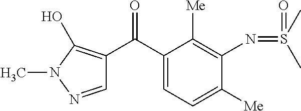 | m.p. 133-135 |
| 169 | 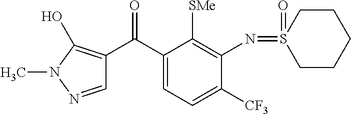 | m.p. 155-157 |
| 170 | 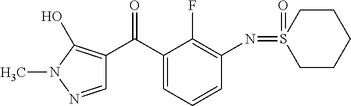 | m.p. 162-164 |
| 171 | 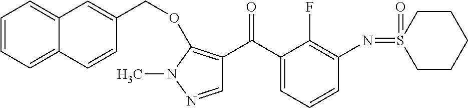 | 1H-NMR (CDCl3) 1.60 (m, 1H), 1.75 (m, 1H), 2.09 (m, 4H), 3.11 (m, 2H), 3.35 (m, 2H), 3.52 (s, 3H), 5.64 (s, 2H), 7.05-7.14 (m, 2H), 7.39-7.54 (m, 5H), 7.82-7.87 (m, 4H) |

TABLE 103

| Example | Structural formula | Physical property |
|---|---|---|
| 172 | 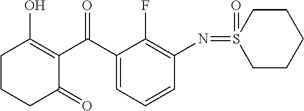 | 1H-NMR (CDCl3) 1.58 (m, 1H), 1.74 (m, 1H), 2.09 (m, 6H), 2.46 (t, 2H), 2.74 (t, 2H), 3.10 (m, 2H), 3.36 (m, 2H), 6.99-7.08 (m, 2H), 7.37 (td, 1H) |
| 173 | 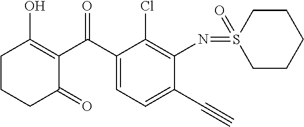 | 1H-NMR (CDCl3) 1.55 (m,1H), 1.76 (m, 1H), 2.04 (t, 2H), 2.11 (m, 4H), 2.41 (t, 2H), 2.75 (t, 2H), 3.28 (m, 4H), 3.30(s, 1H), 6.79 (d, 1H), 7.41 (d, 1H) |
| 174 | 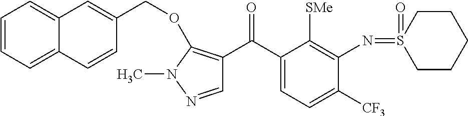 | 1H-NMR (CDCl3) 1.52 (m, 1H), 1.80 (m, 1H), 2.04 (s, 3H), 2.14 (m, 4H), 3.25 (m, 2H), 3.40 (m, 2H), 3.49 (s, 3H), 5.76 (s, 2H), 6.98 (d, 1H), 7.20 (s, 1H), 7.48-7.56 (m, 3H), 7.62 (d, 1H), 7.79-7.87 (m, 4H) |
| 175 | 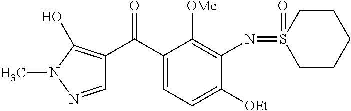 | m.p. 109-111 |

TABLE 104

| Example | Structural formula | Physical property |
|---|---|---|
| 176 | 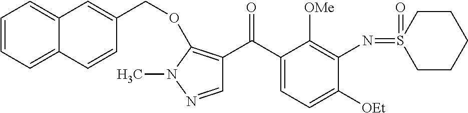 | 1H-NMR (CDCl3) 1.49 (t, 3H), 1.63 (m, 2H), 2.08 (m, 4H), 3.16 (m, 2H), 3.34 (m, 2H), 3.53 (s, 3H), 3.79 (s, 3H), 4.12 (q, 2H), 5.64 (s, 2H), 6.67 (d, 1H), 7.03 (d, 1H), 7.44 (s, 1H), 7.48-7.54 (m, 3H), 7.80-7.86 (m, 4H) |
| 177 | 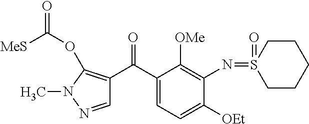 | 1H-NMR (CDCl3) 1.48 (t, 3H), 1.64 (m, 2H), 2.09 (m, 4H), 2.44 (s, 3H), 3.17 (m, 2H), 3.34 (m, 2H), 3.72 (s, 3H), 3.75 (s, 3H), 4.10 (q, 2H), 6.66 (d, 1H), 7.05 (d, 1H), 7.69 (s, 1H) |
| 178 | 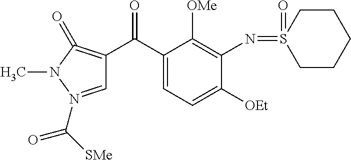 | 1H-NMR (CDCl3) 1.48 (t, 3H), 1.67 (m, 2H), 2.11 (m, 4H), 2.55 (s, 3H), 3.09 (m, 2H), 3.56 (m, 2H), 3.59 (s, 3H), 3.68 (s, 3H), 4.12 (q, 2H), 6.69 (d, 1H), 7.16 (d, 1H), 8.40 (s, 1H) |
| 179 | 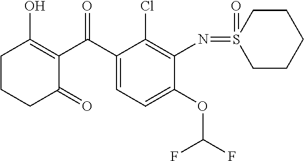 | m.p. 125-127 |

TABLE 105

| Example | Structural formula | Physical property |
|---|---|---|
| 180 | 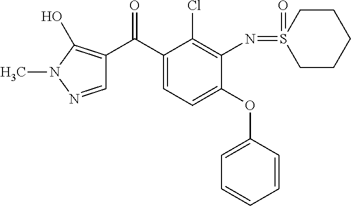 | m.p. 134-137 |
| 181 | 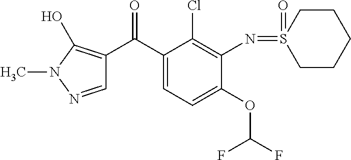 | m.p. 158-161 |
| 182 | 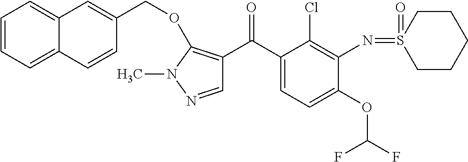 | 1H-NMR (CDCl3) 1.67 (m, 2H), 2.14 (m, 4H), 3.22 (m, 2H), 3.34 (m, 2H), 3.52 (s, 3H), 5.73 (s, 2H), 6.59 (t, 1H), 6.99 (d, 1H), 7.16 (d, 1H), 7.31 (s, 1H), 7.48-7.55 (m, 3H), 7.81-7.87 (m, 4H) |
| 183 | 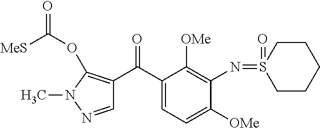 | 1H-NMR (CDCl3) 1.64 (m, 2H), 2.09 (m, 4H), 2.44 (s, 3H), 3.20 (m, 2H), 3.33 (m, 2H), 3.74 (s, 3H), 3.77 (s, 3H), 3.89 (s, 3H), 6.68 (d, 1H), 7.08 (d, 1H), 7.69 (s, 1H) |

TABLE 106

| Example | Structural formula | Physical property |
|---|---|---|
| 184 | 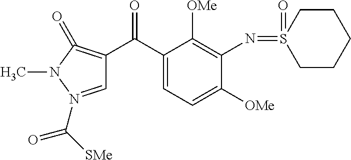 | 1H-NMR (CDCl3) 1.58 (m, 2H), 2.04 (m, 4H), 2.55 (s, 3H), 3.12 (m, 2H), 3.49 (m, 2H), 3.59 (s, 3H), 3.68 (s, 3H), 3.89 (s, 3H), 6.72 (d, 1H), 7.18 (d, 1H), 8.40 (s, 1H) |
| 185 | 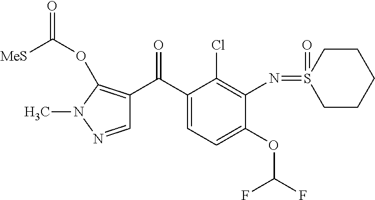 | 1H-NMR (CDCl3) 1.68 (m, 2H), 2.14 (m, 4H), 2.43 (s, 3H), 3.21 (m, 2H), 3.34 (m, 2H), 3.72 (s, 3H), 6.60 (t, 1H), 6.95 (d, 1H), 7.14 (d, 1H), 7.73 (s, 1H) |
| 186 | 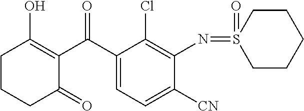 | m.p. 124-126 |
| 187 | 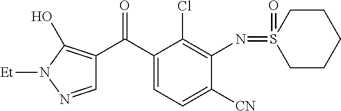 | m.p. 159-161 |

TABLE 107

| Example | Structural formula | Physical property |
|---|---|---|
| 188 | 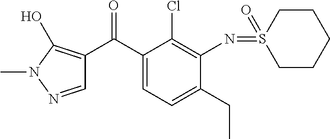 | 1H-NMR (CDCl3) 1.24 (t, 3H), 1.53 (m, 1H), 1.85 (m, 1H), 2.11 (m, 4H), 2.91 (q, 2H), 3.27 (m, 4H), 3.69 (s, 3H), 7.10 (d, 1H), 7.21 (d, 1H), 7.84 (s, 1H) |
| 189 | 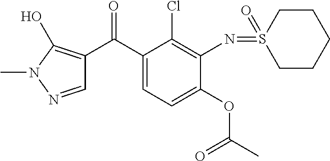 | m.p. 135-139 |
| 190 | 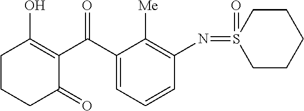 | 1H-NMR (CDCl3) 1.59 (m, 1H), 1.77 (m, 1H), 2.03 (m, 6H), 2.20 (s, 3H), 2.43 (t, 2H), 2.76 (t, 2H), 3.11 (m, 2H), 3.48 (m, 2H), 6.71 (d, 1H), 7.08 (t, 1H), 7.29 (d, 1H) |
| 191 | 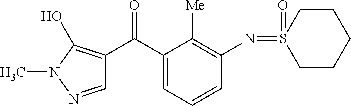 | m.p. 166-168 |

TABLE 108

| Example | Structural formula | Physical property |
|---|---|---|
| 192 | 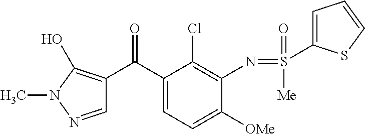 | m.p. 90-92 |
| 193 | 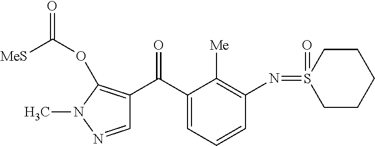 | m.p. 149-151 |
| 194 | 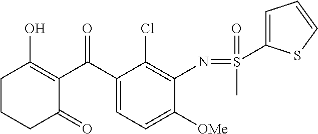 | m.p. 159-162 |
| 195 | 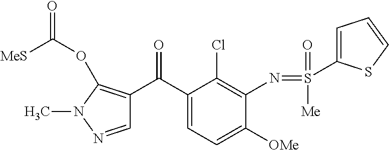 | 1H-NMR (CDCl3) 2.43 (s, 3H), 3.35 (s, 3H), 3.74 (s, 3H), 3.77 (s, 3H), 6.77 (d, 1H), 7.02 (d, 1H), 7.12 (dd, 1H), 7.65 (dd, 1H), 7.66 (s, 1H), 7.72 (dd, 1H) |

TABLE 109

| Example | Structural formula | Physical property |
|---|---|---|
| 196 | 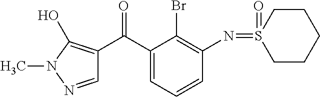 | m.p. 177-179 |
| 197 | 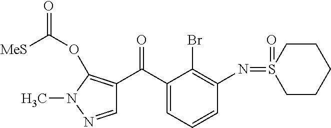 | 1H-NMR (CDCl3) 1.56 (m, 1H), 1.83 (m, 1H), 2.02 (m, 2H), 2.21 (m, 2H), 2.41 (s, 3H), 3.06 (m, 2H), 3.45 (m, 2H), 3.72 (s, 3H), 6.87 (d, 1H), 7.20 (t, 1H), 7.42 (d, 1H), 7.68 (s, 1H) |
| 198 | 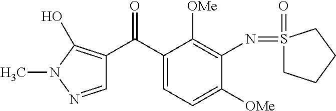 | m.p. 151-153 |
| 199 | 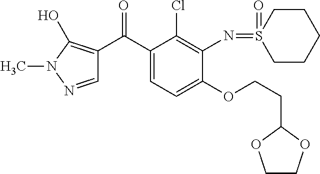 | m.p. 99-101 |

| Example | Structural formula | Physical property |
|---|---|---|
| 200 | 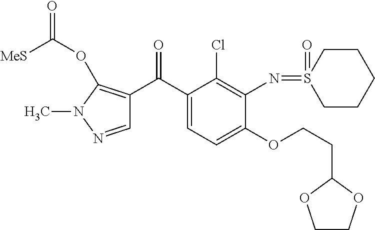 | m.p. 159-161 |
| 201 | 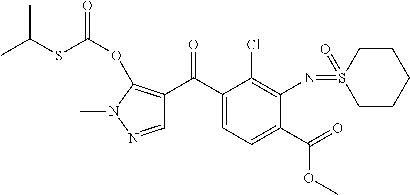 | 1H-NMR (CDCl3) 1.39 (d, 6H), 1.62 (m, 1H), 1.73 (m, 1H), 2.07 (m, 2H), 2.19 (m, 2H), 3.15 (m, 2H), 3.27 (m, 2H), 3.45-3.65 (m, 1H), 3.72 (s, 3H), 3.89 (s, 3H), 6.99 (d, 1H), 7.57 (d, 1H), 7.65 (s, 1H) |
| 202 | 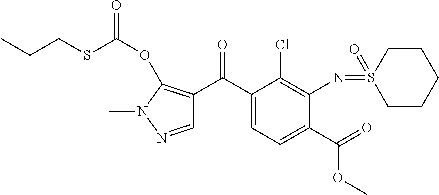 | 1H-NMR (CDCl3) 1.02 (t, 3H), 1.67 (m, 2H), 1.65-1.80 (m, 2H),2.10 (m, 2H), 2.20 (m, 2H), 2.90 (t, 2H), 3.15 (m, 2H), 3.27 (m, 2H), 3.72 (s, 3H), 3.89 (s, 3H), 6.99 (d, 1H), 7.57 (d, 1H), 7.63 (s, 1H) |

TABLE 111

| Example | Structural formula | Physical property |
|---|---|---|
| 203 | 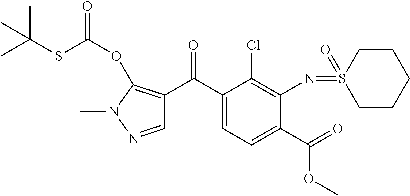 | 1H-NMR (CDCl3) 1.50 (s, 9H), 1.61 (m, 1H), l.71 (m, 1H), 2.09 (m, 2H), 2.20 (m, 2H), 3.15 (m, 2H), 3.27 (ddd, 2H), 3.71 (s, 3H), 3.89 (s, 3H), 6.98 (d, 1H), 7.56 (d, 1H), 7.67 (s, 1H) |
| 204 | 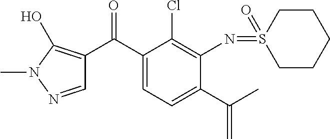 | 1H-NMR (CDCl3) 1.52 (m, 1H), 1.77 (m, 1H), 2.11 (m, 4H), 2.17 (s, 3H), 3.19 (m, 4H), 3.69 (s, 3H), 5.08 (s, 1H), 5.18-5.20 (m, 1H), 7.07 (d, 1H), 7.17 (d, 1H), 7.41 (s, 1H) |
| 205 | 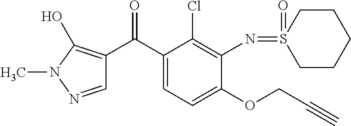 | m.p. 169-171 |
| 206 | 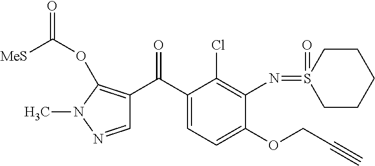 | 1H-NMR (CDCl3) 1.64 (m, 2H), 2.09 (m, 4H), 2.43 (s, 3H), 2.55 (t, 1H), 3.20 (m, 2H), 3.37 (m, 2H), 3.73 (s, 3H), 4.77 (d, 2H), 6.98 (m, 2H), 7.67 (s, 1H) |

TABLE 112

| Example | Structural formula | Physical property |
|---|---|---|
| 207 | 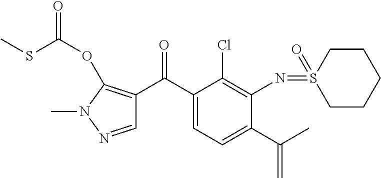 | 1H-NMR (CDCl3) 1.52 (m, 1H), 1.76 (m, 1H), 2.07 (m, 4H), 2.16 (dd, 3H), 2.41 (s, 3H), 3.17 (m, 4H), 3.73 (s, 3H), 5.00-5.07 (m, 1H), 5.15-5.20 (m, 1H), 6.96 (d, 1H), 7.13 (d, 1H), 7.70 (s, 1H) |
| 208 | 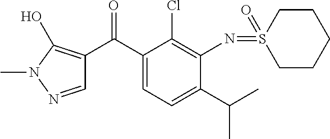 | m.p. 102-104 |
| 209 | 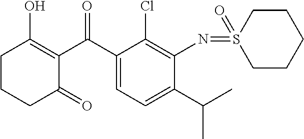 | 1H-NMR (CDCl3) 1.20 (d, 6H), 1.50 (m, 1H), 1.80 (m, 1H), 1.95-2.10 (m, 2H), 2.10 (m, 4H), 2.42 (t, 2H), 2.75 (t, 2H), 3.23 (m, 4H), 3.65-3.80 (m, 1H), 6.88 (d, 1H), 7.21 (d, 1H) |
| 210 | 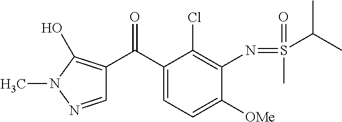 | m.p. 167-169 |

TABLE 113

| Example | Structural formula | Physical property |
|---|---|---|
| 211 | 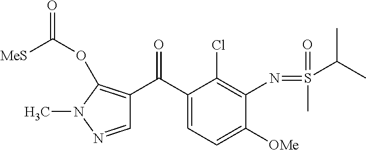 | 1H-NMR (CDCl3) 1.55 (d, 6H), 2.43 (s, 3H), 2.97 (s, 3H), 3.39 (m, 1H), 3.73 (s, 3H), 3.91 (s, 3H), 6.82 (d, 1H), 7.01 (d, 1H), 7,64 (s, 1H) |
| 212 | 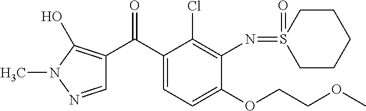 | m.p. 153-155 |
| 213 | 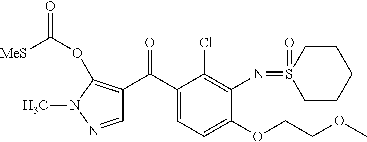 | 1H-NMR (CDCl3) 1.66 (m, 2H), 2.10 (m, 4H), 2.43 (s, 3H), 3.22 (m, 2H), 3.40 (m, 2H), 3.43 (s, 3H), 3.73 (s, 3H), 3.86 (m, 2H), 4.18 (m, 2H), 6.80 (d, 1H), 6.95 (d, H), 7.64 (s, 1H) |
| 214 | 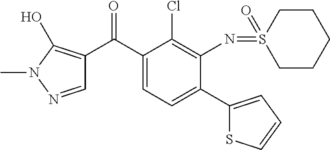 | m.p. 158-164 |
| 215 | 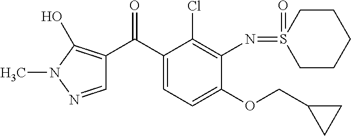 | m.p. 188-190 |

TABLE 114

| Example | Structural formula | Physical property |
|---|---|---|
| 216 | 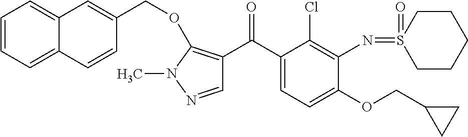 | 1H-NMR (CDCl3) 0.38 (m, 2H), 0.65 (m, 2H), 1.38 (m, 1H), 1.66 (m, 2H), 2.11 (m, 4H), 3.28 (m, 2H), 3.43 (m, 2H), 3.51 (s, 3H), 3.88 (d, 2H), 5.69 (s, 2H), 6.96 (d, 1H), 7.07 (d, 1H), 7.35 (s, 1H), 7.48-7.55 (m, 3H), 7.80-7.86 (m, 4H) |
| 217 | 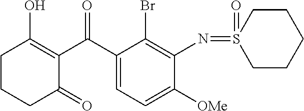 | 1H-NMR(CDC13) 1.64 (m, 2H), 2.06 (m, 6H), 2.43 (t, 2H), 2.74 (t, 2H), 3.17 (m, 2H), 3.34 (m, 2H), 3.88 (s, 3H), 6.79 (d, 1H), 6.87 (d, 1H) |
| 218 | 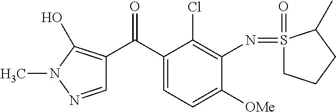 | m.p. 84-86 diastereomer mixutre 1.9:1 |

TABLE 115

| Example | Structural formula | Physical property |
|---|---|---|
| 219 | 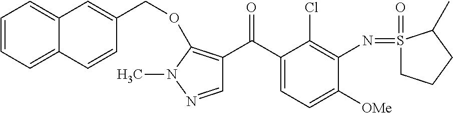 | 1H-NMR (CDCl3) 1.44 (d, 1.1/3H, l.58 (d, 1.9/3H), 1.85 (m, 2H), 2.11 (m, 4H), 3.25 (m, 4H), 3.51 (s, 3H), 3.93 (s, 3H), 5.70(s, 2H), 6.82 (d, 1H), 6.94-7.00 (m, 1H), 7.36 (s, 1H), 7.49-7.55 (m, 3H), 7.80-7.86 (m, 4H) diastereomer mixutre 1.9:1 |
| 220 | 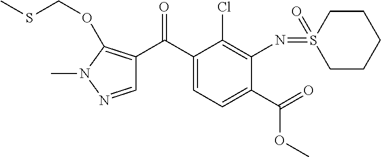 | 1H-NMR(CDCl3) 1.61 (m, 1H), 1.73 (m, 1H), 2.13 (m, 4H), 3.16 (m, 2H), 3.28 (m, 2H), 3.76 (s, 3H), 3.90 (s, 3H), 5.77 (s, 2H), 7.01 (d, 1H), 7.29 (s, 1H), 7.59 (d, 1H) |
| 221 | 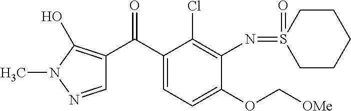 | m.p. 93-95 |
| 222 | 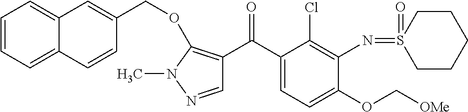 | 1H-NMR (CDCl3) 1.65 (m, 2H), 2.11 (m, 4H), 3.20 (m, 2H), 3.38 (m, 2H), 3.51 (s, 3H), 3.55 (s, 3H), 5.23 (s, 2H), 5.71 (s, 2H), 6.96 (d, 1H), 7.07 (d, 1H), 7.35 (s, 1H), 7.49-7.55 (m, 3H), 7.81- 7.86 (m, 4H) |

TABLE 116

| Example | Structural formula | Physical property |
|---|---|---|
| 223 | 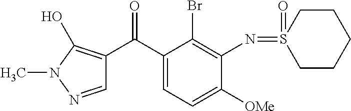 | m.p. 144-146 |
| 224 | 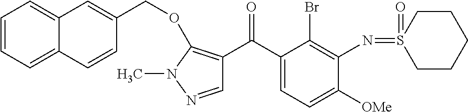 | 1H-NMR (CDCl3) 1.66 (m, 2H), 2.09 (m, 4H), 3.20 (m, 2H), 3.35 (m, 2H), 3.51 (s, 3H), 3.89 (s, 3H), 5.71 (s, 2H), 6.85 (d, 1H), 6.96 (d, 1H), 7.32 (s, 1H), 7.48- 7.55 (m, 3H), 7.81-7.86 (m, 4H) |
| 225 | 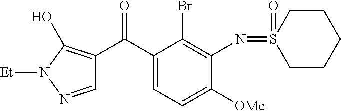 | 1H-NMR (CDCl3) 1.44 (t, 3H), 1.66 (m, 2H), 2.13 (m, 4H), 3.21 (m, 2H), 3.36 (m, 2H), 3.90 (s, 3H), 4.05 (q, 2H), 6.87 (d, 1H), 7.07 (d, 1H), 7.40 (s, 1H) |
| 226 | 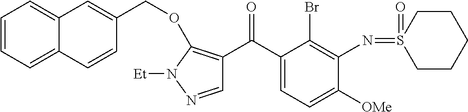 | 1H-NMR (CDCl3) 1.21 (t, 3H), 1.66 (m, 2H), 2.12 (m, 4H), 3.20 (m, 2H), 3.37 (m, 2H), 3.89 (q, 2H), 3.90 (s, 3H), 5.72 (s, 2H), 6.86 (d, 1H), 6.97 (d, 1H), 7.34 (s, 1H), 7.48-7.57 (m, 3H), 7.80-7.87 (m, 4H) |

TABLE 117

| Example | Structural formula | Physical property |
|---|---|---|
| 227 | 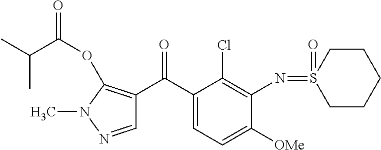 | 1H-NMR (CDCl3) 1.33 (d, 6H), 1.64 (m, 2H), 2.11 (m, 4H), 2.82 (m, 1H), 3.17 (m, 2H), 3.33 (m, 2H), 3.68 (s, 3H), 3.89 (s, 3H), 6.81 (d, 1H), 7.00 (d, 1H), 7.61 (s, 1H) |
| 228 | 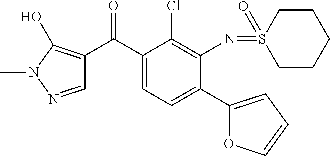 | m.p. 97-103 |
| 229 | 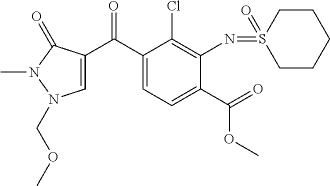 | 1H-NMR (CDCl3) 1.58 (m, 1H), 1.75 (m, 1H), 2.08 (m, 2H), 2.17 (m, 2H), 3.23 (m, 4H), 3.29 (s, 3H), 3.45 (s, 3H), 3.87 (s, 3H), 5.15 (s, 2H), 6.98 (d, 1H), 7.54 (d, 1H), 7.82 (s, 1H) |
| 230 | 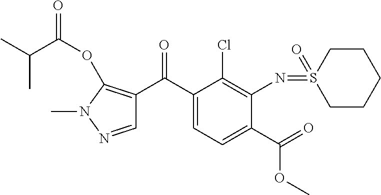 | 1H-NMR (CDC13) 1.33 (d, 6H), 1.67 (m, 2H), 2.07 (m, 2H), 2.20 (m, 2H), 3.15 (m, 2H), 3.27 (ddd, 2H), 3.68 (s, 3H), 3.89 (s, 3H), 7.00 (d, 1H), 7.55 (d, 1H), 7.59 (s, 1H) |

TABLE 118

| Example | Structural formula | Physical property |
|---|---|---|
| 231 | 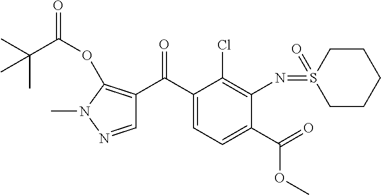 | 1H-NMR (CDCl3) 1.40 (s, 9H), 1.60(m, 1H), 1.75 (m, 1H), 2.07 (m, 2H), 2.20 (m, 2H), 3.14 (m, 2H), 3.27 (ddd, 2H), 3.68 (s, 3H), 3.89 (s, 3H), 7.02 (d, 1H), 7.48 (s, 1H), 7.57 (d, 1H) |
| 232 | 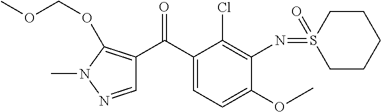 | 1H-NMR (CDCl3) 1.60 (m, 2H), 2.06 (m, 4H), 3.14 (m, 2H), 3.29 (m, 2H), 3.53 (s, 3H), 3.70 (s, 3H), 3.85 (s, 3H), 5.46 (s, 2H), 6.78 (d, 1H), 6.95 (d, 1H), 7.34 (s, 1H) |
| 233 | 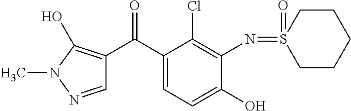 | m.p. 165-167 |
| 234 | 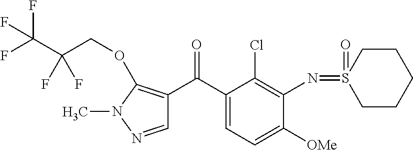 | 1H-NMR (CDCl3) 1.66 (m, 2H), 2.11 (m, 4H), 3.19 (m, 2H), 3.34 (m, 2H), 3.73 (s, 3H), 3.91 (s, 3H), 5.07 (td, 2H), 6.83 (d, 1H), 7.01 (d, 1H), 7.35 (s, 1H) |

TABLE 119

| Example | Structural formula | Physical property |
|---|---|---|
| 235 | 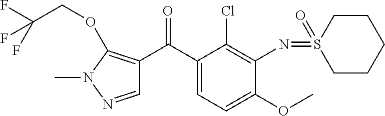 | m.p. 131-132 |
| 236 | 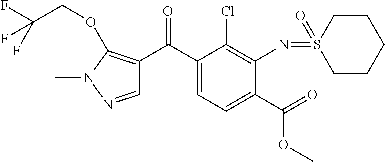 | 1H-NMR (CDCl3) 1.62 (m, 2H), 2.08 (m, 2H), 2.21 (m, 2H), 3.16 (m, 2H), 3.28 (ddd, 2H), 3.75 (s, 3H), 3.90 (s, 3H), 4.99 (q, 2H), 7.01 (d, 1H), 7.26 (s, 1H), 7.61 (d, 1H) |
| 237 | 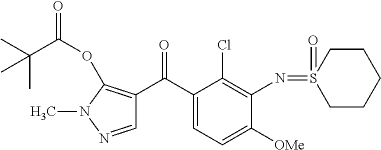 | 1H-NMR (CDCl3) 1.39 (s, 9H), 1.64 (m, 2H), 2.10 (m, 4H), 3.17 (m, 2H), 3.32 (m, 2H), 3.68 (s, 3H), 3.89 (s, 3H), 6.81 (d, 1H), 7.01 (d, 1H), 7.56 (s, 1H) |
| 238 | 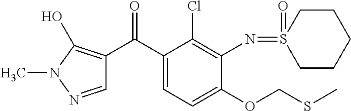 | m.p. 112-114 |

TABLE 120

| Example | Structural formula | Physical property |
|---|---|---|
| 239 | 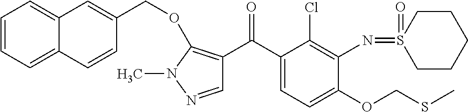 | 1H-NMR (CDCl3) 1.64 (m, 2H), 2.10 (m, 4H), 2.33 (s, 3H), 3.22 (m, 2H), 3.40 (m, 2H), 3.51 (s, 3H), 5.23 (s, 2H), 5.70 (s, 2H), 6.90 (d, 1H), 6.99 (d, 1H), 7.35 (s, 1H), 7.49-7.55 (m, 3H), 7.80-7.87 (m, 4H) |
| 240 | 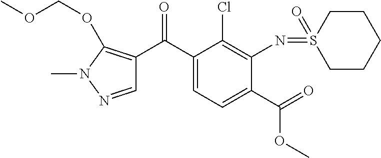 | 1H-NMR (CDCl3) 1.63 (m, 1H), 1.73 (m, 1H), 2.10 (m, 2H), 2.19 (m, 2H), 3.16 (m, 2H), 3.27 (ddd, 2H), 3.59 (s, 3H), 3.75 (s, 3H), 3.90 (s, 3H), 5.55 (s, 2H), 7.01 (d, 1H), 7.29 (s, 1H), 7.59 (d, 1H) |
| 241 | 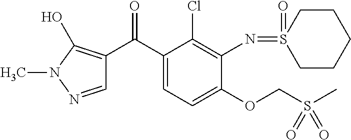 | m.p. 104-106 |
| 242 | 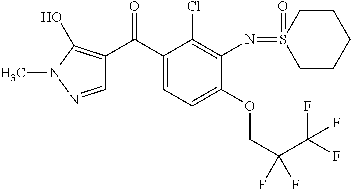 | m.p. 106-108 |

TABLE 121

| Example | Structural formula | Physical property |
|---|---|---|
| 243 | 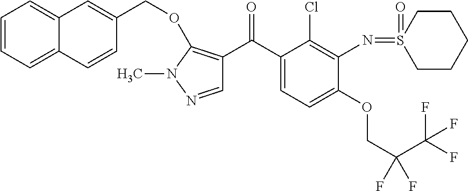 | 1H-NMR (CDCl3) 1.65 (m, 2H), 2.11 (m, 4H), 3.20 (m, 2H), 3.33 (m, 2H), 3.52 (s, 3H), 4.54 (td, 2H), 5.71 (s, 2H), 6.94 (d, 1H), 6.99 (d, 1H), 7.30 (s, 1H), 7.49-7.55 (m, 3H), 7.80-7.87 (m, 4H) |
| 244 | 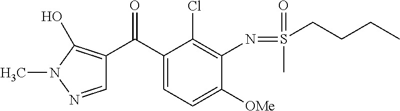 | 1H-NMR (CDCl3) 0.99 (t, 3H), 1.51 (m, 2H), 1.99 (m, 2H), 3.09 (s, 3H), 3.29 (m, 2H), 3.69 (s, 3H), 3.92 (s, 3H), 6.86 (d, 1H), 7.15 (d, 1H), 7.43 (s, 1H) |
| 245 | 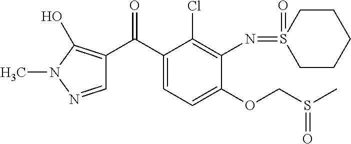 | m.p. 95-97 |
| 246 | 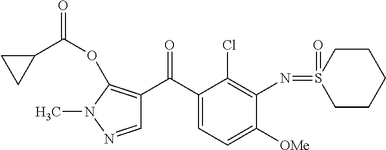 | m.p. 129-131 |

TABLE 122

| Example | Structural formula | Physical property |
|---|---|---|
| 247 | 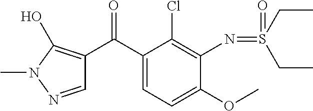 | m.p.: 165-168 |
| 248 | 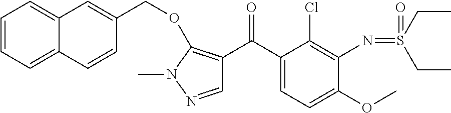 | amorphous 1H-NMR (CDCl3) 1.47 (t, 6H), 3.25 (m, 4H), 3.51 (s, 3H), 3.90 (s, 3H), 5.70 (s, 2H), 6.81 (d, 1H), 7.01 (d, 1H), 7.34 (s, 1H), 7.48- 7.55 (m, 3H), 7.80-7.86 (m, 4H) |
| 249 | 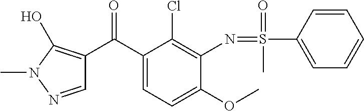 | m.p.: 88-91 |
| 250 | 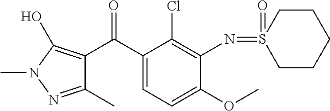 | m.p.: 138-140 |

TABLE 123

| Example | Structural formula | Physical property |
|---|---|---|
| 251 | 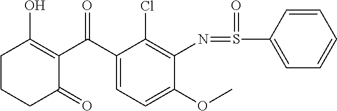 | m.p.: 152-154 |
| 252 | 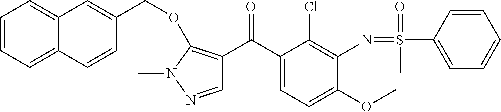 | amorphous 1H-NMR (CDCl3) 3.30 (s, 3H), 3.50 (s, 3H), 5.72 (s, 2H), 6.89 (d, 1H), 7.06 (t, 1H), 7.23 (s, 1H), 7.30 (d, 1H), 7.48-7.65 (m, 6H), 7.8l-8.06 (m, 6H) |
| 253 | 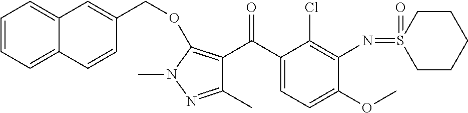 | amorphous 1H-NMR (CDCl3) 1.66 (m, 2H), 2.08 (m, 4H), 3.17 (m, 3.87 (s, 3H), 5.23 (s, 2H), 6.80 (d, 1H), 7.13 (d, 1H), 7.38-7.52 (m, 3H), 7.68-7.83 (m, 4H) |

TABLE 124

| Example | Structural formula | Physical property |
|---|---|---|
| 254 | 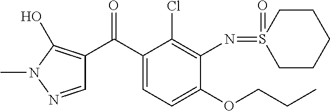 | m.p.: 85-87 |
| 255 | 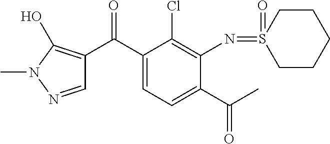 | m.p.: 100-104 |
| 256 | 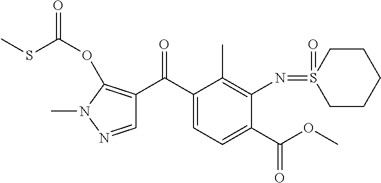 | amorphous 1H-NMR (CDCl3) 1.56 (m, 1H), 1.79 (m, 1H), 2.09 (m, 4H), 2.31 (s, 3H), 2.42 (s, 3H), 3.18 (m, 4H), 3.73 (s, 3H), 3.89 (s, 3H), 6.98 (d, 1H), 7.49 (d, 1H), 7.59 (s, 1H) |
| 257 | 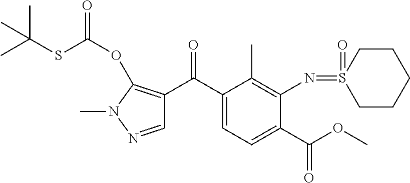 | amorphous 1H-NMR (CDCl3) 1.51 (s, 9H), 1.60 (m, 1H), 1.80 (m, 1H), 2.09 (m, 4H), 2.31 (s, 3H), 3.18 (m, 4H), 3.72 (s, 3H), 3.88 (s, 3H), 6.98 (d, 1H), 7.50 (d, 1H), 7.58 (s, 1H) |

TABLE 125

| Example | Structural formula | Physical property |
|---|---|---|
| 258 | 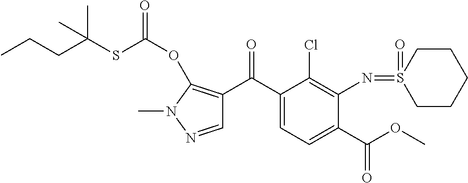 | viscous oil 1H-NMR (CDCl3) 0.97 (t, 3H), 1.38 (s, 6H), 1.40-1.50 (m, 2H), 1.62 (m, 1H), 1.71 (m, 1H), 1.68-1.80 (m, 2H), 2.09 (m, 2H), 2.20 (m, 2H), 3.14 (m, 2H), 3.26 (m, 2H), 3.68 (s, 3H), 3.89 (s, 3H), 7.02 (d, 1H), 7.44 (s, 1H), 7.57 (d, 1H) |
| 259 | 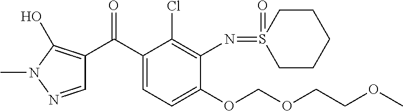 | m.p.: 77-79 |
| 260 | 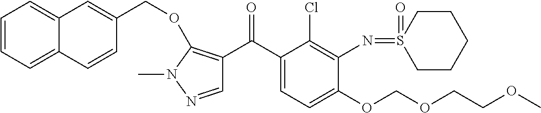 | amorphous 1H-NMR (CDCl3) 1.65 (m, 2H), 2.10 (m, 4H), 3.19 (m, 2H), 3.35 (m, 2H), 3.38 (s, 3H), 3.51 (s, 3H), 3.58 (m, 2H), 3.90 (m, 2H), 5.33 (s, 2H), 5.71 (s, 2H), 6.96 (d, 1H), 7.11 (d, 1H), 7.34 (8, 1H), 7.49- 7.55 (m, 3H), 7.81-7.87 (m, 4H) |

In addition, the compound of Example 36 was synthesized by the following method.

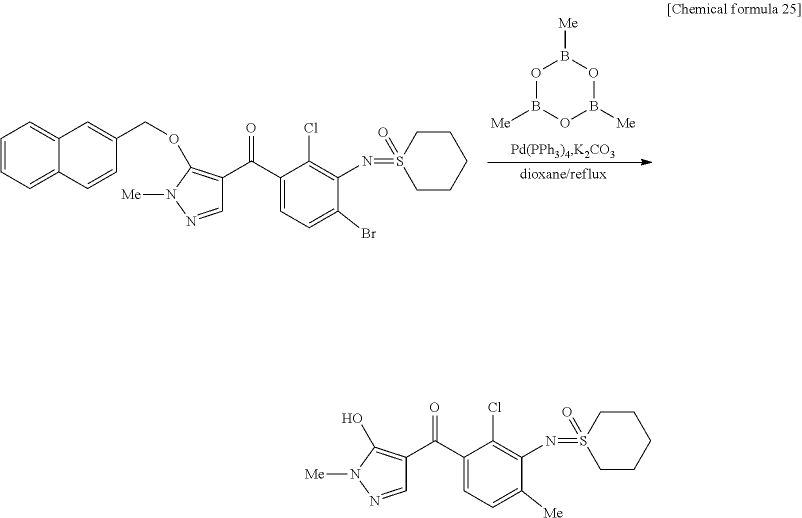

0.18 g of 4-bromo-2-chloro-1-{[1-methyl-5-(2-naphthyl methoxy)pyrazole-4-yl]carbonyl}-3-(1-oxothianylidene amino)benzene was added with 10 ml of dioxane, and replaced with nitrogen gas after deaerating under reduced pressure. The resulting solution was added with 0.17 g of potassium carbonate and 0.25 g of tetrakistriphenyl phosphine palladium under nitrogen stream, followed by performing further sufficient nitrogen gas replacement. The resulting reaction solution was added with 0.08 g of trimethyl boroxin and heated under reflux for a whole day and night. Nex, the reaction solution was cooled to room temperature, and added with 200 ml of ethyl acetate and 100 ml of water to filter insoluble matter using Celite. Celite was washed with 300 ml of ethyl acetate and 300 ml of water, and extracted the solution. The organic layer was washed with brine, dried with magnesium sulfate, filtered, concentrated. The residue was purified by silica gel column chromatography (eluent: chloroform/methanol=19/1) to obtain 0.04 g (yield: 34%) of 2-chloro-1-[(5-hydroxy-1-methylpyrazole-4-yl)carbonyl]-4-methyl-3-(1-oxothianylidene amino)benzene which is yellow-colored and amorphous.

### (3) Formulation Example

Next, some formulation examples for the herbicide of the present invention will be described. However, the active ingredients of the compounds, the additives and addition ratios are not limited to those indicated in the examples and can be varied over a wide range. In addition, the term “pails” in the formulation examples indicates parts by weight.

(Formulation example 1) Wettable Powder

| | |
|---|---|
| Compound of the present invention | 20 parts |
| White carbon | 20 parts |
| Diatomite | 52 parts |
| Sodium alkyl sulfate | 8 parts |

The above components are uniformly mixed and finely crushed to obtain a wettable powder containing 20% of the active ingredient.

(Formulation example 2) Emulsion

| | |
|---|---|
| Compound of the present invention | 20 parts |
| Xylene | 55 parts |
| Dimethyl formamide | 15 parts |
| Polyoxyethylene phenyl ether | 10 parts |

The above components are mixed and dissolved to obtain an emulsion containing 20% of the active ingredient.

(Formulation example 3) Granules

| | |
|---|---|
| Compound of the present invention | 5 parts |
| Talc | 40 parts |
| Clay | 38 parts |
| Bentonite | 10 parts |
| Sodium alkyl sulfate | 7 parts |

The above components are uniformly mixed and finely crushed to granulate particles having a diameter of 0.5 to 1.0 min and obtain granules containing 5% of the active ingredient.

### (4) Test Example

Next, Test examples demonstrating the effects of the herbicide of the present invention will be described.

### Test Example 1

Plastic pots having an area of 100 cm² and a depth of 10 cm were filled with paddy soil which is puddled by adding water, and seeds of Echinochloa crus-galli and Scirpus juncoides were planted in the pots. After seeding, rice plants at the 2.5-leaf stage were transplanted and the pots were filled with water. The rice plants were grown in a greenhouse. When Echinochloa crus-galli had grown to the 1.5-leaf stage, test solution was applied to the pots at an application dosage of 63 g per hectare. The herbicidal effects and harmful effects on the rice plants were examined after 3 weeks. The results are shown in the following table.

Herbicidal effects were examined according to the examination criteria described below and were represented by the herbicidal index.

### Examination Criteria

TABLE 11

| Herbicidal rate | Herbicidal index |
|---|---|
| 0% | 0 |
| 20-29% | 2 |
| 40-49% | 4 |
| 60-69% | 6 |
| 80-89% | 8 |
| 100% | 10 |

Numbers 1, 3, 5, 7, 9 represent values intermediate between 0 and 2, 2 and 4, 4 and 6, 6 and 8, 8 and 10.

$$Herbicidal rate ( \% ) = \frac{\begin{matrix}\{ ( Fresh weight of shoots in a non \text{-} treated plot ) \} - \\ \{ ( Fresh weight of shoots in a treated plot ) \}\end{matrix}}{( Fresh weight of shoots in a non \text{-} treated plot )} \times 100 \qquad [ Equation 1 ]$$

TABLE 12

| | Screening (63 g/ha) | | |
|---|---|---|---|
| Compound | Rice | Echinochloa crus-galli | Scripus juncoides |
| Example 1 | 0 | 10 | 8 |
| Example 2 | 0 | 8 | 8 |
| Example 3 | 0 | 8 | 8 |
| Example 4 | 0 | 10 | 10 |
| Example 5 | 0 | 10 | 10 |
| Example 6 | 0 | 10 | 10 |
| Example 7 | 0 | 5 | 8 |
| Example 8 | 0 | 10 | 9 |
| Example 9 | 0 | 10 | 7 |
| Example 10 | 0 | 7 | 7 |
| Example 11 | 2 | 10 | 8 |
| Example 12 | 0 | 10 | 9 |
| Example 13 | 0 | 7 | 8 |
| Example 14 | 1 | 7 | 8 |
| Example 15 | 2 | 10 | 8 |
| Example 16 | 3 | 10 | 10 |
| Example 17 | 0 | 7 | 8 |
| Example 18 | 0 | 7 | 8 |
| Example 20 | 0 | 0 | 4 |
| Example 21 | 0 | 10 | 9 |
| Example 22 | 0 | 0 | 5 |
| Example 23 | 0 | 7 | 8 |
| Example 24 | 0 | 7 | 8 |
| Example 25 | 0 | 10 | 9 |
| Example 26 | 0 | 7 | 8 |
| Example 27 | 0 | 8 | 7 |
| Example 29 | 0 | 10 | 8 |
| Example 30 | 0 | 10 | 8 |
| Example 31 | 0 | 10 | 8 |
| Example 33 | 0 | 7 | 6 |
| Example 34 | 0 | 6 | 5 |
| Example 36 | 0 | 10 | 9 |
| Example 37 | 2 | 7 | 8 |
| Example 38 | 3 | 8 | 8 |
| Example 39 | 0 | 9 | 8 |
| Example 40 | 0 | 7 | 8 |
| Example 41 | 0 | 4 | 6 |
| Example 42 | 0 | 3 | 6 |
| Example 43 | 0 | 2 | 5 |
| Example 44 | 0 | 6 | 7 |
| Example 45 | 0 | 10 | 5 |
| Example 48 | 0 | 7 | 8 |
| Example 50 | 0 | 5 | 8 |
| Example 51 | 0 | 5 | 8 |
| Example 52 | 0 | 10 | 9 |
| Example 53 | 0 | 10 | 9 |
| Example 54 | 0 | 10 | 8 |
| Example 55 | 0 | 6 | 8 |
| Example 56 | 0 | 7 | 9 |
| Example 57 | 0 | 10 | 7 |
| Example 58 | 0 | 6 | 8 |
| Example 59 | 2 | 6 | 8 |
| Example 61 | 3 | 8 | 8 |
| Example 63 | 0 | 7 | 8 |
| Example 65 | 0 | 10 | 5 |
| Example 69 | 1 | 10 | 9 |
| Example 70 | 0 | 7 | 8 |
| Example 72 | 0 | 10 | 5 |
| Example 74 | 2 | 10 | 9 |
| Example 75 | 0 | 6 | 6 |
| Example 76 | 0 | 7 | 7 |
| Example 80 | 0 | 9 | 8 |
| Example 82 | 0 | 5 | 8 |
| Example 83 | 0 | 7 | 8 |
| Example 84 | 0 | 6 | 8 |
| Example 85 | 0 | 6 | 6 |
| Example 86 | 0 | 8 | 8 |
| Example 87 | 0 | 9 | 8 |
| Example 88 | 0 | 8 | 8 |
| Example 89 | 0 | 6 | 7 |
| Example 90 | 0 | 9 | 8 |
| Example 93 | 0 | 7 | 8 |
| Example 95 | 0 | 10 | 8 |
| Example 96 | 0 | 7 | 7 |
| Example 97 | 0 | 7 | 7 |
| Example 99 | 0 | 9 | 8 |
| Example 100 | 0 | 7 | 6 |
| Example 102 | 0 | 6 | 6 |
| Example 104 | 0 | 10 | 9 |
| Example 105 | 0 | 10 | 8 |
| Example 106 | 0 | 10 | 8 |
| Example 107 | 0 | 7 | 7 |
| Example 108 | 0 | 10 | 8 |
| Example 109 | 0 | 7 | 8 |
| Example 110 | 0 | 10 | 8 |
| Example 111 | 0 | 6 | 8 |
| Example 112 | 0 | 8 | 8 |
| Example 113 | 0 | 7 | 6 |
| Example 114 | 0 | 10 | 8 |
| Example 115 | 0 | 10 | 8 |
| Example 116 | 0 | 7 | 8 |
| Example 117 | 0 | 5 | 8 |
| Example 118 | 0 | 7 | 8 |
| Example 119 | 0 | 10 | 8 |
| Example 120 | 0 | 8 | 5 |
| Example 121 | 0 | 7 | 7 |
| Example 122 | 0 | 10 | 6 |
| Example 123 | 0 | 7 | 5 |
| Example 126 | 0 | 10 | 8 |
| Example 127 | 0 | 8 | 8 |
| Example 128 | 0 | 10 | 8 |
| Example 129 | 0 | 7 | 8 |
| Example 130 | 0 | 10 | 8 |
| Example 131 | 0 | 6 | 8 |
| Example 132 | 0 | 6 | 8 |
| Example 133 | 0 | 6 | 7 |
| Example 134 | 0 | 5 | 8 |
| Example 135 | 0 | 5 | 7 |
| Example 136 | 0 | 7 | 8 |
| Example 137 | 0 | 10 | 8 |
| Example 138 | 0 | 10 | 8 |
| Example 139 | 0 | 10 | 8 |
| Example 145 | 0 | 10 | 8 |
| Example 146 | 0 | 5 | 6 |
| Example 148 | 0 | 6 | 8 |
| Example 149 | 0 | 5 | 6 |
| Example 150 | 0 | 5 | 8 |
| Example 151 | 0 | 6 | 5 |
| Example 152 | 0 | 10 | 8 |
| Example 153 | 0 | 7 | 8 |
| Example 154 | 0 | 10 | 8 |
| Example 155 | 0 | 8 | 8 |
| Example 158 | 0 | 5 | 5 |
| Example 161 | 0 | 9 | 8 |
| Example 165 | 0 | 5 | 8 |
| Example 166 | 0 | 6 | 8 |
| Example 173 | 0 | 5 | 7 |
| Example 177 | 0 | 5 | 7 |
| Example 179 | 1 | 7 | 7 |
| Example 181 | 0 | 10 | 9 |
| Example 182 | 0 | 10 | 7 |
| Example 183 | 0 | 7 | 6 |
| Example 185 | 0 | 10 | 7 |
| Example 187 | 0 | 10 | 5 |
| Example 188 | 1 | 10 | 7 |
| Example 191 | 0 | 6 | 7 |
| Example 193 | 0 | 6 | 7 |
| Example 196 | 0 | 6 | 8 |
| Example 197 | 0 | 7 | 7 |
| Example 198 | 0 | 5 | 7 |
| Example 201 | 0 | 5 | 7 |
| Example 202 | 0 | 7 | 7 |
| Example 203 | 0 | 10 | 7 |
| Example 204 | 0 | 5 | 7 |
| Example 207 | 0 | 6 | 7 |
| Example 208 | 0 | 8 | 7 |
| Example 209 | 0 | 7 | 6 |
| Example 210 | 0 | 6 | 9 |
| Example 211 | 0 | 6 | 8 |
| Example 218 | 0 | 6 | 7 |
| Example 247 | 0 | 10 | 9 |
| Example 248 | 0 | 5 | 8 |
| Example 250 | 0 | 7 | 8 |
| Example 253 | 0 | 6 | 8 |

### INDUSTRIAL APPLICABILITY

The composition of the present invention including as an active ingredient one or two or more types of benzoyl derivative having sulfoxyimino group, or salt thereof may be used as a herbicide which is reliably effective when used in a low dose and are highly safe.

1. A benzoyl derivative represented by formula (I) 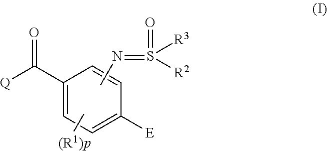
(in the formula, E represents hydrogen atom, an alkyl group, an alkenyl group, an alkynyl group, an aryl group, an alkoxy group, a cycloalkoxy group, an alkoxycarbonyl group, an alkyl thio group, cyano group, an acyl group, a heterocyclic group, NR^{a}₂ group (in the formula, each R^{a} independently represents hydrogen atom or a hydrocarbon group), R^{a}₂NC(O) group (in the formula, R^{a} is as defined above), NR^{c}C(O)R^{a} (in the formula, R^{a} is as defined above, R^{c} represents hydrogen atom or an alkyl group), NR^{c}CO₂R^{a} (in the formula, R^{a} and R^{c} are as defined above), or CR^{c}═NOR^{d} (in the formula, R^{c} is as defined above, R^{d} represents hydrogen atom or an alkyl group), when E represents NR^{a}₂ group or R^{a}₂NC(O) group, two R^{a} may bond together to form a 3- to 6-membered ring,
R¹ represents a halogen atom, hydroxyl group, mercapto group, NR^{a}₂ group (in the formula, R^{a} is as defined above), nitro group or an organic group,
p represents an integer of 0 to 3, when p is 2 or more, the numerous R¹ may be the same or different,
R² and R³ each independently represents an alkyl group, a cycloalkyl group, an alkenyl group, an alkynyl group, an aryl group or a heterocyclic group,
R² and R³ may bond together to form a 3- to 8-membered hetero ring which may include 1 to 4 nitrogen atoms, oxygen atoms or sulfur atoms other than the sulfur atom in the sulfoxyimino group,
Q represents a group selected from the groups represented by the following formulas Q1 to Q8: 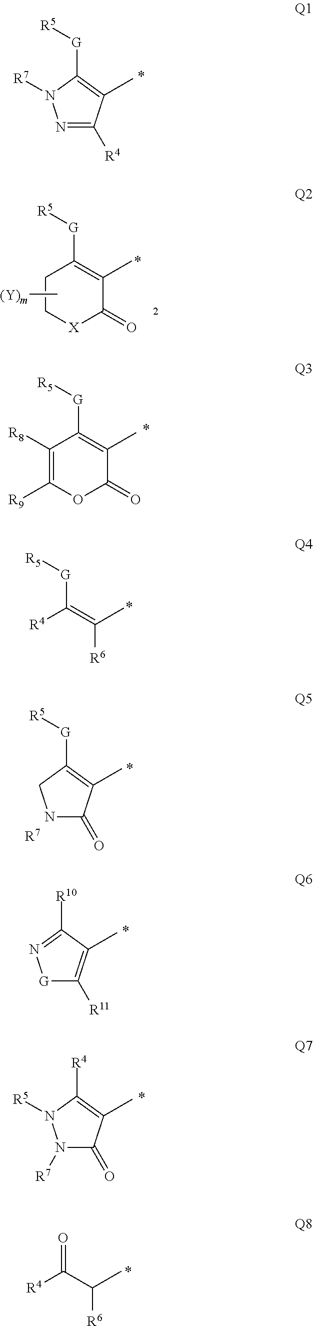
(in the formula, * represents binding site,
G represents oxygen atom, —S—, —S(O)—, —S(O)₂— or —NR^{b}— (in the formula, R^{b} represents hydrogen atom or an organic group),
R⁴ represents hydrogen atom, an alkyl group, a cycloalkyl group or NR^{a}₂ group (in the formula, R^{a} is as defined above),
R⁵ represents hydrogen atom, an alkyl group, an alkenyl group, an alkynyl group, an aryl group, a heteroaryl group, an alkoxycarbonyl group, an alkyl thiocarbonyl group, an acyl group, R^{a}₂NC(O) group (in the formula, R^{a} is as defined above), an alkylsulfonyl group, an arylsulfonyl group or NR^{a}₂SO₂ group (in the formula, R^{a} is as defined above),
R⁶ represents cyano group, an acyl group, an alkoxycarbonyl group, —C(R⁷¹)═NR⁷ group or a tetrazolyl group,
R⁷¹ represents hydrogen atom, an alkyl group, an aryl group or a heteroaryl group,
R⁷ represents hydrogen atom, an alkyl group or an alkoxy group,
R⁸ and R⁹ each independently represents hydrogen group or an alkyl group,
R¹⁰ and R¹¹ each independently represents hydrogen atom, an alkyl group or a cycloalkyl group,
X represents —C(R¹²)(R¹³)— or —N(R¹²)—,
R¹² and R¹³ each independently represents hydrogen atom or an alkyl group,
Y represents oxo group, an alkyl group, an alkoxy group, an acyl group or an alkoxycarbonyl group,
m represents an integer of 0 to 4,
when m is 2 or more, the numerous Y may be the same or different, Y may bond with each other to form a ring regardless of the substitutents listed above, Y and R¹² of X may bond together to form a ring regardless of the substitutents listed above)) or salt thereof.

2. The benzoyl derivative or salt thereof according to claim 1, wherein the benzoyl derivative is represented by formula (1-b): 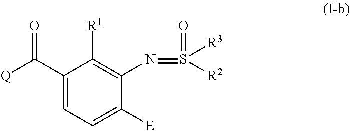
(in the formula, E, R¹ to R³, and Q are as defined above).

3. The benzoyl derivative or salt thereof according to claim 1, wherein in the formulas, R¹ represents a halogen atom, an alkyl group or —N═S(═O)R²R³ (in the formula, R² and R³ are as defined above).

4. The benzoyl derivative or salt thereof according to claim 1, wherein in the formulas, E represents an alkoxy group or an alkoxycarbonyl group.

5. The benzoyl derivative or salt thereof according to claim 3, wherein in the formulas, E represents an alkoxy group or an alkoxycarbonyl group.

6. A herbicide comprising at least one type of benzoyl derivative or salt thereof according to claim 1 as an active ingredient.

7. A herbicide comprising at least one type of benzoyl derivative or salt thereof according to claim 3 as an active ingredient.

8. A herbicide comprising at least one type of benzoyl derivative or salt thereof according to claim 4 as an active ingredient.

9. A herbicide comprising at least one type of benzoyl derivative or salt thereof according to claim 5 as an active ingredient.

10. The benzoyl derivative or salt thereof according to claim 2, wherein in the formulas, R¹ represents a halogen atom, an alkyl group or —N═S(═O)R²R³ (in the formula, R² and R³ are as defined above).

11. The benzoyl derivative or salt thereof according to claim 2, wherein in the formulas, E represents an alkoxy group or an alkoxycarbonyl group.

12. The benzoyl derivative or salt thereof according to claim 10, wherein in the formulas, E represents an alkoxy group or an alkoxycarbonyl group.

13. A herbicide comprising at least one type of benzoyl derivative or salt thereof according to claim 2 as an active ingredient.

14. A herbicide comprising at least one type of benzoyl derivative or salt thereof according to claim 10 as an active ingredient.

15. A herbicide comprising at least one type of benzoyl derivative or salt thereof according to claim 11 as an active ingredient.

16. A herbicide comprising at least one type of benzoyl derivative or salt thereof according to claim 12 as an active ingredient.
